SEQUENCE LISTING (61 sequences)

SEQ ID NO: 1 - DNA, 1638 bp, Cannabis sativa
  source 1..1638: mol_type = genomic DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgatgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 2 - DNA, 1638 bp, Cannabis sativa
  source 1..1638: mol_type = genomic DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcgtgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttmcaccgcatcatcattaa

SEQ ID NO: 3 - DNA, 1635 bp, Cannabis sativa
  source 1..1635: mol_type = genomic DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcat

SEQ ID NO: 4 - DNA, 1638 bp, Cannabis sativa
  source 1..1638: mol_type = genomic DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcgtgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 5 - DNA, 1638 bp, Cannabis sativa
  source 1..1638: mol_type = genomic DNA
atgaattgctcagcattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaa

SEQ ID NO: 6 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 7 - DNA, 20 bp, synthetic construct
  misc_feature 1..20: note = Synthetic- guide RNA
  source 1..20: mol_type = other DNA
tgcagcatggaaaatcaaac

SEQ ID NO: 8 - DNA, 20 bp, synthetic construct
  misc_feature 1..20: note = Synthetic- guide RNA
  source 1..20: mol_type = other DNA
cccttacggtggtataatgg

SEQ ID NO: 9 - DNA, 20 bp, synthetic construct
  misc_feature 1..20: note = Synthetic- guide RNA
  source 1..20: mol_type = other DNA
tagctattgaaatttggata

SEQ ID NO: 10 - DNA, 20 bp, synthetic construct
  misc_feature 1..20: note = Synthetic- guide RNA
  source 1..20: mol_type = other DNA
tagagcataaaatagttgct

SEQ ID NO: 11 - DNA, 1635 bp, Cannabis sativa
  source 1..1635: mol_type = genomic DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcgtcattaa

SEQ ID NO: 12 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 13 - DNA, 1635 bp, Cannabis sativa
  source 1..1635: mol_type = genomic DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 14 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - DNA, 1635 bp, Cannabis sativa
  misc_feature 333..441: note = n is a, c, g, or t
  source 1..1635: mol_type = genomic DNA
atgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaa

SEQ ID NO: 16 - protein, 544 aa, Cannabis sativa
  REGION 111..147: note = misc_feature - X can be any naturally occurring amino acid
MKYSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRXXXXXXXXXXXXXXXXXXXXXXXXXXXXXXXXXXXXXWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHHH

SEQ ID NO: 17 - DNA, 1634 bp, Cannabis sativa
  source 1..1634: mol_type = genomic DNA
atgaagtgctcaacattccccttttggtttgtttgcaagataatatttttctttctctcattcaatatccaaacttcaattgctaatcctcgagaaaacttccttaaatgcttctcgcaatatattcccaccaatgtaacaaatctaaaacttacacccaaaacaaccaattgtatatgcctgtccaaaattcaacaatacacaatcttagattcacctctaacacaaccccaaaactacttgttatcgtcactccttcatatgtctctcatatccaaggcactattctatgtccaagaaaattggtttgcaaattcgaactcgaagcggtggtcatgattctgaagacatgtcctacatatctcaagtcccatttgttatagtagacttgagaaacatgcattcaatcaacatagatgttcatagccaaatcgcaagggttgaagccggagctacccttggagaagtttattattgggttaatgagaaaaatgagaatcttagtttggctgctgggtattgccctactgttagcgcagctggacactttggtggaggaggatatggaccattgatgcaaaattatggcctcgcggctgataatatcgttgatgcacacttagtcaacgttgatgcaaaagtgctagatcgaaaatctatgggggaagatctcttttgggctatacgtggtggtggaggagaaagcttcggaatcattgtagcatggaaaattagactggttgctgtcccaacaaagtctactatgtttagtgttaaaaagatcatggagatacatgagcttgtcaagtgagttaacaaatggcaaaatattgcttacaagtatgacaaagatttattactcatgactcacttcataactaggaatattacaaataatcatgggaagaataagacaacaatacacacttacttctcttcagttttccttggtggagtggatagtctagtcgacttgatgaataagagttttcctgagttgggtattaaaaaaacagattgcaaacaattgagctagattgatattatcatcttttatagcggtgttgtaaattacggcactgataattttaataaggaaattttgcttgatagatcagctgggcagaacggttctttaaagattaagttagactacgttaagaaaccaattccagaatctgcgtttgtcaaaattttggaaaaattatatgaagaagatgaaggagttgggatgtatgcgttgtacccttacggtggtataatggatgagatttcagaatcagcaattccattccctcattgagctggaatcatgtatgaattatggtacatatgtagctgggagaagcacgaagataacgaaaaagcatctaaactggattcgaaatgtttatagcttcattactccttatgtgtcctaaaatccaagattggcatatctcaattatagagaccttgatactggaataaatgatcccaagagtccaaataattacacacaagaaagtatttggggtgagaagtattttggtaaaaattttgacagggtagtaaaagtgaaaaccctggttgatcccaataatttttttagaaatgaacaaagcatcccacctcttccacggcatcgtcattaa

SEQ ID NO: 18 - DNA, 20 bp, synthetic construct
  misc_feature 1..20: note = Synthetic- guide RNA
  source 1..20: mol_type = other DNA
gctagatcgaaaatctatgg

SEQ ID NO: 19 - DNA, 20 bp, synthetic construct
  misc_feature 1..20: note = Synthetic- guide RNA
  source 1..20: mol_type = other DNA
aaagcatcccacctcttcca

SEQ ID NO: 20 - DNA, 20 bp, synthetic construct
  misc_feature 1..20: note = Synthetic- guide RNA
  source 1..20: mol_type = other DNA
tttaggacagacatatacaa

SEQ ID NO: 21 - DNA, 20 bp, synthetic construct
  misc_feature 1..20: note = Synthetic- guide RNA
  source 1..20: mol_type = other DNA
gaaagcaccgttctgcccag

SEQ ID NO: 22 - DNA, 20 bp, synthetic construct
  misc_feature 1..20: note = Synthetic- guide RNA
  source 1..20: mol_type = other DNA
catttaaggaagttttctcg

SEQ ID NO: 23 - DNA, 20 bp, synthetic construct
  misc_feature 1..20: note = Synthetic- guide RNA
  source 1..20: mol_type = other DNA
aaatgggacttgagatatgt

SEQ ID NO: 24 - DNA, 20 bp, synthetic construct
  misc_feature 1..20: note = Synthetic- guide RNA
  source 1..20: mol_type = other DNA
cccttggagaagtttattat

SEQ ID NO: 25 - DNA, 20 bp, synthetic construct
  misc_feature 1..20: note = Synthetic- guide RNA
  source 1..20: mol_type = other DNA
gtacccttacggtggtataa

SEQ ID NO: 26 - DNA, 20 bp, synthetic construct
  misc_feature 1..20: note = Synthetic- guide RNA
  source 1..20: mol_type = other DNA
attccagctcgatgagggaa

SEQ ID NO: 27 - DNA, 20 bp, synthetic construct
  misc_feature 1..20: note = Synthetic- guide RNA
  source 1..20: mol_type = other DNA
tacacacaagcacgtatttg

SEQ ID NO: 28 - protein, 109 aa, Brassica napus
MATGSRVLIGLAMILIISGELLVPGQGTCQGDIEGLMRECAVYVQRPGPKVNPSAACCKVVKRSDIPCACGRITPSVQKMIDMNKVVLVTSFCGRPLAHGTKCGSYIVP

SEQ ID NO: 29 - DNA, 330 bp, Brassica napus
  source 1..330: mol_type = genomic DNA
atggcgacaggttctcgtgttctgatcggtctagcaatgatcctcataatctcaggagaactgctagttccagggcaaggaacgtgccaaggagacatagagggtctgatgagagaatgtgcggtctacgtccagcgtccaggcccaaaggtaaacccatccgcagcgtgttgcaaagtcgtcaagagatcagacatcccctgcgcatgtggccgtatcacaccctcggttcaaaaaatgatagacatgaataaggttgttcttgtcacttccttttgtgggaggcctctcgctcatggtaccaagtgtggaagctacattgtgccatga

SEQ ID NO: 30 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAVLCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 31 - DNA, 1188 bp, Cannabis sativa
  source 1..1188: mol_type = genomic DNA
atgggactctcatcagtttgtaccttttcatttcaaactaattaccatactttattaaatcctcacaataataatcccaaaacctcattattatgttatcgacaccccaaaacaccaattaaatactcttacaataattttccctctaaacattgctccaccaagagttttcatctacaaaacaaatgctcagaatcattatcaatcgcaaaaaattccattagggcagctactacaaatcaaactgagcctccagaatctgataatcattcagtagcaactaaaattttaaactttgggaaggcatgttggaaacttcaaagaccatatacaatcatagcatttacttcatgcgcttgtggattgtttgggaaagagttgttgcataacacaaatttaataagttggtctctgatgttcaaggcattcttttttttggtggctgtattatgcattgcttcttttacaactaccatcaatcagatttacgatcttcacattgacagaataaacaagcctgatctaccactagcttcaggggaaatatcagtaaacacagcttggattatgagcataattgtggcactgtttggattgataataactataaaaatgaagggtggaccactctatatatttggctactgttttggtatttttggtgggattgtctattctgttccaccatttagatggaagcaaaatccttccactgcatttcttctcaatttcctggcccatattattacaaatttcacattttattatgccagcagagcagctcttggcctaccatttgagttgaggccttcttttactttcctgctagcatttatgaaatcaatgggttcagctttggctttaatcaaagatgcttcagacgttgaaggcgacactaaatttggcatatcaaccttggcaagtaaatatggttccagaaacttgacattattttgttctggaattgttctcctatcctatgtggctgctatacttgctgggattatctggccccaggctttcaacagtaacgtaatgttactttctcatgcaatcttagcattttggttaatcctccagactcgagattttgcgttaacaaattacgacccggaagcaggcagaagattttacgagttcatgtggaagctttattatgctgaatatttagtatatgttttcatataa

SEQ ID NO: 32 - protein, 370 aa, Cannabis sativa
MGSTGIETQMTPTQISDEEANLFAMQLASASVLPMVLKAALELDLLEIIAKAGPGAFLSPSDIAQQLPTQNPDAPVMLDRMLRLLASYNVVTYSLRERETAEEEGKVERLYGLAPVSKYLTKNEDGVSIAPLCLMNQDKVLMESWYHLKDAVLDGGIPFNKAYGMTAFEYHGTDQRFNKIFNRGMSDHSTITMKKILETYKGFEGLNSIVDVGGGTGAVVNMIVSKYPTIKGINFDLPHVIEDAPPLTGVEHVGGDMFVSVPKGDAIFMKWICHDWSDEHCLKFLKNCHAALPEHGKVIVAECILPVAPDSSLATKSTVHIDVIMLAHNPGGKERTEKEFEALAKGAGFKGFKVHCNAFNTHIMEFLKTI

SEQ ID NO: 33 - DNA, 1113 bp, Cannabis sativa
  source 1..1113: mol_type = genomic DNA
atgggttcaacaggaatagagacccaaatgaccccaacccaaatatccgacgaagaagccaacctcttcgccatgcaattagccagtgcctcagtcttacccatggttctcaaagcagctttagagctcgacctcttggagatcatagccaaggccggtccaggcgcgtttctctcaccttccgacatagctcaacagcttccgactcagaacccagacgccccggtgatgctggaccggatgctgagactgttggctagctacaacgtggtgacgtactcgctgcgtgagcgtgagacggcggaagaggaagggaaggtggagaggctttatgggttggctccggtgagtaaatatctgacgaagaatgaagatggagtctccattgctcctctttgtctcatgaaccaggataaggttcttatggagagttggtatcacttaaaagatgcagtacttgatggaggaatacctttcaacaaggcatatggaatgacagcatttgaatatcatggaaccgatcaaaggttcaataaaatctttaatagaggaatgtccgaccactcgactattaccatgaaaaaaatcctcgaaacttacaagggtttcgagggtcttaactcgattgttgatgttggtggtggtactggagctgttgttaacatgatcgtctctaagtaccctactattaagggtattaacttcgatttgcctcatgtcatcgaagatgcacctccattgaccggtgtagagcatgttggaggagacatgtttgtaagtgtaccaaaaggagatgcaattttcatgaagtggatttgccatgattggagcgatgaacactgcttgaaattcttgaagaactgccacgctgcactgcccgaacacggaaaagtgatcgtggcggagtgcattcttccggtggcaccggactcgagccttgccacaaagagtacggtccacattgatgtgatcatgttggcccataaccctggtggcaaagagagaacagagaaagagtttgaggcattggctaagggagctggctttaaaggcttcaaagtccattgcaatgctttcaatacccatatcatggaatttctcaagaccatttaa

SEQ ID NO: 34 - protein, 400 aa, Cannabis sativa
MVFSSVCSFPSSLGTNFKLVPRSNFKASSSHYHEINNFINNKPIKFSYFSSRLYCSAKPIVHRENKFTKSFSLSHLQRKSSIKAHGEIEADGSNGTSEFNVMKSGNAIWRFVRPYAAKGVLFNSAAMFAKELVGNLNLFSWPLMFKILSFTLVILCIFVSTSGINQIYDLDIDRLNKPNLPVASGEISVELAWLLTIVCTISGLTLTIITNSGPFFPFLYSASIFFGFLYSAPPFRWKKNPFTACFCNVMLYVGTSVGVYYACKASLGLPANWSPAFCLLFWFISLLSIPISIAKDLSDIEGDRKFGIITFSTKFGAKPIAYICHGLMLLNYVSVMAAAIIWPQFFNSSVILLSHAFMAIWVLYQAWILEKSNYATETCQKYYIFLWIIFSLEHAFYLFM

SEQ ID NO: 35 - DNA, 1203 bp, Cannabis sativa
  source 1..1203: mol_type = genomic DNA
atggtgttctcatcagtttgtagttttccatcctcccttggaactaattttaaattagttcctcgtagtaattttaaggcatcatcttctcattatcatgaaataaataattttattaataataaaccaattaaattctcatatttttcttcaagactatattgctctgccaaaccaattgtacacagagaaaacaaattcacaaaatcattttcactcagccacctccaaaggaaaagctccataaaggcacatggtgaaattgaagctgatgggagtaatggcacatctgaatttaatgtaatgaaaagtggaaacgcaatttggagatttgtaaggccatatgcagccaagggagtattgtttaactctgctgctatgtttgcaaaagagttggtggggaacctaaatctatttagttggcctttgatgtttaagatactctcttttacattggttattttatgcatttttgtaagtacaagtggcatcaatcaaatttatgatctcgacatcgacaggttaaacaaacctaatttgccagtagcatcaggagaaatttcagttgaattggcatggttgttgactatagtttgtacaataagtggcctcacattaacaattataacgaactcagggccattcttcccttttctctactctgctagtatcttttttggctttctctattctgctcctccattcagatggaagaagaatccttttacagcatgtttctgtaatgttatgttgtatgttggcacaagcgttggtgtctattatgcttgtaaggctagtctcgggcttccagccaactggagccctgctttttgtttgctcttttggtttatttcattgttgagtatacccatctccattgcaaaagatctttcagacatagaaggtgaccgcaagtttggaatcataaccttctcaactaaatttggagcaaaacccatagcatatatttgtcatggactcatgcttctgaattacgtgagtgttatggctgcagctattatttggccacagtttttcaacagtagcgtaatattgctttctcatgcattcatggcaatttgggtattatatcaggcttggatattggagaaatcaaattacgccacggagacgtgccaaaaatactatatattcctttggataattttttctcttgaacatgccttctatttgttcatgtag

SEQ ID NO: 36 - protein, 523 aa, Cannabis sativa
MAQVSKICSNGAQTILTLPNISKSHTPRSLNSVSLRSPFLGSSNSLSLKIGTEFGGCSTVGKAMAGPVMASAVTAEKPSKVPEIVLQPIKDISGTVKLPGSKSLSNRILLLAALSEGTTVVDNLLDSDDIHYMLGALETLGLRVEADKESKRAIVEGCAGQFPAGKESVDEVQLFLGNAGTAMRPLTAAVTVAGGNASYVLDGVPRMRERPIGDLVTGLKQLGADVDCFHGTDCPPVRVLGKGGLPGGKVKLSGSISSQYLTALLMAAPLALGDVEIEIIDKLISVPYVDMTLKLMARFGVTVEHSDSWDRFLVKGGQKYKSPGNAYVEGDASSASYFLAGAAVTGGTVTVEGCGTSSLQGDVKFAEVLEKMGAKVSWTENSVTVTGPPRDSVKSKHLKAIDVNMNKMPDVAMTLAVVALFADGPTAIRDVASWRVKETERMIAICTELRKLGATVEEGPDYCVITPPEKLNITAIDTYDDHRMAMAFSLAACSDVPVTIKDPGCTRKTFPDYFEVLERFTKH

SEQ ID NO: 37 - protein, 523 aa, synthetic construct
  REGION 1..523: note = Synthetic- engineered protein
MAQVSKICSNGAQTILTLPNISKSHTPRSLNSVSLRSPFLGSSNSLSLKIGTEFGGCSTVGKAMAGPVMASAVTAEKPSKVPEIVLQPIKDISGTVKLPGSKSLSNRILLLAALSEGTTVVDNLLDSDDIHYMLGALETLGLRVEADKESKRAIVEGCAGQFPAGKESVDEVQLFLGNAGIAMRSLTAAVTVAGGNASYVLDGVPRMRERPIGDLVTGLKQLGADVDCFHGTDCPPVRVLGKGGLPGGKVKLSGSISSQYLTALLMAAPLALGDVEIEIIDKLISVPYVDMTLKLMARFGVTVEHSDSWDRFLVKGGQKYKSPGNAYVEGDASSASYFLAGAAVTGGTVTVEGCGTSSLQGDVKFAEVLEKMGAKVSWTENSVTVTGPPRDSVKSKHLKAIDVNMNKMPDVAMTLAVVALFADGPTAIRDVASWRVKETERMIAICTELRKLGATVEEGLDYCVITPPEKLNITAIDTYDDHRMAMAFSLAACSDVPVTIKDPGCTRKTFPDYFEVLERFTKH

SEQ ID NO: 38 - DNA, 1572 bp, Cannabis sativa
  source 1..1572: mol_type = genomic DNA
atggcccaagtgagcaaaatctgtagcaatggagctcaaactatccttactctcccaaatatatctaagtctcatacaccaagatccctaaattcagtttcgttgagatcaccgtttttgggttcatctaactctttgagtttgaagattggaactgaatttgggggttgttctacggttggtaaagctatggctggtccagtcatggcttcagctgtcacagcggagaagccttcaaaggtaccggagattgtgttgcagcccattaaagatatctctggcactgtcaagttgccgggttccaagtcactatcgaatcggattctactcctggctgctctttctgaggggacaactgttgtggacaacttgttagatagtgatgacattcactacatgcttggtgccttggaaacccttggtcttcgtgttgaagcagacaaggaaagcaaacgagcaattgtggaaggttgtgcgggtcagtttcctgcaggtaaagaatctgttgacgaagttcaacttttccttggaaatgctggaacagcaatgcgtccactcacagctgcggtgactgttgctggtggaaatgctagctacgtacttgatggtgttcctcgaatgagagaaagaccaattggagatttggtgactggtcttaagcagcttggtgcagatgttgattgttttcatggtacggattgtccccctgttcgtgtgcttggaaaaggaggccttcctgggggcaaggtgaaactttctggatcaattagcagtcaatatttgacagccttgcttatggcagctcccttggctcttggagatgttgaaatcgagataattgataaattgatctcggttccctatgttgatatgactttgaagttgatggcacgttttggggttactgttgaacacagtgatagctgggatcgatttttagttaaaggaggtcaaaagtacaaatctcctggaaacgcttatgttgaaggtgatgcttcaagtgctagttacttcctagctggtgctgcagtcactggtggtacagtcaccgtagaaggttgtgggactagtagtttacagggagacgtaaaatttgctgaagttcttgagaaaatgggtgctaaagttagctggacagagaacagtgtcacggtcactggaccaccacgagattctgtaaaaagtaaacacttgaaagccattgatgtcaacatgaacaaaatgcctgatgttgccatgactcttgctgtagttgctctttttgctgatggccccactgctataagagatgtggcaagttggagagtcaaggagacagagagaatgattgccatctgcactgaactcagaaagcttggagcaacagttgaggaaggacccgattactgcgtgatcactccaccagagaaactaaatatcacagcaatagacacatacgacgaccacaggatggctatggcgttctctcttgcagcttgttcagatgtgccagttaccattaaggatcctggttgcacccgaaaaactttcccagattactttgaagtccttgagagatttacaaagcactga

SEQ ID NO: 39 - DNA, 1572 bp, synthetic construct
  misc_feature 1..1572: note = Synthetic- engineered gene
  source 1..1572: mol_type = other DNA
atggcccaagtgagcaaaatctgtagcaatggagctcaaactatccttactctcccaaatatatctaagtctcatacaccaagatccctaaattcagtttcgttgagatcaccgtttttgggttcatctaactctttgagtttgaagattggaactgaatttgggggttgttctacggttggtaaagctatggctggtccagtcatggcttcagctgtcacagcggagaagccttcaaaggtaccggagattgtgttgcagcccattaaagatatctctggcactgtcaagttgccgggttccaagtcactatcgaatcggattctactcctggctgctctttctgaggggacaactgttgtggacaacttgttagatagtgatgacattcactacatgcttggtgccttggaaacccttggtcttcgtgttgaagcagacaaggaaagcaaacgagcaattgtggaaggttgtgcgggtcagtttcctgcaggtaaagaatctgttgacgaagttcaacttttccttggaaatgctggaatagcaatgcgttcactcacagctgcggtgactgttgctggtggaaatgctagctacgtacttgatggtgttcctcgaatgagagaaagaccaattggagatttggtgactggtcttaagcagcttggtgcagatgttgattgttttcatggtacggattgtccccctgttcgtgtgcttggaaaaggaggccttcctgggggcaaggtgaaactttctggatcaattagcagtcaatatttgacagccttgcttatggcagctcccttggctcttggagatgttgaaatcgagataattgataaattgatctcggttccctatgttgatatgactttgaagttgatggcacgttttggggttactgttgaacacagtgatagctgggatcgatttttagttaaaggaggtcaaaagtacaaatctcctggaaacgcttatgttgaaggtgatgcttcaagtgctagttacttcctagctggtgctgcagtcactggtggtacagtcaccgtagaaggttgtgggactagtagtttacagggagacgtaaaatttgctgaagttcttgagaaaatgggtgctaaagttagctggacagagaacagtgtcacggtcactggaccaccacgagattctgtaaaaagtaaacacttgaaagccattgatgtcaacatgaacaaaatgcctgatgttgccatgactcttgctgtagttgctctttttgctgatggccccactgctataagagatgtggcaagttggagagtcaaggagacagagagaatgattgccatctgcactgaactcagaaagcttggagcaacagttgaggaaggacttgattactgcgtgatcactccaccagagaaactaaatatcacagcaatagacacatacgacgaccacaggatggctatggcgttctctcttgcagcttgttcagatgtgccagttaccattaaggatcctggttgcacccgaaaaactttcccagattactttgaagtccttgagagatttacaaagcactga

SEQ ID NO: 40 - DNA, 4196 bp, Cannabis sativa
  source 1..4196: mol_type = genomic DNA
ggttggtaagccctcctaccctctttgaaaattgaaagagagtcaatgtcgacctacagcagcagcatccattaacgttaccattgccaccaaaaatccaacctttatttgtatagagagaatcagagaaggtttgggtttcagagagagagagaagaagaacaaaaaaatggcccaagtgagcaaaatctgtagcaatggagctcaaactatccttactctcccaaatatatctaagtctcatacaccaagatccctaaattcagtttcgttgagatcaccgtttttgggttcatctaactctttgagtttgaagattggaactgaatttgggggttgttctacggttggtaaagctatggctggtccagtcatggcttcagctgtcacagcggagaagccttcaaaggtaccggagattgtgttgcagcccattaaagatatctctggcactgtcaagttgccgggttccaagtcactatcgaatcggattctactcctggctgctctttctgaggtatatttcattttttttaaaacgtcaaacatgtatttttgtcgaggaagttttctgtatatacaaagataagagagtaaaaatatggaacatcaataccaaaatgaaccaaaactaggctaagctatcaaatcatgtcatggtatgccatactctactttcctatctcaagctccacagctataaaatactatatcgtaattattttgtcaactgctttcatattccttgtaatttccctcattcccactaaaactagttccaatggattgtgtggctggaaactgtagttagttacattagctagatctgaaccatgatcagcatcgactgcccaactggtaaaccatgtaattgcatggaattcttcctttgttatccacaaatttgaaaagtatttttgaggtatacaaagattgtgctttttatgagcaattttcttttagttttatgttaagagtttgtagcgatgggatgttttttttctagaaaatggacagtaaagcttagcatttttactttattggtgtaaatgaatagtgttcattgaagctgaactcatgcccttaattgggaggaaaattgagagaaatggagtaaagtaatatgatattttggttaaattcgtaagaatatgatggaaataaaaaatgcaactcaactgggttactgaagttatatttctggtctcagttgtgcttttacaactttagtctagagctccacgctgcggagagattcggagtccttacagtttattttgataatgatttatgagaatttcataactctacgcttttgttacattatatatgaggtgtttcgttggtgcattgtctcacctgaactccctaaattttagaatgtgggatttagaatgaagttatactattagtgtttgagtcatctagaatttgtagctgctcattctccatatactctttctctatttcctccccatattttggcgctactacttatctttacagttcatgttattttcatgtacttgagttttttgccctataaaatattttgagcggtgggaagtaactgttttttttgttataattatccaggggacaactgttgtggacaacttgttagatagtgatgacattcactacatgcttggtgccttggaaacccttggtcttcgtgttgaagcagacaaggaaagcaaacgagcaattgtggaaggttgtgcgggtcagtttcctgcaggtaaagaatctgttgacgaagttcaacttttccttggaaatgctggaacagcaatgcgtccactcacagctgcggtgactgttgctggtggaaatgctaggtttgtcttcattgcaattgcttttgaatataaagtacttctaatgcagtgaatttatgctcttgtttttcttactggccgagtagctcttacattttaggtaaagaaagtcacttttgctaacaacatcaccatttatacttccctctttactttgatgtggttatgctagaaattacatgttggaaatgaactagcacatatcataaattattttgtatgctgttattacattttctcagtaacctcttaacttctatatctcagctacgtacttgatggtgttcctcgaatgagagaaagaccaattggagatttggtgactggtcttaagcagcttggtgcagatgttgattgttttcatggtacggattgtccccctgttcgtgtgcttggaaaaggaggccttcctgggggcaaggtgaggcttgcattgcttcttcttattctttttggccataaaacatcattgtaatagtggttttatgttatgaaatccattgactggtttatttttaggttgttgttttgcttttaaataaaaacaatattgtcaaatgatgcataagtagtgattacatctacatcatttaatttattatcttaaatgatgacaaacttcatcattttgactcagaattatgtaatattaccctttgcaggtgaaactttctggatcaattagcagtcaatatttgacagccttgcttatggcagctcccttggctcttggagatgttgaaatcgagataattgataaattgatctcggttccctatgttgatatgactttgaagttgatggcacgttttggggttactgttgaacacagtgatagctgggatcgatttttagttaaaggaggtcaaaagtacaagtaggtttcttctgaatatagttgatagtattgttacattacatctggttatgtcaaagagtaataaattgaaaaataaaaatctgtcagatctcctggaaacgcttatgttgaaggtgatgcttcaagtgctagttacttcctagctggtgctgcagtcactggtggtacagtcaccgtagaaggttgtgggactagtagtttacaggtattttgcttagaccttgaaatctcttattcttgtacttgtgtttacatagaatctaagattaagtgtatttacatacattaactggtgtttaataaagggagacgtaaaatttgctgaagttcttgagaaaatgggtgctaaagttagctggacagagaacagtgtcacggtcactggaccaccacgagattctgtaaaaagtaaacacttgaaagccattgatgtcaacatgaacaaaatgcctgatgttgccatgactcttgctgtagttgctctttttgctgatggccccactgctataagagatggtatgtttttccttaaatttgtgagatggtaaaatggggcagtcggtttgggttggggtagattatcggttctcgtctgccataataaaaaataatctgctcatttgcaataaatttcacagacacaaaaatgaaaaccaataaaatattattttgtttagaggattaaatactcatttcttgcctttcctaattcccagtggcaagttggagagtcaaggagacagagagaatgattgccatctgcactgaactcagaaaggttagtttttatgctgttttatgtacttgttatgtcatgcgccttggaatgtaatggctgatagctatctgttcttatgggaacaaacatttcagcttggagcaacagttgaggaaggacccgattactgcgtgatcactccaccagagaaactaaatatcacagcaatagacacatacgacgaccacaggatggctatggcgttctctcttgcagcttgttcagatgtgccagttaccattaaggatcctggttgcacccgaaaaactttcccagattactttgaagtccttgagagatttacaaagcactgaatgagtatttattaactggatagagaacaatagcatcggctactgtcattacaactaaagcagttgggaggcaggcaatccttttcaattatcatgtgtttgattttggtcgactgtattgcaagttgagcttctcattattattaagactgtaatcgtagttatttgttgtaacttctgcaaacccttcatgttattttttcacccttctaataagccagtggggcaaattctatatctgctatatgagcttgagtgtagagagaacttttgtcaatgtataggtttctagcagaagcaccatccctaatatgctttattataagagttgctgtgatcgtgtagtgttattttattgaaagtaaccgacgcatctcatatta

SEQ ID NO: 41 - DNA, 46 bp, synthetic construct
  misc_feature 1..46: note = Synthetic- prime editing guide RNA
  source 1..46: mol_type = other DNA
tgaagactttgcacaacttttccttggaaatgcgtttaagtcttct

SEQ ID NO: 42 - DNA, 46 bp, synthetic construct
  misc_feature 1..46: note = Synthetic- prime editing guide RNA
  source 1..46: mol_type = other DNA
agaagacttaaacgcatttccaaggaaaagttgtgcaaagtcttca

SEQ ID NO: 43 - DNA, 64 bp, synthetic construct
  misc_feature 1..64: note = Synthetic- engineered gene
  source 1..64: mol_type = other DNA
acctgctatagtgctgagtgaacgcattgctattccagcatttccaaggaaacatatagcaggt

SEQ ID NO: 44 - DNA, 46 bp, synthetic construct
  misc_feature 1..46: note = Synthetic- prime editing guide RNA
  source 1..46: mol_type = other DNA
tgaagactttgcacttggagcaacagttgaggagtttaagtcttct

SEQ ID NO: 45 - DNA, 46 bp, synthetic construct
  misc_feature 1..46: note = Synthetic- prime editing guide RNA
  source 1..46: mol_type = other DNA
agaagacttaaactcctcaactgttgctccaagtgcaaagtcttca

SEQ ID NO: 46 - DNA, 60 bp, synthetic construct
  misc_feature 1..60: note = Synthetic- engineered gene
  source 1..60: mol_type = other DNA
acctgctatagtgccacgcagtaatcaagtccttcctcaactgttgaacatatagcaggt

SEQ ID NO: 47 - DNA, 349 bp, synthetic construct
  misc_feature 1..349: note = Synthetic- engineered gene
  source 1..349: mol_type = other DNA
ggtctcaaatggcgacaggttctcgtgttctgatcggtctagcaatgatcctcataatctcaggagaactgctagttccagggcaaggaacgtgccaaggagacatagagggtctgatgagagaatgtgcggtctacgtccagcgtccaggcccaaaggtaaacccatccgcagcgtgttgcaaagtcgtcaagagatcagacatcccctgcgcatgtggccgtatcacaccctcggttcaaaaaatgatagacatgaataaggttgttcttgtcacttccttttgtgggaggcctctcgctcatggtaccaagtgtggaagctacattgtgccatgagcttagagacc

SEQ ID NO: 48 - DNA, 348 bp, synthetic construct
  misc_feature 1..348: note = Synthetic- engineered gene
  source 1..348: mol_type = other DNA
ggtctcaaatggcgacaggttctcgtgttctgatcggtctagcaatgatcctcataatctcaggagaactgctagttccagggcaaggaacgtgccaaggagacatagagggtctgatgagagaatgtgcggtctacgtccagcgtccaggcccaaaggtaaacccatccgcagcgtgttgcaaagtcgtcaagagatcagacatcccctgcgcatgtggccgtatcacaccctcggttcaaaaaatgatagacatgaataaggttgttcttgtcacttccttttgtgggaggcctctcgctcatggtaccaagtgtggaagctacattgtgccaagttcgagagacc

SEQ ID NO: 49 - DNA, 1207 bp, synthetic construct
  misc_feature 1..1207: note = Synthetic- engineered gene
  source 1..1207: mol_type = other DNA
ggtctcaaatgggactctcatcagtttgtaccttttcatttcaaactaattaccatactttattaaatcctcacaataataatcccaaaacctcattattatgttatcgacaccccaaaacaccaattaaatactcttacaataattttccctctaaacattgctccaccaagagttttcatctacaaaacaaatgctcagaatcattatcaatcgcaaaaaattccattagggcagctactacaaatcaaactgagcctccagaatctgataatcattcagtagcaactaaaattttaaactttgggaaggcatgttggaaacttcaaagaccatatacaatcatagcatttacttcatgcgcttgtggattgtttgggaaagagttgttgcataacacaaatttaataagttggtcactgatgttcaaggcattcttttttttggtggctgtattatgcattgcttcttttacaactaccatcaatcagatttacgatcttcacattgacagaataaacaagcctgatctaccactagcttcaggggaaatatcagtaaacacagcttggattatgagcataattgtggcactgtttggattgataataactataaaaatgaagggtggaccactctatatatttggctactgttttggtatttttggtgggattgtctattctgttccaccatttagatggaagcaaaatccttccactgcatttcttctcaatttcctggcccatattattacaaatttcacattttattatgccagcagagcagctcttggcctaccatttgagttgaggccttcttttactttcctgctagcatttatgaaatcaatgggttcagctttggctttaatcaaagatgcttcagacgttgaaggcgacactaaatttggcatatcaaccttggcaagtaaatatggttccagaaacttgacattattttgttctggaattgttctcctatcctatgtggctgctatacttgctgggattatctggccccaggctttcaacagtaacgtaatgttactttctcatgcaatcttagcattttggttaatcctccagactcgagattttgcgttaacaaattacgacccggaagcaggcagaagattttacgagttcatgtggaagctttattatgctgaatatttagtatatgttttcatataagcttagagacc

SEQ ID NO: 50 - DNA, 1206 bp, synthetic construct
  misc_feature 1..1206: note = Synthetic- engineered gene
  source 1..1206: mol_type = other DNA
ggtctcaaatgggactctcatcagtttgtaccttttcatttcaaactaattaccatactttattaaatcctcacaataataatcccaaaacctcattattatgttatcgacaccccaaaacaccaattaaatactcttacaataattttccctctaaacattgctccaccaagagttttcatctacaaaacaaatgctcagaatcattatcaatcgcaaaaaattccattagggcagctactacaaatcaaactgagcctccagaatctgataatcattcagtagcaactaaaattttaaactttgggaaggcatgttggaaacttcaaagaccatatacaatcatagcatttacttcatgcgcttgtggattgtttgggaaagagttgttgcataacacaaatttaataagttggtcactgatgttcaaggcattcttttttttggtggctgtattatgcattgcttcttttacaactaccatcaatcagatttacgatcttcacattgacagaataaacaagcctgatctaccactagcttcaggggaaatatcagtaaacacagcttggattatgagcataattgtggcactgtttggattgataataactataaaaatgaagggtggaccactctatatatttggctactgttttggtatttttggtgggattgtctattctgttccaccatttagatggaagcaaaatccttccactgcatttcttctcaatttcctggcccatattattacaaatttcacattttattatgccagcagagcagctcttggcctaccatttgagttgaggccttcttttactttcctgctagcatttatgaaatcaatgggttcagctttggctttaatcaaagatgcttcagacgttgaaggcgacactaaatttggcatatcaaccttggcaagtaaatatggttccagaaacttgacattattttgttctggaattgttctcctatcctatgtggctgctatacttgctgggattatctggccccaggctttcaacagtaacgtaatgttactttctcatgcaatcttagcattttggttaatcctccagactcgagattttgcgttaacaaattacgacccggaagcaggcagaagattttacgagttcatgtggaagctttattatgctgaatatttagtatatgttttcataagttcgagagacc

SEQ ID NO: 51 - DNA, 1132 bp, synthetic construct
  misc_feature 1..1132: note = Synthetic- engineered gene
  source 1..1132: mol_type = other DNA
ggtctcaaatgggttcaacaggaatagaaacccaaatgaccccaacccaaatatccgacgaagaagccaacctcttcgccatgcaattagccagtgcctcagtcttacccatggttctcaaagcagctttagagctcgacctcttggagatcatagccaaggccggtccaggcgcgtttctctcaccttccgacatagctcaacagcttccgactcagaacccagacgccccggtgatgctggaccggatgctgagactgttggctagctacaacgtggtgacgtactcgctgcgtgagcgtgaaacggcggaagaggaagggaaggtggagaggctttatgggttggctccggtgagtaaatatctgacgaagaatgaagatggagtctccattgctcctctttgtctcatgaaccaggataaggttcttatggagagttggtatcacttaaaagatgcagtacttgatggaggaatacctttcaacaaggcatatggaatgacagcatttgaatatcatggaaccgatcaaaggttcaataaaatctttaatagaggaatgtccgaccactcgactattaccatgaaaaaaatcctcgaaacttacaagggtttcgagggtcttaactcgattgttgatgttggtggtggtactggagctgttgttaacatgatcgtttctaagtaccctactattaagggtattaacttcgatttgcctcatgtcatcgaagatgcacctccattgaccggtgtagagcatgttggaggagacatgtttgtaagtgtaccaaaaggagatgcaattttcatgaagtggatttgccatgattggagcgatgaacactgcttgaaattcttgaagaactgccacgctgcactgcccgaacacggaaaagtgatcgtggcggagtgcattcttccggtggcaccggactcgagccttgccacaaagagtacggtccacattgatgtgatcatgttggcccataaccctggtggcaaagagagaacagagaaagagtttgaggcattggctaagggagctggctttaaaggcttcaaagtccattgcaatgctttcaatacccatatcatggaatttctcaagaccatttaagcttagagacc

SEQ ID NO: 52 - DNA, 1131 bp, synthetic construct
  misc_feature 1..1131: note = Synthetic- engineered gene
  source 1..1131: mol_type = other DNA
ggtctcaaatgggttcaacaggaatagaaacccaaatgaccccaacccaaatatccgacgaagaagccaacctcttcgccatgcaattagccagtgcctcagtcttacccatggttctcaaagcagctttagagctcgacctcttggagatcatagccaaggccggtccaggcgcgtttctctcaccttccgacatagctcaacagcttccgactcagaacccagacgccccggtgatgctggaccggatgctgagactgttggctagctacaacgtggtgacgtactcgctgcgtgagcgtgaaacggcggaagaggaagggaaggtggagaggctttatgggttggctccggtgagtaaatatctgacgaagaatgaagatggagtctccattgctcctctttgtctcatgaaccaggataaggttcttatggagagttggtatcacttaaaagatgcagtacttgatggaggaatacctttcaacaaggcatatggaatgacagcatttgaatatcatggaaccgatcaaaggttcaataaaatctttaatagaggaatgtccgaccactcgactattaccatgaaaaaaatcctcgaaacttacaagggtttcgagggtcttaactcgattgttgatgttggtggtggtactggagctgttgttaacatgatcgtttctaagtaccctactattaagggtattaacttcgatttgcctcatgtcatcgaagatgcacctccattgaccggtgtagagcatgttggaggagacatgtttgtaagtgtaccaaaaggagatgcaattttcatgaagtggatttgccatgattggagcgatgaacactgcttgaaattcttgaagaactgccacgctgcactgcccgaacacggaaaagtgatcgtggcggagtgcattcttccggtggcaccggactcgagccttgccacaaagagtacggtccacattgatgtgatcatgttggcccataaccctggtggcaaagagagaacagagaaagagtttgaggcattggctaagggagctggctttaaaggcttcaaagtccattgcaatgctttcaatacccatatcatggaatttctcaagaccattagttcgagagacc

SEQ ID NO: 53 - DNA, 1222 bp, synthetic construct
  misc_feature 1..1222: note = Synthetic- engineered gene
  source 1..1222: mol_type = other DNA
ggtctcaaatggtgttctcatcagtttgtagttttccatcctcccttggaactaattttaaattagttcctcgtagtaattttaaggcatcatcttctcattatcatgaaataaataattttattaataataaaccaattaaattctcatatttttcttcaagactatattgctctgccaaaccaattgtacacagagaaaacaaattcacaaaatcattttcactcagccacctccaaaggaaaagctccataaaggcacatggtgaaattgaagctgatgggagtaatggcacatctgaatttaatgtaatgaaaagtggaaacgcaatttggagatttgtaaggccatatgcagccaagggagtattgtttaactctgctgctatgtttgcaaaagagttggtggggaacctaaatctatttagttggcctttgatgtttaagatactctcttttacattggttattttatgcatttttgtaagtacaagtggcatcaatcaaatttatgatctcgacatcgacaggttaaacaaacctaatttgccagtagcatcaggagaaatttcagttgaattggcatggttgttgactatagtttgtacaataagtggcctcacattaacaattataacgaactcagggccattcttcccttttctctactctgctagtatcttttttggctttctctattctgctcctccattcagatggaagaagaatccttttacagcatgtttctgtaatgttatgttgtatgttggcacaagcgttggtgtctattatgcttgtaaggctagtctcgggcttccagccaactggagccctgctttttgtttgctcttttggtttatttcattgttgagtatacccatctccattgcaaaagatctttcagacatagaaggtgaccgcaagtttggaatcataaccttctcaactaaatttggagcaaaacccatagcatatatttgtcatggactcatgcttctgaattacgtgagtgttatggctgcagctattatttggccacagtttttcaacagtagcgtaatattgctttctcatgcattcatggcaatttgggtattatatcaggcttggatattggagaaatcaaattacgccacggaaacgtgccaaaaatactatatattcctttggataattttttctcttgaacatgccttctatttgttcatgtaggcttagagacc

SEQ ID NO: 54 - DNA, 1221 bp, synthetic construct
  misc_feature 1..1221: note = Synthetic- engineered gene
  source 1..1221: mol_type = other DNA
ggtctcaaatggtgttctcatcagtttgtagttttccatcctcccttggaactaattttaaattagttcctcgtagtaattttaaggcatcatcttctcattatcatgaaataaataattttattaataataaaccaattaaattctcatatttttcttcaagactatattgctctgccaaaccaattgtacacagagaaaacaaattcacaaaatcattttcactcagccacctccaaaggaaaagctccataaaggcacatggtgaaattgaagctgatgggagtaatggcacatctgaatttaatgtaatgaaaagtggaaacgcaatttggagatttgtaaggccatatgcagccaagggagtattgtttaactctgctgctatgtttgcaaaagagttggtggggaacctaaatctatttagttggcctttgatgtttaagatactctcttttacattggttattttatgcatttttgtaagtacaagtggcatcaatcaaatttatgatctcgacatcgacaggttaaacaaacctaatttgccagtagcatcaggagaaatttcagttgaattggcatggttgttgactatagtttgtacaataagtggcctcacattaacaattataacgaactcagggccattcttcccttttctctactctgctagtatcttttttggctttctctattctgctcctccattcagatggaagaagaatccttttacagcatgtttctgtaatgttatgttgtatgttggcacaagcgttggtgtctattatgcttgtaaggctagtctcgggcttccagccaactggagccctgctttttgtttgctcttttggtttatttcattgttgagtatacccatctccattgcaaaagatctttcagacatagaaggtgaccgcaagtttggaatcataaccttctcaactaaatttggagcaaaacccatagcatatatttgtcatggactcatgcttctgaattacgtgagtgttatggctgcagctattatttggccacagtttttcaacagtagcgtaatattgctttctcatgcattcatggcaatttgggtattatatcaggcttggatattggagaaatcaaattacgccacggaaacgtgccaaaaatactatatattcctttggataattttttctcttgaacatgccttctatttgttcatgagttcgagagacc

SEQ ID NO: 55 - DNA, 61 bp, Cannabis sativa
  source 1..61: mol_type = genomic DNA
ttgacgaagttcaacttttccttggaaatgctggaacagcaatgcgtccactcacagctgc

SEQ ID NO: 56 - DNA, 57 bp, synthetic construct
  misc_feature 1..57: note = Synthetic- engineered gene
  misc_feature 21: note = N represents any nucleotide
  source 1..57: mol_type = other DNA
caacttttccttggaaatgcntgagtgaacgcattgctattccagcatttccaagga

SEQ ID NO: 57 - DNA, 56 bp, Cannabis sativa
  source 1..56: mol_type = genomic DNA
cttggagcaacagttgaggaaggacccgattactgcgtgatcactccaccagagaa

SEQ ID NO: 58 - DNA, 53 bp, synthetic construct
  misc_feature 1..53: note = Synthetic- engineered gene
  misc_feature 21: note = N represents any nucleotide
  source 1..53: mol_type = other DNA
cttggagcaacagttgaggancacgcagtaatcaagtccttcctcaactgttg

SEQ ID NO: 59 - DNA, 1638 bp, synthetic construct
  misc_feature 1..1638: note = In silico- consensus sequence
  source 1..1638: mol_type = other DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 60 - DNA, 1737 bp, Arabidopsis thaliana
  source 1..1737: mol_type = genomic DNA
atgaaggagatcgcgatgaggaattcaaagcgcaagcctgagccgacgccgttcgccgggaagaagctccggtcgacgcgattacgccggaagagagcacagatctctcccgttcttgttcaatcacctctctggagcaaacaaatcggagtctctgctgcttctgtcgattcctgctccgatttgctagctgatgacaacgtttcctgtggttcgagcagagtcgagaagagctcgaatccgaagaagactctaattgaagaggtagaagtttctaaacctggttataatgtgaaggagacgattggtgattcgaaatttcgaaggattacgaggtcttactctaagctacacaaggagaaggagggagatgagatcgaagtaagcgaatcgtcttgtgttgattcgaattctggtgctggattaaggagattgaatgtgaagggaaataaaattaacgacaacgatgagatctctttctcacgatccgatgtgaccttcgccggacatgtctccaacagccggagtttgaatttcgaatcggagaataaggagagcgacgtcgtttctgtcatatctggagttgagtactgttccaagttcgggagcgttaccggaggagctgataacgaagaaattgaaatctccaagccgagcagcttcgtggaagctgattcctctcttggatcggccaaggaattgaagccggagcttgagatagtcggatgcgtctctgatctcgcttgctctgagaaattctcggaagaggtttcggattctctcgatgatgagtcatctgagcaacgttcagagatatattcacagtattccgacttcgattactcggattacactccgtccatcttcttcgactctggcagcgaattctctgagaaatcttcctctgattctcctatttcacattctcgctctctgtacctccagttcaaggaacagttctgtagatccacgattcccaacgattttggatcttcttgcgaggaagaaattcactctgaattgctaaggtttgatgatgaggaggtggaagagagctatctaaggctgagggaaagagaaagaagtcatgcatatatgcgggactgtgctaaggcatactgctccaggatggacaatactggtctcatccctcgtctacgctccatcatggttcaatggattgtaaagcaatgttctgacatggggcttcagcaagagacattgtttctaggagttggtctgttggatcgattcctgagcaaaggatcattcaaaagcgaaaggactctaatactagtcgggattgcgagtcttactctggccaccagaattgaagaaaatcaaccttacaacagcatccggaaaaggaacttcaccattcagaacctaagatatagccggcatgaagtggtggcaatggagtggctggttcaagaagtcctcaacttcaaatgcttcacacccacaatcttcaacttcttgtggttctacttaaaagctgctcgagccaatccagaagttgaaaggaaagccaaatccttggctgttacctcactatccgaccaaactcaactctgtttttggccctcaactgtagcagctgcactcgtggttctcgcctgcatcgaacacaacaaaatctctgcataccaacgagtcataaaggtccatgttagaacaacagataacgagttgcctgaatgcgttaagagtctggactggttgcttgggcagtaa

SEQ ID NO: 61 - DNA, 832 bp, Bacillus amyloliquefaciens
  source 1..832: mol_type = genomic DNA
ctggaaaacgtcacattgcttccgcatatcgggtcagcaacggctaaaatccgcttgaatatgttcacacaagccgctcaaaacatgattgacgccgtatacggaagaacgccgaaaaaccttactaaggaatttcaataagaagaaaaatcccggttggttcagccggggtttatttttcgctagataaaaagtactatttttaaattctttctattcctttctttcgttgctgatacaatgaaaaggaatcagcttcacatgatgaaaatgggaggtattgctttgaaaaaacgattatcgtggatttccgtttgtttactggtgcttgtctccgcggcggggatgctgttttcaacagctgccaaaacggaaacatcttctcacaaggcacacacagaagcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataacgaaaaaaacggcttccctgcggaggccgtttttttcagctttacataaagtgtgtaataaatttttcttcaaactctgatcggtcaatttcacttt

(19) United States
(12) Patent Application Publication
(10) Pub. No.: US 2025/0101444 A1
(43) Pub. Date: Mar. 27, 2025

(54) METHODS OF GENE EDITING AND TRANSFORMING CANNABIS

(71) Applicant: Wisconsin Alumni Research Foundation, Madison, WI (US)

(72) Inventors: Michael W. Petersen, Merrimac, WI (US); Edward James Williams, Madison, WI (US); Robert Harnish, Middleton, WI (US); Heidi Flewelling Kaeppler, Oregon, WI (US); Brian Martinell, Mt. Horeb, WI (US); Ray Collier, Middleton, WI (US); Frank McFarland, Madison, WI (US); Shawn Michael Kaeppler, Oregon, WI (US)

(21) Appl. No.: 18/649,801

(22) Filed: Apr. 29, 2024

Related U.S. Application Data
(62) Division of application No. 16/930,936, filed on Jul. 16, 2020, now Pat. No. 11,999,958.
(60) Provisional application No. 62/982,522, filed on Feb. 27, 2020.
(60) Provisional application No. 62/906,210, filed on Sep. 26, 2019.
(60) Provisional application No. 62/875,311, filed on Jul. 17, 2019.

(51) Int. Cl. C12N 15/82; A01H 4/00; A01H 6/28; C07K 14/415

(52) U.S. Cl. CPC C12N 15/8201; A01H 4/005; A01H 6/28; C07K 14/415

(57) ABSTRACT

Disclosed herein are methods for the production of Cannabis meristem explants from dry seeds. Also described are methods of transforming and gene editing using the Cannabis meristem explants disclosed herein.

### CROSS-REFERENCE TO RELATED APPLICATIONS

This application is a divisional of U.S. application Ser. No. 16/930,936, filed Jul. 16, 2020, which application claims priority to U.S. Provisional Application No. 62/875,311, filed Jul. 17, 2019, U.S. Provisional Application No. 62/906,210, filed Sep. 26, 2019, and U.S. Provisional Application No. 62/982,522, filed Feb. 27, 2020, each of which is incorporated herein by reference in their entirety.

### STATEMENT REGARDING FEDERALLY SPONSORED RESEARCH

Not Applicable.

### SEQUENCE LISTING

A Sequence Listing accompanies this application and is submitted in XML format titled “2024-10-25_960296-04523_WIPO_Sequence_Listing_XML.xml” which is 111,079 bytes in size and was created on Oct. 25, 2024. The sequence listing is electronically submitted via Patent Center and is incorporated herein by reference in its entirety.

### BACKGROUND

A key hurdle in the transformation of plants is the responsiveness of cells in tissue culture to produce embryogenic cells that go on to form clonal, intact and fertile plants. Many species and varieties of plants are recalcitrant to this type of regeneration, including Cannabis sativa and Cannabis indica. Therefore, a need in the art exists for improved Cannabis transformation and gene editing methods.

### SUMMARY OF THE INVENTION

In a first aspect, provided herein is a method of preparing an explant, the method comprising the steps of, rehydrating a dry Cannabis seed in a hydration medium, and excising meristematic tissue from the rehydrated Cannabis seed to form an explant. In some embodiments, the hydration medium comprises one or more priming agents. In some embodiments, the seed is a Cannabis sativa seed.

In some embodiments, the explant preparation method additionally comprises the step of surface sterilizing the Cannabis seed prior to rehydration. In some embodiments, the seed is surface sterilized using bleach.

In some embodiments, the explant preparation method additionally comprises the step of drying the explant. In some embodiments, the dried explant is capable of being stored for at least 10 days. In some embodiments, the explant is dried in the presence of one or more transformation supplements. In some embodiments, the transformation supplement is selected from the group consisting of a small molecule, a nucleic acid, a polypeptide, a protein, an antibody, a transcription factor, a biological macromolecule, a nanoparticle, and a liposome.

In some embodiments, the explant preparation method additionally comprise the step of transforming the explant with a heterologous nucleic acid of interest. In some embodiments, the explant is transformed using Agrobacterium mediated transformation or particle bombardment.

In a second aspect, provided herein is a Cannabis explant generated by the explant preparation methods described herein.

In a third aspect, provided herein is a dried Cannabis explant generated by the methods described herein.

In a forth aspect, provided herein is a method of transforming Cannabis with a heterologous nucleic acid, the method comprising the steps of rehydrating a dry Cannabis seed in a hydration medium, excising meristematic tissue from the rehydrated Cannabis seed to form a Cannabis explant, incubating the Cannabis explant in a pretreatment medium, inoculating the Cannabis explant with Agrobacterium spp. comprising the heterologous nucleic acid, co-culturing the Cannabis explant in a co-culture medium for between about 1 day and about 6 days, and culturing the Cannabis explant on a selection medium to select for transformed Cannabis explants.

In some embodiments, the method of transforming Cannabis additionally comprises the step of force treating the Cannabis explant prior to or following inoculation. In some embodiments, the force treatment is selected from the group consisting of sonication, vortexing, centrifugation, heat-shock, and addition of chemicals.

In some embodiments, the Cannabis transformation method additionally comprises the step of surface sterilizing the Cannabis seed prior to rehydration.

In some embodiments, the heterologous nucleic acid modulates the expression or activity of an endogenous Cannabis gene selected from the group consisting of tetrahydrocannabinolic acid synthase (THCA synthase), cannabidiolic acid synthase (CBDA synthase), O-methyltransferase (CsOMT21), lipid transfer protein 2 (LTP2), prenyltransferase 3 (CsPT3), and prenyltransferase 1 (CsPT1). In some embodiments, the heterologous nucleic acid encodes a polypeptide at least 90% identical to SEQ ID NO:28. In some embodiments, the heterologous nucleic acid encodes a guide RNA that targets Cannabis sativa THCA synthase gene, Cannabis sativa CBDA synthase gene, or Cannabis sativa EPSP synthase gene

In some embodiments, the Cannabis seed is a Cannabis sativa seed. In some embodiments, the Cannabis seed is a seed from a Cannabis plant with less than 0.3 percent THC based on dry weight.

In a fifth aspect, provided herein is a transformed Cannabis explant produced by the methods described herein.

In a sixth aspect, provided herein is a Cannabis plant grown from the Cannabis explant generated by the methods described herein.

In a seventh aspect, provided herein is a method of producing a transformed Cannabis seed, the method comprising contacting a female Cannabis flower with an Agrobacterium spp. culture, wherein the Agrobacterium comprises a heterologous nucleic acid and pollinating the contacted female Cannabis flower with male pollen from a suitable donor plant, whereby a transformed Cannabis seed is produced. In some embodiments, the Agrobacterium spp. culture comprises sucrose and a wetting agent. In some embodiments, the Agrobacterium comprises a vector comprising the heterologous nucleic acid.

In an eighth aspect, provided herein is a method of transforming a Cannabis plant, the method comprising growing a sanitized and imbibed Cannabis seed on a non-selective culture medium suitable for supporting the growth and survival of the Cannabis seed until a Cannabis explant is formed, inoculating the Cannabis explant with a heterologous nucleic acid, co-culturing the Cannabis explant in a co-culture medium for between about 1 day and about 6 days, and culturing the Cannabis explant on a selection medium to select for transformed Cannabis explants. In some embodiments, the explant is selected from the group consisting of a leaf explant, a node explant, an internode explant, a petiole explant, a hypocotyl explant, and a bud explant. In some embodiments, the Cannabis explant is inoculated using particle bombardment, high velocity microprojection, microinjection, electroporation, direct DNA uptake, cell-penetrating peptides, silica carbide fibers, nanoparticles, and bacterially-mediated transformation. In some embodiments, Agrobacterium spp. is used to inoculate the Cannabis explant.

In some embodiments, the method additionally comprises the step of force treating the Cannabis explant prior to or following inoculation. In some embodiments, the force treatment is selected from the group consisting of sonication, vortexing, centrifugation, heat-shock, and addition of chemicals.

In some embodiments, the heterologous nucleic acid modulates the expression or activity of an endogenous Cannabis gene selected from the group consisting of tetrahydrocannabinolic acid synthase (THCA synthase), cannabidiolic acid synthase (CBDA synthase), O-methyltransferase (CsOMT21), lipid transfer protein 2 (LTP2), prenyltransferase 3 (CsPT3), and prenyltransferase 1 (CsPT1). In some embodiments, the heterologous nucleic acid encodes a polypeptide at least 90% identical to SEQ ID NO:28. In some embodiments, the heterologous nucleic acid encodes a guide RNA that targets Cannabis sativa THCA synthase gene, Cannabis sativa CBDA synthase gene, or Cannabis sativa EPSP synthase gene.

In some embodiments, the Cannabis seed is a Cannabis sativa seed.

### BRIEF DESCRIPTION OF DRAWINGS

The patent or patent application file contains at least one drawing in color. Copies of this patent or patent application publication with color drawings will be provided by the Office upon request and payment of the necessary fee.

FIG. 1 shows stable RFP expression in Cannabis plantlet from meristem transformation experiments outlined in Example 1.

FIG. 2 shows a non-transgenic Cannabis plant derived from meristem explant in tissue culture.

FIG. 3 shows transient GUS expression in Cannabis meristem explants varieties Honey Gold 3WS and Fiber Hemp inoculated with Ar18r12v/DICOTBINARY-19.

FIG. 4 shows the plasmid map of the DICOTBINARY-22 vector.

FIG. 5 shows flower phenotypes post inoculum application (1 day).

FIG. 6 shows GUS transient expression in Cannabis leaves, petioles, internodes, and nodes inoculated with Ar18r12v/DICOTBINARY-19.

FIG. 7 shows mature Cannabis seed and embryo.

FIG. 8 shows Cannabis seeds and meristem explants.

FIG. 9 shows Cannabis meristem explants on B5 medium and after 4 days in co-culture after inoculation with Ar18r12v/DICOTBINARY-19.

FIG. 10 shows transient GUS expression in Cannabis meristem explants transformed with Ar18r12v/DICOTBINARY-19.

FIG. 11 shows transient GUS expression in Cannabis meristem explants transformed with Ar18r12v/DICOTBINARY-19 and de-stained in 70% EtOH.

FIG. 12 shows a non-inoculated Cannabis seed sanitized in 20% Clorox on B5 medium (left) and a non-inoculated meristem explant on B5 medium (right).

FIG. 13 shows spectinomycin sensitive (bleaching) phenotype visible in inoculated Cannabis meristem explants on 150 mg/L spectinomycin B5.

FIG. 14 shows sanitized Cannabis seeds and axenic plantlets on B5 medium after about 6 weeks.

FIG. 15 shows greening, regenerating Cannabis explants that were dried, stored, and regenerated (right) against freshly excised explants (left) that were plated on Hemp Node medium (Table 7) the day of excision. Explants were imaged approximately 3 weeks after excision.

FIG. 16 shows stable GUS expression in a plantlet derived from the treatment Hemp 3/22-3B described in Example 1. One of the leaves in a plantlet transferred to non-selective BRM expressed GUS in leaf stably.

FIG. 17 shows Cannabis seeds imbibed at 37° C.

FIG. 18 shows embryonic parts produced by mechanical excision.

FIG. 19 shows the plasmid map of control binary construct SOYTEST-2.

FIG. 20 shows positive GUS transients in Cannabis meristem explants (3WS variety) using SOYTEST-2 in both the Ar18r12v (left) and GV3101 (right) strains of Agrobacterium, demonstrating transfection of Cannabis meristems using disarmed strains of both Agrobacterium rhizogenes (Ar18r12v) and Agrobacterium tumefaciens (GV3101).

FIG. 21 additional stable RFP expressing Cannabis (Plant WP421-1) from experiments in the Honey Gold 3WS variety. The plantlet in the center is rooting on 50 mg/L streptomycin hemp node media (after being on 50 mg/L spectinomycin hemp node media for approximately 1 month).

FIG. 22 shows the plasmid map of the DICOTBOMB-13 vector.

FIG. 23 shows GUS transient expression in Cannabis meristem explants bombarded with DICOTBOMB-13.

FIG. 24 shows pollen germination results.

FIG. 25 shows pollen germination results. T0=initial germination after 1 hr treatment; T1=germination after 4 hr treatment; T2 =germination after overnight treatment (22 h).

FIG. 26 shows the phenotype of plant WP421-1 on the day of transfer from tissue culture to the greenhouse (right) and after approximately 4 weeks in the greenhouse (left).

FIG. 27 shows stable RFP and GUS expression in leaves of TO Cannabis plant WP421-1 derived from meristem transformation.

FIG. 28 shows stable GUS expression of leaves of TO Cannabis plant WP421-1 derived from meristem transformation.

FIG. 29 shows Cannabis VAEs after 5 weeks (left) compared to freshly excised Cannabis meristem explants (right).

FIG. 30 shows machine excised Cannabis meristem explants imaged after approximately two weeks on non-selective B5 medium.

FIG. 31 shows transgenic TO plant WP421-1 after 7 weeks in the greenhouse.

FIG. 32 shows GUS expression in leaf samples from WP421-1. Nine out of the ten leaf samples showed positive GUS expression confirming minimal chimerism.

FIG. 33 shows the PCR amplification scheme for PCR of leaf samples from WP421-1. A 156 bp fragment within the aadA1a expression cassette of DICOTBINARY-19 was amplified using primers designated F56 and R11. The fragments and the resulting amplicon is highlighted in blue.

FIG. 34 shows the results of PCR amplification of a 156 bp fragment of the aadA1a expression cassette. All 10 leaf samples were positive.

FIG. 35 shows bleached Cannabis meristem explants after 1 month on 100 mg/L spectinomycin node medium. RFP negative Cannabis meristem explants that were previously greening on 10-50 mg/L spectinomycin were transferred to hemp node medium containing 100 mg/L spectinomycin.

FIG. 36 shows GUS expression in vascular tissue (petiole sections) of Cannabis T0 event WP421-1 (“Candice”).

FIG. 37 shows pollination of WP421-1 by 3WS wild-type plant.

FIG. 38 shows flower of WP421-1 pollinated by 3WS wild-type plant.

FIG. 39 shows cuttings of WP421-1.

FIG. 40 shows T1 seedlings of WP421-1 and RFP expression in T1 seedling WP421-1@2 “Carly.”

FIG. 41 shows germline confirmation of Cannabis meristem transformation through RFP, GUS expression, and aadA1a PCR.

FIG. 42 shows T1 Cannabis plants WP421-1@1 and WP421-1@2 after 2 months in greenhouse.

FIG. 43 shows T1 seed of WP421-1.

FIG. 44 shows stable RFP expression (tdTomato) in T1 seedlings of WP421-1 (germinated on germination paper).

FIG. 45 shows stable RFP expression (tdTomato) in T1 seedlings of WP421-1 (germinated in flats).

FIG. 46 shows stable RFP expression (tdTomato) in T1 seedlings of WP421-1 (germinated in flats).

FIG. 47 shows stable GUS expression in T1 seedlings of WP421-1 (germinated in flats).

FIG. 48 shows GUS expression 7-8 weeks post-inoculation in chimeric Cannabis shoots (3WS variety) regenerated on 75 mg/L spectinomycin.

FIG. 49 shows T0 Cannabis plant WP421-2 (Honey Gold 3WS +DICOTBINARY-19).

FIG. 50 shows Cannabis meristem explants targeted on carboxymethylcellulose media for particle bombardment.

FIG. 51 shows phenotype and stable GUS expression in transgenic 3WS Cannabis derived from particle-mediated transformation of meristem explants (imaged approximately 2 months post-blast).

FIG. 52 shows phenotype and stable GUS expression in TO transgenic 3WS Cannabis plant WP-001181-1a (“Fernanda”) derived from particle-mediated transformation of meristem explants.

FIG. 53 shows phenotype of T0 transgenic 3WS Cannabis plant WP-001181-1a “Fernanda” derived from particle-mediated transformation of meristem explants after approximately 3 weeks in greenhouse.

FIG. 54 shows Cannabis WP001181-1a particle gun T0 event aadA1a PCR.

FIG. 55 shows Cannabis WP001181-1a particle gun T0 event GUS expression.

FIG. 56 shows GUS expression in vascular tissue in petiole sections (and corresponding leaf) of Cannabis T0 particle gun event WP001181-1a.

FIG. 57 shows phenotype and stable GUS expression in T0 transgenic 3WS Cannabis plant WP-001181-1b (“Hernanda”) derived from particle-mediated transformation of meristem explants.

FIG. 58 shows GUS transient expression in automated-excised Cannabis meristem explants of Fiber Hemp variety post co-culture.

FIG. 59 shows metal rolling pin with glass plate; optional gap can be set by adding/removing shims (metal shims shown on left, paper on right).

FIG. 60 shows transient GUS expression in Cannabis (Abacus variety) meristem explants mechanically excised by crushing under a rolling pin, then stored for 2 months at −20 C (left image seed crushed wet and crushed material then dried; right image seed dried first then crushed.

FIG. 61 shows GUS transient expression in Cannabis meristem explants of variety 3WS and Abacus post co-culture.

FIG. 62 shows RFP (tdTomato) segregation in the T2 generation of transformed Cannabis (derived from T0 event WP421-1).

FIG. 63 shows a schematic of the gene networks underlying cannabinoid and terpenoid accumulation in Cannabis. Zager et al., Plant Physiology, 2019, 180 (4): 1877-1897.

FIG. 64 shows a sequence alignment of various THCA synthase cDNA sequences. From top to bottom, the sequences shown are: SEQ ID NO:1, SEQ ID NO:3, SEQ ID NO:4, SEQ ID NO:2, SEQ ID NO:5, and SEQ ID NO:59.

FIG. 65 shows a sequence alignment of various CBDA synthase cDNA sequences. From top to bottom, the sequences shown are: SEQ ID NO:11, SEQ ID NO:13, SEQ ID NO:15, SEQ ID NO:11, SEQ ID NO:17, and SEQ ID NO:11.

FIG. 66 shows embodiments of a cloning strategy for BraLTP2. From top to bottom, the sequences shown are: SEQ ID NO:29, SEQ ID NO:47, and SEQ ID NO:48.

FIG. 67 shows embodiments of a cloning strategy for CsPT1. From top to bottom, the sequences shown are: SEQ ID NO:31, SEQ ID NO:49, and SEQ ID NO:50.

FIG. 68 shows embodiments of a cloning strategy for CsOMT1. From top to bottom, the sequences shown are: SEQ ID NO:33, SEQ ID NO:51, and SEQ ID NO:52.

FIG. 69 shows embodiments of a cloning strategy for CsPT3. From top to bottom, the sequences shown are: SEQ ID NO:35, SEQ ID NO:53, and SEQ ID NO:54.

FIG. 70 shows an embodiment of the CsEPSPS prime editing mutation strategy as described herein.

FIG. 71 shows an embodiment of the CsEPSPS prime editing mutation strategy as described herein.

FIG. 72 shows the phenotype of T0 transgenic 3WS Cannabis plant WP-001181-1b (“Hernanda”) derived from particle-mediated transformation of meristem explants after transplant to large pot.

FIG. 73 shows Cannabis WP001181-1b particle gun T0 event aadA1a PCR.

FIG. 74 shows Cannabis WP001181-1b particle gun T0 event GUS expression.

FIG. 75 shows a Cannabis seed (Badger variety) surface sanitized followed by incubation at 37° C. without imbibition (note radical emergence in seed on left).

FIG. 76 shows the phenotype of Cannabis meristem explants of Badger variety after 3-day co-culture.

FIG. 77 shows transient GUS expression in Cannabis meristem explants of Badger variety after 3-day co-culture.

FIG. 78 shows stable RFP (tdTomato) expression in Cannabis meristem explants (variety Badger).

FIG. 79 shows a process diagram timeline for Cannabis sativa Agrobacterium-mediated transformation of embodiments described herein. Stage 1 shows Cannabis seeds. Stage 2 shows appearance of Cannabis meristem explant (mature embryo) derived from imbibed seed, the explant on left has cotyledonary tissue intact; explant on right has cotyledonary tissue removed. Stage 3 shows transient GUS activity in Honey Gold 3WS Cannabis explants after co-culture with Agrobacterium. Stage 4 shows appearance of Cannabis meristem explants after 4 day co-culture. Stage 5 shows phenotypes of Cannabis meristem explants after ˜3 weeks on 50 mg/L spectinomycin; note incomplete bleaching. Stage 6 shows Cannabis T0 event after second selection on 50 mg/L streptomycin prior to handoff to greenhouse. Finally, Stage 7 shows Cannabis T0 event prior to transplant in greenhouse.

FIG. 80 shows a process diagram timeline for Cannabis sativa Particle-mediated transformation. Stage 1 shows Cannabis seeds. Stage 2 shows appearance of Cannabis meristem explant (mature embryo) derived from imbibed seed, the explant on left has cotyledonary tissue intact; explant on right has cotyledonary tissue removed. Stage 3 shows Cannabis embryo target on CMC targeting media. Stage 4 shows PDS-1000 Helium gun. Stage 5 shows transient GUS activity in Honey Gold 3WS Cannabis explants 1-day post-bombardment. Stage 6 shows phenotypes of Cannabis meristem explants 2 months post-bombardment. Stage 7 shows Cannabis T0 event prior to handoff to greenhouse. Finally, Stage 8 shows Cannabis T0 event prior to transplant in greenhouse.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION OF THE INVENTION

The present disclosure describes methods for preparation and transformation of meristem explants from Cannabis. The meristem explant preparation methods described herein allow for pretreatment of the tissues for higher explant transformation and longer explant storage following excision.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

Provided herein are methods for preparing an explant suitable for transformation from a Cannabis seed. Also provided herein are methods of transforming and gene editing Cannabis meristem explant tissue. The explants generated by the methods described herein exhibit higher transformation efficiency with a broader capacity to customize the transformation process via pretreatment of the meristematic tissue used to generate the explants. The methods described herein allow for high scale production of storable explants for more effect transformation methods. As described in further detail below, the protocols described herein allow for targeted pretreatment of the meristematic tissues used in explant preparation at various stages and with various factors to improve explant storage and transformation efficiency.

As used herein, “embryo” refers to part of a seed, consisting of precursor tissues (meristematic tissues) for the leaves, stem, and root, as well as one or more cotyledons. Once the embryo begins to grow (germinate), it becomes a seedling plant.

As used herein, “meristem” or “meristematic tissue” refers to the portion of a seed that consists of undifferentiated cells, the meristematic cells, which differentiate to produce multiple plant structures including stem, roots, leaves, germline tissues and seeds. The meristematic cells are the targets for transformation to obtain transgenic plants.

As used herein, “explant” refers to the target material for transformation.

As used herein, “germline transformation” refers to the transformation of a gene of interest into cells that give rise to pollen or ovule thus into seed.

In a first aspect, provided herein is a method for preparing an explant from the meristematic tissue of a seed, where the method generally comprises the steps of drying the seed, surface sterilizing the seed, imbibing the seed until sufficiently hydrated, excising meristematic tissue from the hydrated seed to generate an explant, and optionally drying the excised meristematic tissue to generate the storable explant for transformation. The explants generated by the methods described herein are suitable for use in any transformation method known in the art.

The methods described herein also include one or more priming steps in which one or more priming agents are added to either the hydration medium during imbibing of the seed or to the explant as it is drying to generate a value added explant. As used herein, the term “value added explant” refers to an explant prepared by the methods described herein when a priming factor has been included in the hydration medium or a transformation supplement is included during drying of the explant.

The method includes a first step of drying a seed or acquiring a dried seed from which the explant will be generated. Preferably, a dry seed for use in the methods of the present invention will have a moisture content of between 1% and 25%. Ideally seeds are grown and harvested to achieve a viable embryo and are grown and harvested and cleaned to achieve blemish-free identity preserved seeds free of plant diseases and microbes that could interfere with sterile tissue culture. It may be desirable to treat the plants with fungicides and/or natural or synthetic plant regulators to improve embryo viability, embryo storage quality, seed coat intactness, seed vigor, percent germination cell response in tissue culture and transformation.

Seeds from which explants are to be prepared may be harvested from any Cannabis cultivar of interest. In some embodiments, the seed is from Cannabis sativa. In some embodiments, the seed is from Cannabis indica. In some embodiments, the seed is from a Cannabis variety developed from cross breeding of Cannabis sativa and Cannabis indica. In some embodiments, the seed is from a Cannabis sativa L. plant with less than 0.3 percent tetrahydrocannabinol (THC) based on the dry weight. In some embodiments, the Cannabis sativa cultivar is selected from the group consisting of Elektra x Chardonnay, Honey Gold 3WS (also referred to in the art at 3W1), Abacus, and Fiber Hemp.

In some embodiments of the present invention, the dry seed is surface sterilized. Any means known in the art for surface sterilization can be used. Suitable methods for surface sterilization may include, but are not limited to, exposure of the seed surface to radiation, UV light, oxidizing gasses, heat, plasma, disinfecting solvents and agents. In some embodiments, the seed is surface sterilized with a chemical agent such as sodium hypochlorite. In some embodiments, the seed is surface sterilized with an antibacterial or antifungal agent. In some embodiments, the seed is surface sterilized with ethanol (e.g., 20%, 30%, 40%, 50%, 60%, 70%, 80%, 90%, or 100% ethanol). In some embodiments, the seed is surface sterilized with Clorox™ bleach.

The dry seed, which in some embodiments has undergone surface sterilization, is imbibed under conditions that support hydration of the seed. The dry seed is hydrated in a hydration medium and for a time sufficient for the seed to reach a moisture content of between 30% and 70%. In some embodiments, the seed is hydrated for at least 12 hours. In some embodiments, the seed is hydrated between 2 and 24 hours.

The hydration medium used for hydration of the seed maybe any suitable sterile hydration medium known in the art that supports survival of the meristematic tissue in the seed. In some embodiments, the hydration medium is a modified sterile water and includes antibiotics or antifungals. In some embodiments, the hydration medium is a tissue culture medium that includes natural or synthetic plant growth regulators, plant tissue culture nutrients, a carbon source or a non-nutritive osmoregulator. In one embodiment, the hydration medium is bean germination medium, which includes the components outlined in Table 1 of Example 1.

In some embodiments of the invention, the hydration medium may optionally include one or more priming factors for pretreatment of the meristematic tissue. As used herein, “priming factor” or “priming agent” references to any molecule or substance included in the hydration medium which promotes survival and storage of the prepared explant or that promotes or increases the transformation efficiency of the prepared explant. Priming factors for use in the hydration medium of the present invention may include, but are not limited to, small molecules, biological molecules such as nucleic acids, polypeptides, proteins, antibodies, transcription factors, and macromolecules or complexes thereof, nanoparticles, liposomes, and cell-penetrating peptides. In some embodiments, the priming factor is a plant growth factor including, but not limited to, thidiazuron (TDZ), 6-benzylaminopurine (BAP), polyethylene glycol (PEG), 2,4-dichlorophenoxyacetic acid (2,4-D), Paczol™, gibberellic acid (GA3), indole-3-acetic acid (IAA), indole-3-butyric acid (IBA), 1-naphthalaneacetic acid (NAA), forchlorfenuron (CPPU), spectinomycin, streptomycin, glyphosate, glufosinate, bialophos, hygromycin, amikacin, tobramycin, imazapyr, dicamba, polyvinylpyrrolidone (PVP), polyvinylpolypyrrolidone (PVPP), salicylic acid, proline, betaine, ethylene, brassinosteroids, nitrates, and gibberellins.

Following hydration of the seed, meristematic tissue is excised to form an explant. Excision of the meristematic tissue may be performed by any means known in the art in which the seed coat and cotyledons are removed from the seed. Suitable methods for the excision of the meristematic tissue may include, but are not limited to manual processing, wet milling using a series of rollers and spray nozzles, adjustable grinding plates, rods, knives, wheels and other mechanical or machine based excision methods. These may be composed of, but are not limited to, ceramics, metals, and synthetic polymers. Induced pressure, injected gasses, vacuum and turbulence are also suitable methods. Hydrated explants may be stored in suitable storage medium for up to 7 days. Suitable storage medium for the hydrated explants may be any medium that supports survival and competence of the explant tissue. In some embodiments, the explants are stored with an excess of liquid to explants by volume. In some embodiments, the storage medium is liquid medium including MS salts.

In some embodiments, the meristematic tissue is excised from a dry seed to form an explant without first imbibing the seed. A dry seed suitable for dry excision will typically have a moisture content of between 1% and 25%. Excision of the meristematic tissue from the dry seed may be performed by any means known in the art in which the seed coat and cotyledons are removed from the seed. Suitable methods for the excision of the meristematic tissue may include, but are not limited to manual processing, wet milling using a series of rollers and spray nozzles, adjustable grinding plates, rods, knives, wheels and other mechanical or machine based excision methods. These may be composed of, but are not limited to, ceramics, metals, and synthetic polymers. Induced pressure, injected gasses, vacuum and turbulence are also suitable methods.

Following excision, the explant may be dried. Desiccation of the explant may be performed by any means known in the art such that the moisture content of the dry explant is between 1% and 25%. Suitable methods for desiccating the explant may include, but are not limited to, drying in the presence of air with and without an added dehumidifying agent. In some embodiments, the explants are dried in a laminar flow hood. In some embodiments, the explants are dried on the surface of filter paper in a laminar flow hood for about 26 hours. In some embodiments, the explants are dried in a dehumidifier. In some embodiments, the explants are dried at a temperature between 0° C. and 35° C. for at least 5 hours (e.g., at least 5, 7, 9, 12, 15, 18, 24, 30, 36, 42, 48, 72, 96 or 120 hours) and up to 2 weeks (e.g., up to 1, 2, 3, 4, 5, 6, 7, 8, 9, 10, 11, 12, 13, or 14 days). In some embodiments, it may be beneficial to control rates of drying by tightly controlling temperature, humidity, air flow, and time.

During desiccation of the explant, one or more transformation supplements may be added. As used herein, “transformation supplement” refers to any molecule or substance added to the explant during desiccation, which promotes survival and storage of the prepared explant or that promotes or increases the transformation efficiency of the prepared explant. Transformation supplements for use during desiccation of the explant of the present invention may include small molecules, biological molecules such as nucleic acids, polypeptides, proteins, antibodies, transcription factors, and macromolecules or complexes thereof, nanoparticles, liposomes, Agrobacterium, Rhizobium, and cell-penetrating peptides. In some embodiments, the transformation supplement is a plant growth factor, cell protectant agent including, or other agent including, but not limited to, thidiazuron (TDZ), 6-benzylaminopurine (BAP), polyethylene glycol (PEG), alginates and alginate complexes, starches, celluloses, synthetic polymers, gums, waxes, proline, betaine, polyvinylpyrrolidone (PVP), polyvinylpolypyrrolidone (PVPP), salicylic acid, calcium sources, silicone sources, colchicine, 2,4-dichlorophenoxyacetic acid (2,4-D), Paczol™, gibberellic acid (GA3), gibberellin (GA) pathway inhibitors, indole-3-acetic acid (IAA), indole-3-butyric acid (IBA), 1-naphthalaneacetic acid (NAA), forchlorfenuron (CPPU), spectinomycin, streptomycin, glyphosate, glufosinate, bialophos, hygromycin, amikacin, tobramycin, imazapyr, lyophilized agrobacterium, lyophilized rhizobium, and potassium hydroxide (KOH). In some embodiments, the transformation supplement is an agent, which promotes multiplication of the meristematic tissue, such as, but not limited to, TDZ, BAP, zeatin, kinetin, and CPPU. In some embodiments, explants are mechanically wounded prior to drying and storage. This can be achieved with exposure to ultrasound energy (e.g., sonication), liquid nitrogen, centrifugation, pressure, and chemical (ex. KOH, PEG, acids, bases), enzymes, abrasives, water jets, lasers, needles, or blades.

The dried explants are suitable for storage in a variety of conditions. Dried explants may be stored at temperatures ranging from about −200° C. to 50° C. (i.e., about −190° C. to 40° C., about −170° C. to 30° C., about −150° C. to 20° C., about −130° C. to 10° C., and about −102° C. to 0° C.) for a period of time of at least 7 days (i.e., at least 10 days, at least 30 days, at least 50 days, at least 60 days, at least 75 days, at least 90 days, and at least 120 days). Storable dried explants can also be banked to create libraries of germplasms from a variety of cultivars of agronomic significance. In general, lower temperature storage under dry conditions or controlled humidity will allow for prolonged storage of the dried Cannabis explants.

Explants generated by the methods described herein can be transformed with a heterologous gene or nucleic acid of interest by any means known in the art. Various methods have been developed for transferring genes or nucleic acids into plant tissue including particle bombardment, high velocity microprojection, microinjection, electroporation, direct DNA uptake, silica carbide fibers, cell-penetrating peptides, nanoparticles, viral vectors, and bacterially-mediated transformation. Bacteria known to mediate plant cell transformation include a number of species of the Rhizobiaceae, including, but not limited to, Agrobacterium spp., Sinorhizobium spp., Mesorhizobium spp., Rhizobium spp., Ochrobacterium spp., and Bradyrhizobium spp. In some embodiments, the explant is transformed using Agrobacterium spp. In some embodiments, the explant is transformed using Agrobacterium strain Ar18r12v. In some embodiments, the explant is transformed using particle bombardment using gold microcarriers. Suitable viral vectors are known and described in the art and may include, but are not limited to, tomato yellow leaf curl virus (TYLCV), tobacco yellow dwarf virus (TobYDV), tomato golden mosaic virus (TGMV), bean pod mottle virus (BPMV). Suitable methods of plant transformation are described in the art, such as, for example, by McCabe et al. (McCabe, D. E., Swain, W. F., Martinell, B. J., Christou, P. (1988) Nature Biotechnology 6(8), 923-926), Chen et al. (Chen, Y., Rivlin, A. Lange, A., Ye, X., Vaghchhipawala, Z., Eisinger, E., Dersch, E., Paris, M., Martinell, B., Wan, Y. (2014) Plant Cell Reports 33(1), 153-164), Ye et al. (Ye, X., Williams, E. J., Shen, J., Johnson, S., Lowe, B., Radke, S., Strickland, S. Esser, J. A., Petersen, M. W., and Gilbertson, L. A. (2011) Transgenic Research 20(4), 773-7860), and Plant Transformation Technologies (Edited by C. Neal Stewart, Alisher Touraev, Vitaly Citovsky and Tzvi Tzfira® 2011 Blackwell Publishing Ltd. ISBN: 978-0-813-82195-5.)

Prior to inoculation, dried explants to be transformed may be rehydrated using suitable tissue culture medium. In some embodiments, the rehydration medium is Soy INO medium, VAE rehydration medium, or a solid medium such as Basal MS or Gamborg's B5 medium. In some embodiments, the rehydration step is combined with the pretreatment step described below.

Embryos may be pretreated prior to inoculation and transformation. In some embodiments, the embryos are pretreated with a polyethylene glycol (PEG) solution. In some embodiments, the PEG solution includes about 20% PEG4000. In some embodiments, the embryos are pretreated in the PEG-ethanol solution for between about 1 minute and about 3 hours (e.g. 1min., 2 min., 5 min., 15 min., 30 min., 45 min., 1 hr., 1.5 hr., 2 hr., 2.5 hr., or 3 hr.). In some embodiments, salts may be added to the PEG solution. In some embodiments, the PEG solution includes one or more fungicides (e.g., Captan, Bravo, etc.). In some embodiments, the PEG solution includes Murashige and Skoog (MS) salts. In some embodiments, the pretreatment step includes sonication, vortexing, centrifugation, heat-shock, or addition of chemicals (e.g., TDZ, glyphosate, or metolachlor).

In some embodiments, the explant transformation is supplemented with force treatments. Force treatments may include, but are not limited to, sonication, vortexing, centrifugation, increased pressure, vacuum infiltration, heat-shock, dessication or addition of chemicals (e.g., TDZ, glyphosate, or metolachlor). Force treatments may be applied prior to or during transformation. For example, prior to or concurrently with inoculation with Agrobacterium or particle bombardment treatment. In some embodiments, explants are sonicated for about 20 seconds at about 45 kHz. Force treatment transformation methods are described in the art. See, for example, Khanna, et al. (“Centrifugation assisted Agrobacterium tumefaciens-mediated transformation (CAAT) of embryogenic cell suspensions of banana (Musa spp. Cavendish AAA and Lady finger AAA),” Molecular Breeding, 2004, 14:239-252), Kapila et al. (“An Agrobacterium-mediated transient gene expression system for intact leaves,” Plant Science, 1997, 122(1):101-108), Trick et al. (“SAAT: sonication-assisted Agrobacterium-mediated transformation,” Transgenic Research, 1997, 6(5):329-336), and Hiei et al. (“Improved frequency of transformation in rice and maize by treatment of immature embryos with centrifugation and heat prior to infection with Agrobacterium tumefaciens,” Plant Cell, Tissue, and Organ Culture, 2006, 87(3): 233-243).

Following inoculation, the explants are co-cultured for between about 1 day and about 6 days in any suitable co-culture medium that supports the growth and survival of the inoculated explant. In some embodiments, the co-culture medium is WCIC INO medium, which includes the components outlined in Table 2 of Example 1.

Following transformation, the explants or transformed tissue is regenerated using a suitable regeneration medium that supports the growth and survival of at least the positively transformed explants or transformed tissues. Suitable regeneration medium are known and described in the art. For example, the regeneration medium may be B5 medium, WPM based medium, MS salts based medium, 1/2X MS salts based medium, or similar medium with or without supplementation. The regeneration medium may additionally comprise nystatin, TBZ, meta-topolin (mT), napthylacetic acid (NAA) and GA3. In some embodiments, the regeneration medium includes a selection agent to select for positive transformants. Examples of suitable selection agents include, but are not limited to, RFP, GUS, aadA1a, spectinomycin, streptomycin, and imazapyr.

The heterologous gene or nucleic acid of interest may be any gene or nucleic acid that may confer a particular desirable trait or phenotype in the transformed plant. Examples of suitable genes of agronomic interest envisioned by the present invention would include but are not limited to genes for disease, insect, or pest tolerance, herbicide tolerance, genes for quality improvements such as yield, nutritional enhancements, environmental or stress tolerances, or any desirable changes in plant physiology, growth, development, morphology or plant product(s) including starch production, modified oils production, high oil production, modified fatty acid content, high protein production, fruit ripening, enhanced animal and human nutrition, and biopolymers production. Also environmental stress resistance, pharmaceutical peptides and secretable peptides, improved processing traits, industrial enzyme production, improved flavor, nitrogen fixation, hybrid seed production, and fiber production. Any of these or other genetic elements, methods, and transgenes may be used with the invention as will be appreciated by those of skill in the art in view of the instant disclosure. The heterologous gene or nucleic acid of interest may also be a sequence that can affect a phenotype of interest by encoding an RNA molecule that causes the targeted inhibition of expression on an endogenous gene via gene silencing technologies.

In some embodiments, the heterologous nucleic acid of interest modulates the expression or function of the endogenous Cannabis tetrahydrocannabinolic acid synthase (THCA synthase) gene. A heterologous nucleic acid is introduced into the Cannabis explant to knockout or silence the THCA synthase gene. Cannabis plants grown from the transformed Cannabis explant are characterized by a tetrahydrocannabinol (THC) low or THC free phenotype. The sequence and activity of the THCA synthase gene is known and described in the art. See, for example, Laverty et al. (Laverty et al., “A physical and genetic map of Cannabis sativa identifies extensive rearrangements at the THC/CBD acid synthase loci,” Genome Research, 2018, 29:146-156).

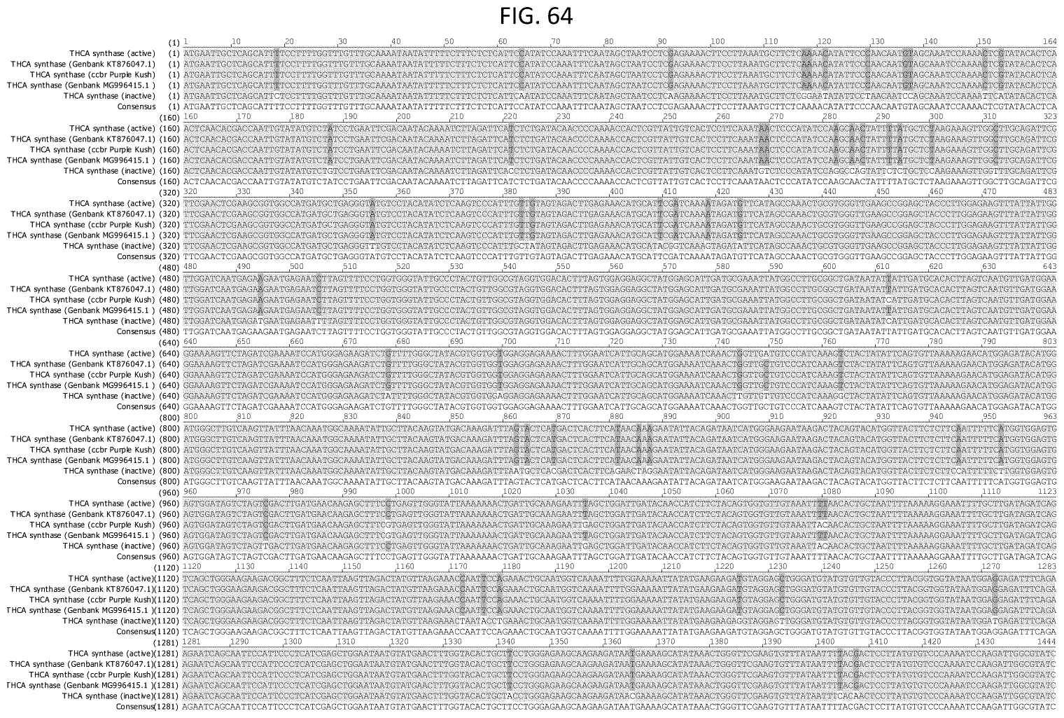

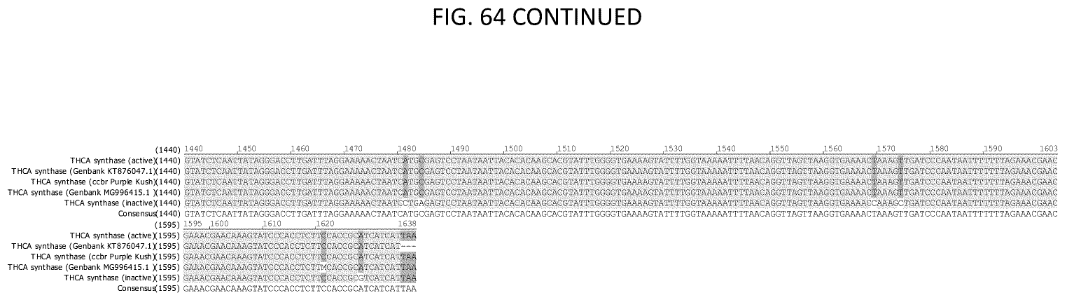

Provided below are five cDNA sequences (SEQ ID NOs:1-5) of THCA genes from various Cannabis sativa strains. Single nucleotide polymorphs (SNPs) between the active THCA synthase of SEQ ID NO:1 and each of SEQ ID NOs:2-5 are indicated in each of the respective sequences and a full sequence alignment is shown in FIG. 64.

Cannabis sativa cultivar Skunk #1 tetrahydrocannabinolic acid synthase (THCA1) gene
cDNA (SEQ ID NO: 1), Genbank: KJ469378.1
ATGAATTGCTCAGCATTTTCCTTTTGGTTTGTTTGCAAAATAATATTTTTCTTTCTCTCATTCC
ATATCCAAATTTCAATAGCTAATCCTCGAGAAAACTTCCTTAAATGCTTCTCAAAACATATTC
CCAACAATGTAGCAAATCCAAAACTCGTATACACTCAACACGACCAATTGTATATGTCTATC
CTGAATTCGACAATACAAAATCTTAGATTCATCTCTGATACAACCCCAAAACCACTCGTTAT
TGTCACTCCTTCAAATAACTCCCATATCCAAGCAACTATTTTATGCTCTAAGAAAGTTGGCTT
GCAGATTCGAACTCGAAGCGGTGGCCATGATGCTGAGGGTATGTCCTACATATCTCAAGTCC
CATTTGTTGTAGTAGACTTGAGAAACATGCATTCGATCAAAATAGATGTTCATAGCCAAACT
GCGTGGGTTGAAGCCGGAGCTACCCTTGGAGAAGTTTATTATTGGATCAATGAGAAGAATG
AGAATCTTAGTTTTCCTGGTGGGTATTGCCCTACTGTTGGCGTAGGTGGACACTTTAGTGGAG
GAGGCTATGGAGCATTGATGCGAAATTATGGCCTTGCGGCTGATAATATTATTGATGCACAC
TTAGTCAATGTTGATGGAAAAGTTCTAGATCGAAAATCCATGGGAGAAGATCTGTTTTGGGC
TATACGTGGTGGTGGAGGAGAAAACTTTGGAATCATTGCAGCATGGAAAATCAAACTGGTT
GATGTCCCATCAAAGTCTACTATATTCAGTGTTAAAAAGAACATGGAGATACATGGGCTTGT
CAAGTTATTTAACAAATGGCAAAATATTGCTTACAAGTATGACAAAGATTTAGTACTCATGA
CTCACTTCATAACAAAGAATATTACAGATAATCATGGGAAGAATAAGACTACAGTACATGG
TTACTTCTCTTCAATTTTTCATGGTGGAGTGGATAGTCTAGTCGACTTGATGAACAAGAGCTT
TCCTGAGTTGGGTATTAAAAAAACTGATTGCAAAGAATTTAGCTGGATTGATACAACCATCT
TCTACAGTGGTGTTGTAAATTTTAACACTGCTAATTTTAAAAAGGAAATTTTGCTTGATAGAT
CAGCTGGGAAGAAGACGGCTTTCTCAATTAAGTTAGACTATGTTAAGAAACCAATTCCAGAA
ACTGCAATGGTCAAAATTTTGGAAAAATTATATGAAGAAGATGTAGGAGCTGGGATGTATG
TGTTGTACCCTTACGGTGGTATAATGGAGGAGATTTCAGAATCAGCAATTCCATTCCCTCATC
GAGCTGGAATAATGTATGAACTTTGGTACACTGCTTCCTGGGAGAAGCAAGAAGATAATGA
AAAGCATATAAACTGGGTTCGAAGTGTTTATAATTTTACGACTCCTTATGTGTCCCAAAATCC
AAGATTGGCGTATCTCAATTATAGGGACCTTGATTTAGGAAAAACTAATCATGCGAGTCCTA
ATAATTACACACAAGCACGTATTTGGGGTGAAAAGTATTTTGGTAAAAATTTTAACAGGTTA
GTTAAGGTGAAAACTAAAGTTGATCCCAATAATTTTTTTAGAAACGAACAAAGTATCCCACC
TCTTCCACCGCATCATCATTAA
Cannabis sativa isolate 60D2 tetrahydrocannabinolic acid synthase (THCA) gene (SEQ
ID NO:2), GenBank: MG996415.1, single nucleotide polymorph (SNP) relative to SEQ ID NO: 1
shown in bold underline.
atgaattgctcagcattttccttttggtttgtttgcaaaataatatttttctttctctcattccatatccaaatttcaatagctaatcctcgagaaaacttc
cttaaatgcttctcaaaacatattcccaacaatgtagcaaatccaaaactegtatacactcaacacgaccaattgtatatgtctatcctgaattcg
acaatacaaaatcttagattcatctctgatacaaccccaaaaccactcgttattgtcactccttcaaataactcccatatccaagcaactattttat
gctctaagaaagttggcttgcagattcgaactcgaagcggtggccatgatgctgagggtatgtcctacatatctcaagtcccatttgttgtagta
gacttgagaaacatgcattcgatcaaaatagatgttcatagccaaactgcgtgggttgaagccggagctacccttggagaagtttattattgga
tcaatgagaagaatgagaatcttagttttcctggtgggtattgccctactgttggcgtaggtggacactttagtggaggaggctatggagcatt
gatgcgaaattatggccttgcggctgataatattattgatgcacacttagtcaatgttgatggaaaagttctagatcgaaaatccatgggagaa
gatctgttttgggctatacgtggtggtggaggagaaaactttggaatcattgcagcatggaaaatcaaactggttgctgtcccatcaaagtcta
ctatattcagtgttaaaaagaacatggagatacatgggcttgtcaagttatttaacaaatggcaaaatattgcttacaagtatgacaaagatttag
tactcatgactcacttcataacaaagaatattacagataatcatgggaagaataagactacagtacatggttacttctcttcaatttttcatggtgg
agtggatagtctagtcgacttgatgaacaagagctttcgtgagttgggtattaaaaaaactgattgcaaagaatttagctggattgatacaacca
tcttctacagtggtgttgtaaattttaacactgctaattttaaaaaggaaattttgcttgatagatcagctgggaagaagacggctttctcaattaag
ttagactatgttaagaaaccaattccagaaactgcaatggtcaaaattttggaaaaattatatgaagaagatgtaggagctgggatgtatgtgtt
gtacccttacggtggtataatggaggagatttcagaatcagcaattccattccctcatcgagctggaataatgtatgaactttggtacactgcttc
ctgggagaagcaagaagataatgaaaagcatataaactgggttcgaagtgtttataattttacgactccttatgtgtcccaaaatccaagattg
gcgtatctcaattatagggaccttgatttaggaaaaactaatcatgcgagtcctaataattacacacaagcacgtatttggggtgaaaagtatttt
ggtaaaaattttaacaggttagttaaggtgaaaactaaagttgatcccaataatttttttagaaacgaacaaagtatcccacctcttmcaccgca
tcatcattaa
Cannabis sativa clone ABC67 THCA synthase gene (SEQ ID NO: 3), GenBank:
KT876047.1, SNPs relative to SEQ ID NO: shown in bold underline.
atgaattgctcagcattttccttttggtttgtttgcaaaataatatttttctttctctcattccatatccaaatttcaatagctaatcctcgagaaaacttc
cttaaatgcttctcaaaacatattcccaacaatgtagcaaatccaaaactcgtatacactcaacacgaccaattgtatatgtctatcctgaattcg
acaatacaaaatcttagattcatctctgatacaaccccaaaaccactcgttattgtcactccttcaaataactcccatatccaagcaactattttat
gctctaagaaagttggcttgcagattcgaactcgaagcggtggccatgatgctgagggtatgtcctacatatctcaagtcccatttgttgtagta
gacttgagaaacatgcattcgatcaaaatagatgttcatagccaaactgcgtgggttgaagccggagctacccttggagaagtttattattgga
tcaatgagaagaatgagaatcttagttttcctggtgggtattgccctactgttggcgtaggtggacactttagtggaggaggctatggagcatt
gatgcgaaattatggccttgcggctgataatattattgatgcacacttagtcaatgttgatggaaaagttctagatcgaaaatccatgggagaa
gatctgttttgggctatacgtggtggtggaggagaaaactttggaatcattgcagcatggaaaatcaaactggttgctgtcccatcaaagtcta
ctatattcagtgttaaaaagaacatggagatacatgggcttgtcaagttatttaacaaatggcaaaatattgcttacaagtatgacaaagatttag
tactcatgactcacttcataacaaagaatattacagataatcatgggaagaataagactacagtacatggttacttctcttcaatttttcatggtgg
agtggatagtctagtcgacttgatgaacaagagctttcctgagttgggtattaaaaaaactgattgcaaagaatttagctggattgatacaacca
tcttctacagtggtgttgtaaattttaacactgctaattttaaaaaggaaattttgcttgatagatcagctgggaagaagacggctttctcaattaag
ttagactatgttaagaaaccaattccagaaactgcaatggtcaaaattttggaaaaattatatgaagaagatgtaggagctgggatgtatgtgtt
gtacccttacggtggtataatggaggagatttcagaatcagcaattccattccctcatcgagctggaataatgtatgaactttggtacactgcttc
ctgggagaagcaagaagataatgaaaagcatataaactgggttcgaagtgtttataattttacgactccttatgtgtcccaaaatccaagattg
gcgtatctcaattatagggaccttgatttaggaaaaactaatcatgcgagtcctaataattacacacaagcacgtatttggggtgaaaagtatttt
ggtaaaaattttaacaggttagttaaggtgaaaactaaagttgatcccaataatttttttagaaacgaacaaagtatcccacctcttccaccgcat
catcat
THCA synthase Cannabis sativa Purple Kush (SEQ ID NO: 4), SNPs relative to SEQ
ID NO: 1 shown in bold underline.
ATGAATTGCTCAGCATTTTCCTTTTGGTTTGTTTGCAAAATAATATTTTTCTTTCTCTC
ATTCCATATCCAAATTTCAATAGCTAATCCTCGAGAAAACTTCCTTAAATGCTTCTCA
AAACATATTCCCAACAATGTAGCAAATCCAAAACTCGTATACACTCAACACGACCA
ATTGTATATGTCTATCCTGAATTCGACAATACAAAATCTTAGATTCATCTCTGATACA
ACCCCAAAACCACTCGTTATTGTCACTCCTTCAAATAACTCCCATATCCAAGCAACT
ATTTTATGCTCTAAGAAAGTTGGCTTGCAGATTCGAACTCGAAGCGGTGGCCATGAT
GCTGAGGGTATGTCCTACATATCTCAAGTCCCATTTGTTGTAGTAGACTTGAGAAAC
ATGCATTCGATCAAAATAGATGTTCATAGCCAAACTGCGTGGGTTGAAGCCGGAGCT
ACCCTTGGAGAAGTTTATTATTGGATCAATGAGAAGAATGAGAATCTTAGTTTTCCT
GGTGGGTATTGCCCTACTGTTGGCGTAGGTGGACACTTTAGTGGAGGAGGCTATGGA
GCATTGATGCGAAATTATGGCCTTGCGGCTGATAATATCATTGATGCACACTTAGTC
AATGTTGATGGAAAAGTTCTAGATCGAAAATCCATGGGAGAAGATCTGTTTTGGGCT
ATACGTGGTGGTGGAGGAGAAAACTTTGGAATCATTGCAGCATGGAAAATCAAACT
GGTTGCTGTCCCATCAAAGTCTACTATATTCAGTGTTAAAAAGAACATGGAGATACA
TGGGCTTGTCAAGTTATTTAACAAATGGCAAAATATTGCTTACAAGTATGACAAAGA
TTTAGTACTCATGACTCACTTCATAACAAAGAATATTACAGATAATCATGGGAAGAA
TAAGACTACAGTACATGGTTACTTCTCTTCAATTTTTCATGGTGGAGTGGATAGTCTA
GTCGACTTGATGAACAAGAGCTTTCGTGAGTTGGGTATTAAAAAAACTGATTGCAA
AGAATTGAGCTGGATTGATACAACCATCTTCTACAGTGGTGTTGTAAATTACAACAC
TGCTAATTTTAAAAAGGAAATTTTGCTTGATAGATCAGCTGGGAAGAAGACGGCTTT
CTCAATTAAGTTAGACTATGTTAAGAAACCAATTCCAGAAACTGCAATGGTCAAAAT
TTTGGAAAAATTATATGAAGAAGATGTAGGAGCTGGGATGTATGTGTTGTACCCTTA
CGGTGGTATAATGGAGGAGATTTCAGAATCAGCAATTCCATTCCCTCATCGAGCTGG
AATAATGTATGAACTTTGGTACACTGCTTCCTGGGAGAAGCAAGAAGATAATGAAA
AGCATATAAACTGGGTTCGAAGTGTTTATAATTTTACGACTCCTTATGTGTCCCAAA
ATCCAAGATTGGCGTATCTCAATTATAGGGACCTTGATTTAGGAAAAACTAATCATG
CGAGTCCTAATAATTACACACAAGCACGTATTTGGGGTGAAAAGTATTTTGGTAAAA
ATTTTAACAGGTTAGTTAAGGTGAAAACTAAAGTTGATCCCAATAATTTTTTTAGAA
ACGAACAAAGTATCCCACCTCTTCCACCGCATCATCATTAA
Inactive THCA synthase gene (SEQ ID NO: 5), SNPs relative to SEQ ID NO: 1 shown
in bold underline.
ATGAATTGCTCAGCATTCTCCTTTTGGTTTGTTTGCAAAATAATATTTTTCTTTCTCTC
ATTCAATATCCAAATTTCAATAGCTAATCCTCAAGAAAACTTCCTTAAATGCTTCTC
GGAATATATTCCTAACAATCCAGCAAATCCAAAATTCATATACACTCAACACGACC
AATTGTATATGTCTGTCCTGAATTCGACAATACAAAATCTTAGATTCACCTCTGATA
CAACCCCAAAACCACTCGTTATTGTCACTCCTTCAAATGTCTCCCATATCCAGGCCA
GTATTCTCTGCTCCAAGAAAGTTGGTTTGCAGATTCGAACTCGAAGCGGTGGCCATG
ATGCTGAGGGTTTGTCCTACATATCTCAAGTCCCATTTGCTATAGTAGACTTGAGAA
ACATGCATACGGTCAAAGTAGATATTCATAGCCAAACTGCGTGGGTTGAAGCCGGA
GCTACCCTTGGAGAAGTTTATTATTGGATCAATGAGATGAATGAGAATTTTAGTTTT
CCTGGTGGGTATTGCCCTACTGTTGGCGTAGGTGGACACTTTAGTGGAGGAGGCTAT
GGAGCATTGATGCGAAATTATGGCCTTGCGGCTGATAATATCATTGATGCACACTTA
GTCAATGTTGATGGAAAAGTTCTAGATCGAAAATCCATGGGAGAAGATCTATTTTGG
GCTATACGTGGTGGAGGAGGAGAAAACTTTGGAATCATTGCAGCATGGAAAATCAA
ACTTGTTGTTGTCCCATCAAAGGCTACTATATTCAGTGTTAAAAAGAACATGGAGAT
ACATGGGCTTGTCAAGTTATTTAACAAATGGCAAAATATTGCTTACAAGTATGACAA
AGATTTAATGCTCACGACTCACTTCAGAACTAGGAATATTACAGATAATCATGGGA
AGAATAAGACTACAGTACATGGTTACTTCTCTTCCATTTTTCTTGGTGGAGTGGATA
GTCTAGTTGACTTGATGAACAAGAGCTTTCCTGAGTTGGGTATTAAAAAAACTGATT
GCAAAGAATTGAGCTGGATTGATACAACCATCTTCTACAGTGGTGTTGTAAATTACA
ACACTGCTAATTTTAAAAAGGAAATTTTGCTTGATAGATCAGCTGGGAAGAAGACG
GCTTTCTCAATTAAGTTAGACTATGTTAAGAAACTAATACCTGAAACTGCAATGGTC
AAAATTTTGGAAAAATTATATGAAGAAGAGGTAGGAGTTGGGATGTATGTGTTGTA
CCCTTACGGTGGTATAATGGATGAGATTTCAGAATCAGCAATTCCATTCCCTCATCG
AGCTGGAATAATGTATGAACTTTGGTACACTGCTACCTGGGAGAAGCAAGAAGATA
ACGAAAAGCATATAAACTGGGTTCGAAGTGTTTATAATTTCACAACTCCTTATGTGT
CCCAAAATCCAAGATTGGCGTATCTCAATTATAGGGACCTTGATTTAGGAAAAACTA
ATCCTGAGAGTCCTAATAATTACACACAAGCACGTATTTGGGGTGAAAAGTATTTTG
GTAAAAATTTTAACAGGTTAGTTAAGGTGAAAACCAAAGCTGATCCCAATAATTTTT
TTAGAAACGAACAAAGTATCCCACCTCTTCCACCGCGTCATCATTAA
THCA synthase polypeptide sequence (SEQ ID NO: 6)
MNCSAFSFWFVCKIIFFFLSFHIQISIANPRENFLKCFSKHIPNNVANPKLVYTQHDQLYM
SILNSTIQNLRFISDTTPKPLVIVTPSNNSHIQATILCSKKVGLQIRTRSGGHDAEGMSYISQ
VPFVVVDLRNMHSIKIDVHSQTAWVEAGATLGEVYYWINEKNENLSFPGGYCPTVGVG
GHFSGGGYGALMRNYGLAADNIIDAHLVNVDGKVLDRKSMGEDLFWAIRGGGGENFG
IIAAWKIKLVDVPSKSTIFSVKKNMEIHGLVKLFNKWQNIAYKYDKDLVLMTHFITKNIT
DNHGKNKTTVHGYFSSIFHGGVDSLVDLMNKSFPELGIKKTDCKEFSWIDTTIFYSGVV
NFNTANFKKEILLDRSAGKKTAFSIKLDYVKKPIPETAMVKILEKLYEEDVGAGMYVLY
PYGGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFRN
EQSIPPLPPHHH

In some embodiments, the THCA synthase gene may be knocked out using Clustered Randomly Interspersed Short Palindromic Repeats (CRISPR)/Cas mediated gene editing. To knockout the THCA synthase gene using CRISPR/Cas mediated gene editing, one or more guide RNAs are designed that target the THCA synthase gene or a region adjacent there to and proximal to a Protospacer Adjacent Motif (PAM) site. Upon introduction to a Cannabis cell, the guide RNAs target a nuclease to induce a double strand break at the designated cut site. The cell will then undergo non-homologous end joining (NHEJ) to repair the cut site. Due to the nature of NHEJ, one or more insertions or deletions (indels) are introduced at the cut site, thereby silencing the target THCA synthase gene. In some embodiments, a homology directed repair (HDR) template oligonucleotide may be used to direct the repair at the cut site to introduce a mutation of interest. The mutation of interest may be an insertion of a stop codon, a frameshift mutation, or a nonsense mutation that disrupts expression of the THCA synthase gene. In some embodiments, the HDR oligonucleotide encodes a sequence comprising one or more of the THCA synthase gene SNPs recited herein. In some embodiments, the guide RNAs direct cleavage of the THCA gene such that all or a portion of the THCA gene is removed.

In some embodiments, the nuclease is a Cas nuclease. Suitable Cas nucleases are known and described in the art including, but not limited to, a Cas9 nuclease.

In some embodiments, the guide RNA targeting the THCA gene is selected from SEQ ID NO:7 (TGCAGCATGGAAAATCAAAC, forward gRNA gRF743), SEQ ID NO:8 (CCCTTACGGTGGTATAATGG, forward gRNA gRF1271), SEQ ID NO:9 (TAGCTATTGAAATTTGGATA, reverse gRNA gRR63), SEQ ID NO:10

(TAGAGCATAAAATAGTTGCT, reverse gRNA gRR279), or combinations thereof. As used herein, the guide RNA (gRNA) nomenclature (e.g., gRF743) refers to the template direction of the gRNA, either forward (F) or reverse (R), and nucleotide position at which the 20 base pair (bp) gRNA ends. For example, gRF743 is a forward gRNA that aligns with the THCA synthase gene and ends at nucleotide 743. Other nomenclature schemes and identification methods will be known to a skilled artisan. In some embodiments, the guide RNA sequences are cloned into a vector for introduction to the cell. In some embodiments, the vector also encodes a Cas nuclease (e.g., Cas9 nuclease). In some embodiments, one or more vectors encoding the guide RNA and the Cas nuclease are introduced into the cell in the presence of an HDR oligonucleotide. Cells positive for transformation and the desired CRISPR/Cas mediated editing results may be screened and selected for using standard molecular biology and sequencing techniques known in the art.

In some embodiments, the heterologous nucleic acid of interest modulates the expression or function of the endogenous Cannabis cannabidiolic-acid synthase (CBDA synthase) gene. A heterologous nucleic acid is introduced into the Cannabis explant to knockout or silence the CBDA synthase gene. Cannabis plants grown from the transformed Cannabis explant are characterized by a tetrahydrocannabinol (THC) low or THC free phenotype. The sequence and activity of the CBDA synthase gene is known and described in the art. See, for example, Laverty et al. (Laverty et al., “A physical and genetic map of Cannabis sativa identifies extensive rearrangements at the THC/CBD acid synthase loci,” Genome Research, 2018, 29:146-156).

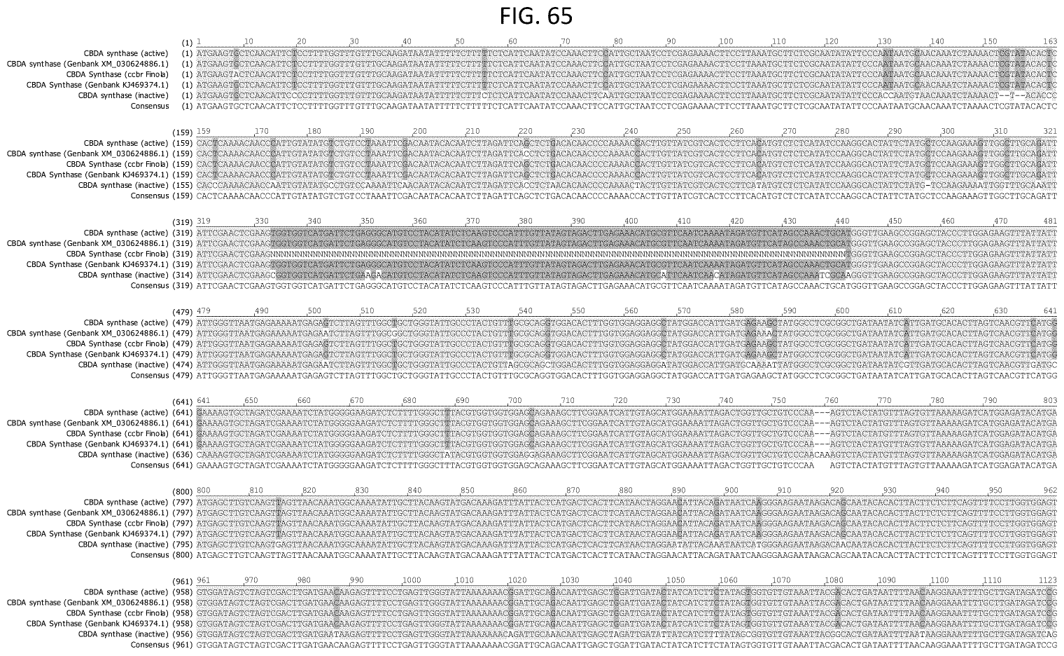

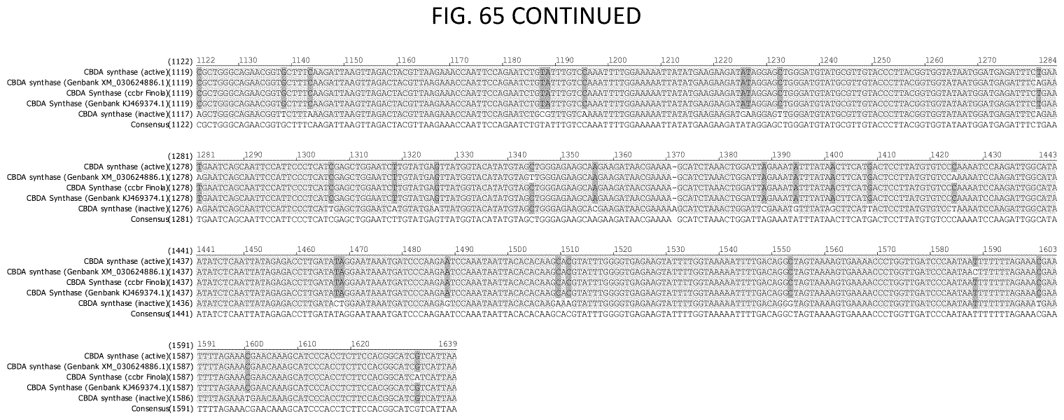

Provided below are four cDNA or reverse complement sequences (SEQ ID NOs:11, 13, 15, and 17) of CBDA genes from various Cannabis sativa strains. Single nucleotide polymorphs (SNPs) between the active CBDA synthase of SEQ ID NO:11 and each of SEQ ID NOs:13 and 15 are indicated in each of the respective sequences and a full sequence alignment is shown in FIG. 65. Additionally, mutations in the CBDA synthase sequences of SEQ ID NOs: 14 and 16 are shown relative to SEQ ID NO:12.

Cannabis sativa cultivar Carmen cannabidiolic acid synthase (CBDA1) gene (SEQ ID
NO: 11), GenBank: KJ469374.1
atgaagtgctcaacattctccttttggtttgtttgcaagataatatttttctttttctcattcaatatccaaacttccattgctaatcctcgagaaaact
tccttaaatgcttctcgcaatatattcccaataatgcaacaaatctaaaactcgtatacactcaaaacaacccattgtatatgtctgtcctaaattcga
caatacacaatcttagattcagctctgacacaaccccaaaaccacttgttatcgtcactccttcacatgtctctcatatccaaggcactattctatg
ctccaagaaagttggcttgcagattcgaactcgaagtggtggtcatgattctgagggcatgtcctacatatctcaagtcccatttgttatagtag
acttgagaaacatgcgttcaatcaaaatagatgttcatagccaaactgcatgggttgaagccggagctacccttggagaagtttattattgggtt
aatgagaaaaatgagagtcttagtttggctgctgggtattgccctactgtttgcgcaggtggacactttggtggaggaggctatggaccattga
tgagaagctatggcctcgcggctgataatatcattgatgcacacttagtcaacgttcatggaaaagtgctagatcgaaaatctatgggggaag
atctcttttgggctttacgtggtggtggagcagaaagcttcggaatcattgtagcatggaaaattagactggttgctgtcccaaagtctactatgt
ttagtgttaaaaagatcatggagatacatgagcttgtcaagttagttaacaaatggcaaaatattgcttacaagtatgacaaagatttattactcat
gactcacttcataactaggaacattacagataatcaagggaagaataagacagcaatacacacttacttctcttcagttttccttggtggagtgg
atagtctagtcgacttgatgaacaagagttttcctgagttgggtattaaaaaaacggattgcagacaattgagctggattgatactatcatcttct
atagtggtgttgtaaattacgacactgataattttaacaaggaaattttgcttgatagatccgctgggcagaacggtgctttcaagattaagttag
actacgttaagaaaccaattccagaatctgtatttgtccaaattttggaaaaattatatgaagaagatataggagctgggatgtatgcgttgtacc
cttacggtggtataatggatgagatttctgaatcagcaattccattccctcatcgagctggaatcttgtatgagttatggtacatatgtagctggg
agaagcaagaagataacgaaaagcatctaaactggattagaaatatttataacttcatgactccttatgtgtcccaaaatccaagattggcatat
ctcaattatagagaccttgatataggaataaatgatcccaagaatccaaataattacacacaagcacgtatttggggtgagaagtattttggtaa
aaattttgacaggctagtaaaagtgaaaaccctggttgatcccaataatttttttagaaacgaacaaagcatcccacctcttccacggcatcgtc
attaa
Cannabis sativa CBDA synthase (SEQ ID NO: 12)
MKCSTFSFWFVCKIIFFFFSFNIQTSIANPRENFLKCFSQYIPNNATNLKLVYTQNNPLYM
SVLNSTIHNLRFSSDTTPKPLVIVTPSHVSHIQGTILCSKKVGLQIRTRSGGHDSEGMSYIS
QVPFVIVDLRNMRSIKIDVHSQTAWVEAGATLGEVYYWVNEKNESLSLAAGYCPTVCA
GGHFGGGGYGPLMRSYGLAADNIIDAHLVNVHGKVLDRKSMGEDLFWALRGGGAESF
GIIVAWKIRLVAVPKSTMFSVKKIMEIHELVKLVNKWQNIAYKYDKDLLLMTHFITRNIT
DNQGKNKTAIHTYFSSVFLGGVDSLVDLMNKSFPELGIKKTDCRQLSWIDTIIFYSGVVN
YDTDNFNKEILLDRSAGQNGAFKIKLDYVKKPIPESVFVQILEKLYEEDIGAGMYALYPY
GGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLVKVKTLVDPNNFFRNEQSIP
PLPRHRH
Cannabis sativa cannabidiolic acid synthase (LOC115697762) (SEQ ID NO: 13),
GenBank: XM_030624886.1, SNPs relative to SEQ ID NO: 11 shown in bold underline.
ATGAAGTGCTCAACATTCTCCTTTTGGTTTGTTTGCAAGATAATATTTTTCTTTTTCTCATTCA
ATATCCAAACTTCCATTGCTAATCCTCGAGAAAACTTCCTTAAATGCTTCTCGCAATATATTC
CCAATAATGCAACAAATCTAAAACTCGTATACACTCAAAACAACCCATTGTATATGTCTGTC
CTAAATTCGACAATACACAATCTTAGATTCACCTCTGACACAACCCCAAAACCACTTGTTAT
CGTCACTCCTTCACATGTCTCTCATATCCAAGGCACTATTCTATGCTCCAAGAAAGTTGGCTT
GCAGATTCGAACTCGAAGTGGTGGTCATGATTCTGAGGGCATGTCCTACATATCTCAAGTCC
CATTTGTTATAGTAGACTTGAGAAACATGCGTTCAATCAAAATAGATGTTCATAGCCAAACT
GCATGGGTTGAAGCCGGAGCTACCCTTGGAGAAGTTTATTATTGGGTTAATGAGAAAAATGA
GAATCTTAGTTTGGCGGCTGGGTATTGCCCTACTGTTTGCGCAGGTGGACACTTTGGTGGAG
GAGGCTATGGACCATTGATGAGAAACTATGGCCTCGCGGCTGATAATATCATTGATGCACAC
TTAGTCAACGTTCATGGAAAAGTGCTAGATCGAAAATCTATGGGGGAAGATCTCTTTTGGGC
TTTACGTGGTGGTGGAGCAGAAAGCTTCGGAATCATTGTAGCATGGAAAATTAGACTGGTTG
CTGTCCCAAAGTCTACTATGTTTAGTGTTAAAAAGATCATGGAGATACATGAGCTTGTCAAG
TTAGTTAACAAATGGCAAAATATTGCTTACAAGTATGACAAAGATTTATTACTCATGACTCA
CTTCATAACTAGGAACATTACAGATAATCAAGGGAAGAATAAGACAGCAATACACACTTAC
TTCTCTTCAGTTTTCCTTGGTGGAGTGGATAGTCTAGTCGACTTGATGAACAAGAGTTTTCCT
GAGTTGGGTATTAAAAAAACGGATTGCAGACAATTGAGCTGGATTGATACTATCATCTTCTA
TAGTGGTGTTGTAAATTACGACACTGATAATTTTAACAAGGAAATTTTGCTTGATAGATCCG
CTGGGCAGAACGGTGCTTTCAAGATTAAGTTAGACTACGTTAAGAAACCAATTCCAGAATCT
GTATTTGTCCAAATTTTGGAAAAATTATATGAAGAAGATATAGGAGCTGGGATGTATGCGTT
GTACCCTTACGGTGGTATAATGGATGAGATTTCAGAATCAGCAATTCCATTCCCTCATCGAG
CTGGAATCTTGTATGAGTTATGGTACATATGTAGTTGGGAGAAGCAAGAAGATAACGAAAA
GCATCTAAACTGGATTAGAAATATTTATAACTTCATGACTCCTTATGTGTCCAAAAATCCAA
GATTGGCATATCTCAATTATAGAGACCTTGATATAGGAATAAATGATCCCAAGAATCCAAAT
AATTACACACAAGCACGTATTTGGGGTGAGAAGTATTTTGGTAAAAATTTTGACAGGCTAGT
AAAAGTGAAAACCCTGGTTGATCCCAATAACTTTTTTAGAAACGAACAAAGCATCCCACCTC
TTCCACGGCATCGTCATTAA

CBDA synthase protein sequence corresponding to SEQ ID NO:13 (SEQ ID NO:14), mutations relative to SEQ ID NO:12 shown in bold underline.

MKCSTFSFWFVCKIIFFFFSFNIQTSIANPRENFLKCFSQYIPNNATN
LKLVYTQNNPLYMSVLNSTIHNLRFTSDTTPKPLVIVTPSHVSHIQGT
ILCSKKVGLQIRTRSGGEIDSEGMSYISQVPFVIVDLRNMRSIKIDVH
SQTAWVEAGATLGEVYYVVVNEKNENLSLAAGYCPTVCAGGHFGGGG
YGPLMRNYGLAADNIIDAHLVNVHGKVLDRKSMGEDLFWALRGGGAES
FGIIVAWKIRLVAVPKSTMFSVKKIMEIHELVKLVNKWQNIAYKYDKD
LLLMTHFITRNITDNQGKNKTAIHTYFSSVFLGGVDSLVDLMNKSFPE
LGIKKTDCRQLSWIDTIIFYSGVVNYDTDNFNKEILLDRSAGQNGAFK
IKLDYVKKPIPESVFVQILEKLYEEDIGAGMYALYPYGGIMDEISESA
IPFPHRAGILYELWYICSWEKQEDNEKHLNVVIRNIYNFMTPYVSKNP
RLAYLNYRDLDIGINDPKNPNNYTQARIWGEKYFGKNFDRLVKVKTLV
DPNNFFRNEQSIPPLPRHRH

Cannabis sativa Finola CBDA synthase gene reverse complement (SEQ ID NO:15), SNPs relative to both SEQ ID NOs:11 and 13 shown in bold underline, SNPs relative to SEQ ID NO:13 shown in bold italics.

ATGAAGTACTCAACATTCTCCTTTTGGTTTGTTTGCAAGATAATATT
TTTCTTTTTCTCATTCAATATCCAAACTTCCATTGCTAATCCTCGAG
AAAACTTCCTTAAATGCTTCTCGCAATATATTCCCAATAATGCAACA
AATCTAAAACTCGTATACACTCAAAACAACCCATTGTATATGTCTGT
CCTAAATTCGACAATACACAATCTTAGATTCAGCTCTGACACAACC
CCAAAACCACTTGTTATCGTCACTCCTTCACATGTCTCTCATATCCA
AGGCACTATTCTATGCTCCAAGAAAGTTGGCTTGCAGATTCGAACTC
GAAGNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNN
NNNNNNNNNNNNNNNNNNNTGGGTTGAAGCCGGAGCTACCCTTGGAG
AAGTTTATTATTGGGTTAATGAGAAAAATGAGAGTCTTAGTTTGGCT
GCTGGGTATTGCCCTACTGTTTGCGCAGGTGGACACTTTGGTGGAGG
AGGCTATGGACCATTGATGAGAAGCTATGGCCTCGCGGCTGATAATA
TCATTGATGCACACTTAGTCAACGTTCATGGAAAAGTGCTAGATCGAA
AATCTATGGGGGAAGATCTCTTTTGGGCTTTACGTGGTGGTGGAGCAG
AAAGCTTCGGAATCATTGTAGCATGGAAAATTAGACTGGTTGCTGTCC
CAAAGTCTACTATGTTTAGTGTTAAAAAGATCATGGAGATACATGAGC
TTGTCAAGTTAGTTAACAAATGGCAAAATATTGCTTACAAGTATGACA
AAGATTTATTACTCATGACTCACTTCATAACTAGGAACATTACAGATA
ATCAAGGGAAGAATAAGACAGCAATACACACTTACTTCTCTTCAGTTT
TCCTTGGTGGAGTGGATAGTCTAGTCGACTTGATGAACAAGAGTTTTC
CTGAGTTGGGTATTAAAAAAACGGATTGCAGACAATTGAGCTGGATTG
ATACTATCATCTTCTATAGTGGTGTTGTAAATTACGACACTGATAATT
TTAACAAGGAAATTTTGCTTGATAGATCCGCTGGGCAGAACGGTGCTT
TCAAGATTAAGTTAGACTACGTTAAGAAACCAATTCCAGAATCTGTAT
TTGTCCAAATTTTGGAAAAATTATATGAAGAAGATATAGGAGCTGGGA
TGTATGCGTTGTACCCTTACGGTGGTATAATGGATGAGATTTCTGAAT
CAGCAATTCCATTCCCTCATCGAGCTGGAATCTTGTATGAGTTATGGT
ACATATGTAGCTGGGAGAAGCAAGAAGATAACGAAAAGCATCTAAACT
GGATTAGAAATATTTATAACTTCATGACTCCTTATGTGTCCCAAAATC
CAAGATTGGCATATCTCAATTATAGAGACCTTGATATAGGAATAAATG
ATCCCAAGAATCCAAATAATTACACACAAGCACGTATTTGGGGTGAGA
AGTATTTTGGTAAAAATTTTGACAGGCTAGTAAAAGTGAAAACCCTGG
TTGATCCCAATAATTTTTTTAGAAACGAACAAAGCATCCCACCTCTTC
CACGGCATCATCATTAA

CBDA synthase protein sequence corresponding to SEQ ID NO:15 (SEQ ID NO:16), mutations relative to both SEQ ID NOs: 12 and 14shown in bold underline, mutations relative to SEQ ID NO:14 shown in bold italics.

MKYSTFSFWFVCKIIFFFFSFNIQTSIANPRENFLKCFSQYIPNNATNLK
LVYTQNNPLYMSVLNSTIHNLRFSSDTTPKPLVIVTPSHVSHIQGTILCS
KKVGLQIRTRXXXXXXXXXXXXXXXXXXXXXKXXXXXXXXWVEAGATLGE
VYYWVNEKNESLSLAAGYCPTVCAGGHFGGGGYGPLMRSYGLAADNIIDA
HLVNVHGKVLDRKSMGEDLFWALRGGGAESFGIIVAWKIRLVAVPKSTMF
SVKKIMEIHELVKLVNKWQNIAYKYDKDLLLMTHFITRNITDNQGKNKTA
IHTYFSSVFLGGVDSLVDLMNKSFPELGIKKTDCRQLSWIDTIIFYSGVV
NYDTDNFNKEILLDRSAGQNGAFKIKLDYVKKPIPESVFVQILEKLYEED
IGAGMYALYPYGGIMDEISESAIPFPHRAGILYELWYICSWEKQEDNEKH
LNWIRNIYNFMTPYVSQNPRLAYLNYRDLDIGINDPKNPNNYTQARIWGE
KYFGKNFDRLVKVKTLVDPNNFFRNEQSIPPLPRHHH
Inactive CBDA synthase
(SEQ ID NO: 17)
ATGAAGTGCTCAACATTCCCCTTTTGGTTTGTTTGCAAGATAATATTTTT
CTTTCTCTCATTCAATATCCAAACTTCAATTGCTAATCCTCGAGAAAACT
TCCTTAAATGCTTCTCGCAATATATTCCCACCAATGTAACAAATCTAAAA
CTTACACCCAAAACAACCAATTGTATATGCCTGTCCAAAATTCAACAATA
CACAATCTTAGATTCACCTCTAACACAACCCCAAAACTACTTGTTATCGT
CACTCCTTCATATGTCTCTCATATCCAAGGCACTATTCTATGTCCAAGAA
AATTGGTTTGCAAATTCGAACTCGAAGCGGTGGTCATGATTCTGAAGACA
TGTCCTACATATCTCAAGTCCCATTTGTTATAGTAGACTTGAGAAACATG
CATTCAATCAACATAGATGTTCATAGCCAAATCGCAAGGGTTGAAGCCGG
AGCTACCCTTGGAGAAGTTTATTATTGGGTTAATGAGAAAAATGAGAATC
TTAGTTTGGCTGCTGGGTATTGCCCTACTGTTAGCGCAGCTGGACACTTT
GGTGGAGGAGGATATGGACCATTGATGCAAAATTATGGCCTCGCGGCTGA
TAATATCGTTGATGCACACTTAGTCAACGTTGATGCAAAAGTGCTAGATC
GAAAATCTATGGGGGAAGATCTCTTTTGGGCTATACGTGGTGGTGGAGGA
GAAAGCTTCGGAATCATTGTAGCATGGAAAATTAGACTGGTTGCTGTCCC
AACAAAGTCTACTATGTTTAGTGTTAAAAAGATCATGGAGATACATGAGC
TTGTCAAGTGAGTTAACAAATGGCAAAATATTGCTTACAAGTATGACAAA
GATTTATTACTCATGACTCACTTCATAACTAGGAATATTACAAATAATCA
TGGGAAGAATAAGACAACAATACACACTTACTTCTCTTCAGTTTTCCTTG
GTGGAGTGGATAGTCTAGTCGACTTGATGAATAAGAGTTTTCCTGAGTTG
GGTATTAAAAAAACAGATTGCAAACAATTGAGCTAGATTGATATTATCAT
CTTTTATAGCGGTGTTGTAAATTACGGCACTGATAATTTTAATAAGGAAA
TTTTGCTTGATAGATCAGCTGGGCAGAACGGTTCTTTAAAGATTAAGTTA
GACTACGTTAAGAAACCAATTCCAGAATCTGCGTTTGTCAAAATTTTGGA
AAAATTATATGAAGAAGATGAAGGAGTTGGGATGTATGCGTTGTACCCTT
ACGGTGGTATAATGGATGAGATTTCAGAATCAGCAATTCCATTCCCTCAT
TGAGCTGGAATCATGTATGAATTATGGTACATATGTAGCTGGGAGAAGCA
CGAAGATAACGAAAAAGCATCTAAACTGGATTCGAAATGTTTATAGCTTC
ATTACTCCTTATGTGTCCTAAAATCCAAGATTGGCATATCTCAATTATAG
AGACCTTGATACTGGAATAAATGATCCCAAGAGTCCAAATAATTACACAC
AAGAAAGTATTTGGGGTGAGAAGTATTTTGGTAAAAATTTTGACAGGGTA
GTAAAAGTGAAAACCCTGGTTGATCCCAATAATTTTTTTAGAAATGAACA
AAGCATCCCACCTCTTCCACGGCATCGTCATTAA

In some embodiments, the CBDA synthase gene may be knocked out using CRISPR/Cas mediated gene editing. To knockout the CBDA synthase gene using CRISPR/Cas mediated gene editing, one or more guide RNAs are designed that target the CBDA synthase gene or a region adjacent there to and proximal to a PAM site. Upon introduction to a Cannabis cell, the guide RNAs target a nuclease to induce a double strand break at the designated cut site. The cell will then undergo NHEJ to repair the cut site. Due to the nature of NHEJ, one or more indels are introduced at the cut site, thereby silencing the target CBDA synthase gene. In some embodiments, an HDR template oligonucleotide may be used to direct the repair at the cut site to introduce a mutation of interest. The mutation of interest may be an insertion of a stop codon, a frameshift mutation, or a nonsense mutation that disrupts expression of the CBDA synthase gene. In some embodiments, the HDR oligonucleotide encodes a sequence comprising one or more the CBDA synthase gene SNPs recited herein or encodes a mutation in the CBDA synthase polypeptide sequence as demonstrated herein. In some embodiments, the guide RNAs direct cleavage of the CBDA gene such that all or a portion of the CBDA gene is removed. In some embodiments, the nuclease is a Cas nuclease. Suitable Cas nucleases are known and described in the art including, but not limited to, a Cas9 nuclease.

In some embodiments, the guide RNA targeting the CBDA gene is selected from SEQ ID NO:18 (GCTAGATCGAAAATCTATGG, forward gRNA gRF668), SEQ ID NO:19 (AAAGCATCCCACCTCTTCCA, forward gRNA gRF1621), SEQ I NO: 20 (TTTAGGACAGACATATACAA, reverse gRNA gRR172), SEQ ID NO:21 (GAAAGCACCGTTCTGCCCAG, reverse gRNA gRR1118), or combinations thereof. In some embodiments, the guide RNA sequences are cloned into a vector for introduction to the cell. In some embodiments, the vector also encodes a Cas nuclease (e.g., Cas9 nuclease). In some embodiments, one or more vectors encoding the guide RNA and the Cas nuclease are introduced into the cell in the presence of an HDR oligonucleotide. Cells positive for transformation and the desired CRISPR/Cas mediated editing results may be screened and selected for using standard molecular biology and sequencing techniques known in the art.

In some embodiments, both the THCA synthase and CBDA synthase genes are knocked out to produce a THC low or THC free Cannabis plant. The polypeptide sequence of the THCA synthase and CBDA synthase enzymes are approximately 84% identical and both contribute to THC production in Cannabis. Plants produced by knocking out both the THCA synthase and CBDA synthase genes have significantly increased production of cannabigerolic acid (CBG), which is the substrate for both THCA synthase and CBDA synthase, relative to a Cannabis plant with wild-type expression of THCA synthase and CBDA synthase. In some embodiments, both the THCA synthase and the CBDA synthase genes are knocked-out using CRISPR/Cas mediated gene editing as described herein. In some embodiments, guide RNAs targeting both the THCA synthase gene and the CBDA gene simultaneously are designed and used for CRISPR/Cas9 mediated gene editing. In some embodiments, the guide RNAs targeting both THCA synthase and CBDA synthase are selected from SEQ ID NO:22 (CATTTAAGGAAGTTTTCTCG, reverse gRNA gRR88), SEQ ID NO:23 (AAATGGGACTTGAGATATGT, reverse gRNA gRR259), SEQ ID NO:24 (CCCTTGGAGAAGTTTATTAT, forward gRNA gRF481), SEQ ID NO:25 (GTACCCTTACGGTGGTATAA, forward gRNA gRF1265), SEQ ID NO:26 (ATTCCAGCTCGATGAGGGAA, reverse gRNA gRR1291), SEQ ID NO:27 (TACACACAAGCACGTATTTG, forward gRNA gRF1515), and combinations thereof. In some embodiments, the guide RNA sequences are cloned into a vector for introduction to the cell. In some embodiments, the vector also encodes a Cas nuclease (e.g., Cas9 nuclease). In some embodiments, one or more vectors encoding the guide RNA and the Cas nuclease are introduced into the cell in the presence of an HDR oligonucleotide. Cells positive for transformation and the desired CRISPR/Cas mediated editing results may be screened and selected for using standard molecular biology and sequencing techniques known in the art.

In some embodiments, the heterologous nucleic acid of interest encodes a SOLO DANCERS (SDS) and BARNASE fusion gene. The SDS gene encodes a meiosis-specific cyclin and is required for homology interaction during meiotic prophase I in Arabidopsis. The BARNASE gene encodes a ribonuclease, is driven by a tapetum-specific promoter, and, when activated is toxic to and eliminates tapetal cells to create male sterile plants. Co-expression of SDS and BARNASE from a heterologous nucleic acid will create a male and female sterile plant. See, for example, Huang et al. (“Creating completely both male and female sterile plants by specifically ablating microspore and megaspore mother cells,” Frontiers in Plant Science, 2016, 7(30)).

SDS gene from Arabidopsis
(SEQ ID NO: 60)
ATGAAGGAGATCGCGATGAGGAATTCAAAGCGCAAGCCTGAGCCGACGCC
GTTCGCCGGGAAGAAGCTCCGGTCGACGCGATTACGCCGGAAGAGAGCAC
AGATCTCTCCCGTTCTTGTTCAATCACCTCTCTGGAGCAAACAAATCGGA
GTCTCTGCTGCTTCTGTCGATTCCTGCTCCGATTTGCTAGCTGATGACAA
CGTTTCCTGTGGTTCGAGCAGAGTCGAGAAGAGCTCGAATCCGAAGAAGA
CTCTAATTGAAGAGGTAGAAGTTTCTAAACCTGGTTATAATGTGAAGGAG
ACGATTGGTGATTCGAAATTTCGAAGGATTACGAGGTCTTACTCTAAGCT
ACACAAGGAGAAGGAGGGAGATGAGATCGAAGTAAGCGAATCGTCTTGTG
TTGATTCGAATTCTGGTGCTGGATTAAGGAGATTGAATGTGAAGGGAAAT
AAAATTAACGACAACGATGAGATCTCTTTCTCACGATCCGATGTGACCTT
CGCCGGACATGTCTCCAACAGCCGGAGTTTGAATTTCGAATCGGAGAATA
AGGAGAGCGACGTCGTTTCTGTCATATCTGGAGTTGAGTACTGTTCCAAG
TTCGGGAGCGTTACCGGAGGAGCTGATAACGAAGAAATTGAAATCTCCAA
GCCGAGCAGCTTCGTGGAAGCTGATTCCTCTCTTGGATCGGCCAAGGAAT
TGAAGCCGGAGCTTGAGATAGTCGGATGCGTCTCTGATCTCGCTTGCTCT
GAGAAATTCTCGGAAGAGGTTTCGGATTCTCTCGATGATGAGTCATCTGA
GCAACGTTCAGAGATATATTCACAGTATTCCGACTTCGATTACTCGGATT
ACACTCCGTCCATCTTCTTCGACTCTGGCAGCGAATTCTCTGAGAAATCT
TCCTCTGATTCTCCTATTTCACATTCTCGCTCTCTGTACCTCCAGTTCAA
GGAACAGTTCTGTAGATCCACGATTCCCAACGATTTTGGATCTTCTTGCG
AGGAAGAAATTCACTCTGAATTGCTAAGGTTTGATGATGAGGAGGTGGAA
GAGAGCTATCTAAGGCTGAGGGAAAGAGAAAGAAGTCATGCATATATGCG
GGACTGTGCTAAGGCATACTGCTCCAGGATGGACAATACTGGTCTCATCC
CTCGTCTACGCTCCATCATGGTTCAATGGATTGTAAAGCAATGTTCTGAC
ATGGGGCTTCAGCAAGAGACATTGTTTCTAGGAGTTGGTCTGTTGGATCG
ATTCCTGAGCAAAGGATCATTCAAAAGCGAAAGGACTCTAATACTAGTCG
GGATTGCGAGTCTTACTCTGGCCACCAGAATTGAAGAAAATCAACCTTAC
AACAGCATCCGGAAAAGGAACTTCACCATTCAGAACCTAAGATATAGCCG
GCATGAAGTGGTGGCAATGGAGTGGCTGGTTCAAGAAGTCCTCAACTTCA
AATGCTTCACACCCACAATCTTCAACTTCTTGTGGTTCTACTTAAAAGCT
GCTCGAGCCAATCCAGAAGTTGAAAGGAAAGCCAAATCCTTGGCTGTTAC
CTCACTATCCGACCAAACTCAACTCTGTTTTTGGCCCTCAACTGTAGCAG
CTGCACTCGTGGTTCTCGCCTGCATCGAACACAACAAAATCTCTGCATAC
CAACGAGTCATAAAGGTCCATGTTAGAACAACAGATAACGAGTTGCCTGA
ATGCGTTAAGAGTCTGGACTGGTTGCTTGGGCAGTAA
BARNASE gene
(SEQ ID NO: 61)
CTGGAAAACGTCACATTGCTTCCGCATATCGGGTCAGCAACGGCTAAAAT
CCGCTTGAATATGTTCACACAAGCCGCTCAAAACATGATTGACGCCGTAT
ACGGAAGAACGCCGAAAAACCTTACTAAGGAATTTCAATAAGAAGAAAAA
TCCCGGTTGGTTCAGCCGGGGTTTATTTTTCGCTAGATAAAAAGTACTAT
TTTTAAATTCTTTCTATTCCTTTCTTTCGTTGCTGATACAATGAAAAGGA
ATCAGCTTCACATGATGAAAATGGGAGGTATTGCTTTGAAAAAACGATTA
TCGTGGATTTCCGTTTGTTTACTGGTGCTTGTCTCCGCGGCGGGGATGCT
GTTTTCAACAGCTGCCAAAACGGAAACATCTTCTCACAAGGCACACACAG
AAGCACAGGTTATCAACACGTTTGACGGGGTTGCGGATTATCTTCAGACA
TATCATAAGCTACCTGATAATTACATTACAAAATCAGAAGCACAAGCCCT
CGGCTGGGTGGCATCAAAAGGGAACCTTGCAGACGTCGCTCCGGGGAAAA
GCATCGGCGGAGACATCTTCTCAAACAGGGAAGGCAAACTCCCGGGCAAA
AGCGGACGAACATGGCGTGAAGCGGATATTAACTATACATCAGGCTTCAG
AAATTCAGACCGGATTCTTTACTCAAGCGACTGGCTGATTTACAAAACAA
CGGACCATTATCAGACCTTTACAAAAATCAGATAACGAAAAAAACGGCTT
CCCTGCGGAGGCCGTTTTTTTCAGCTTTACATAAAGTGTGTAATAAATTT
TTCTTCAAACTCTGATCGGTCAATTTCACTTT

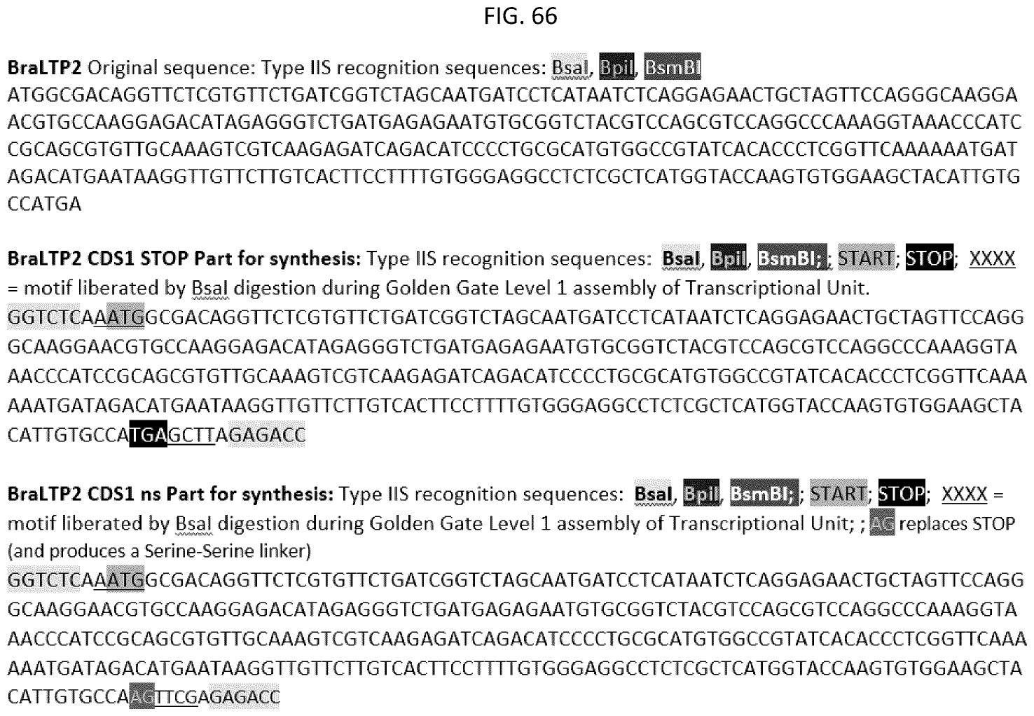

In some embodiments, provided herein is a Cannabis plant with increased trichomes that has increased expression of endogenous Cannabis lipid transfer protein 2 (LTP2) or includes a heterologous nucleic acid encoding Brassica napus LTP2 (BraLTP2). In some embodiments, a Cannabis cell is transformed with a heterologous polynucleotide encoding a polypeptide at least 80%, 85%, 90%, 95%, 98%, 99%, or 99.9% identical to SEQ ID NO:28. In some embodiments, a Cannabis cell is transformed with a heterologous polynucleotide encoding the polypeptide of SEQ ID NO:28. In some embodiments, the heterologous polynucleotide is at least 80%, 85%, 90%, 95%, 98%, 99%, or 99.9% identical to SEQ ID NO:29. The heterologous nucleic acid encoding BraLTP2 may be incorporated into a construct or vector as described herein. One embodiment for cloning BraLTP2 into a vector suitable for transformation using the methods described herein is demonstrated in FIG. 66. Trichomes in the transformed Cannabis plant may be at least 2 fold, 5 fold, 10 fold, or 15 fold higher than trichomes in a Cannabis plant that does not include the heterologous polynucleotide. In some embodiments, the modulation of the LTP2 gene in other plants, such as Brassica napus is described in the art. See, for example, Tian et al. (Tian et al., “Overexpression of BraLTP2, a lipid transfer protein of Brassica napus, results in increased trichome density and altered concentration of secondary metabolites,” Int. J. Mol. Sci., 2018, 19:1733).

BraLTP2 protein sequence
(SEQ ID NO: 28)
MATGSRVLIGLAMILIISGELLVPGQGTCQGDIEGLMRECAVYVQRPGPK
VNPSAACCKVVKRSDIPCACGRITPSVQKMIDMNKVVLVTSFCGRPLAHG
TKCGSYIVP
BraLTP2 gene sequence GenBank: KM062522.1
(SEQ ID NO: 29)
ATGGCGACAGGTTCTCGTGTTCTGATCGGTCTAGCAATGATCCTCATAAT
CTCAGGAGAACTGCTAGTTCCAGGGCAAGGAACGTGCCAAGGAGACATAG
AGGGTCTGATGAGAGAATGTGCGGTCTACGTCCAGCGTCCAGGCCCAAAG
GTAAACCCATCCGCAGCGTGTTGCAAAGTCGTCAAGAGATCAGACATCCC
CTGCGCATGTGGCCGTATCACACCCTCGGTTCAAAAAATGATAGACATGA
ATAAGGTTGTTCTTGTCACTTCCTTTTGTGGGAGGCCTCTCGCTCATGGT
ACCAAGTGTGGAAGCTACATTGTGCCATGA

In some embodiments, the heterologous nucleic acid of interest modulates the expression or function of the endogenous Cannabis LTP2 gene. A heterologous nucleic acid is introduced into the Cannabis explant to upregulate, overexpress, or provide multiple copies of the LPT2 gene. Cannabis plants grown from the transformed Cannabis explant are characterized by a phenotype with an increase in trichomes compared to a wild-type plant as well as increased cannabidiol (CBD) production.

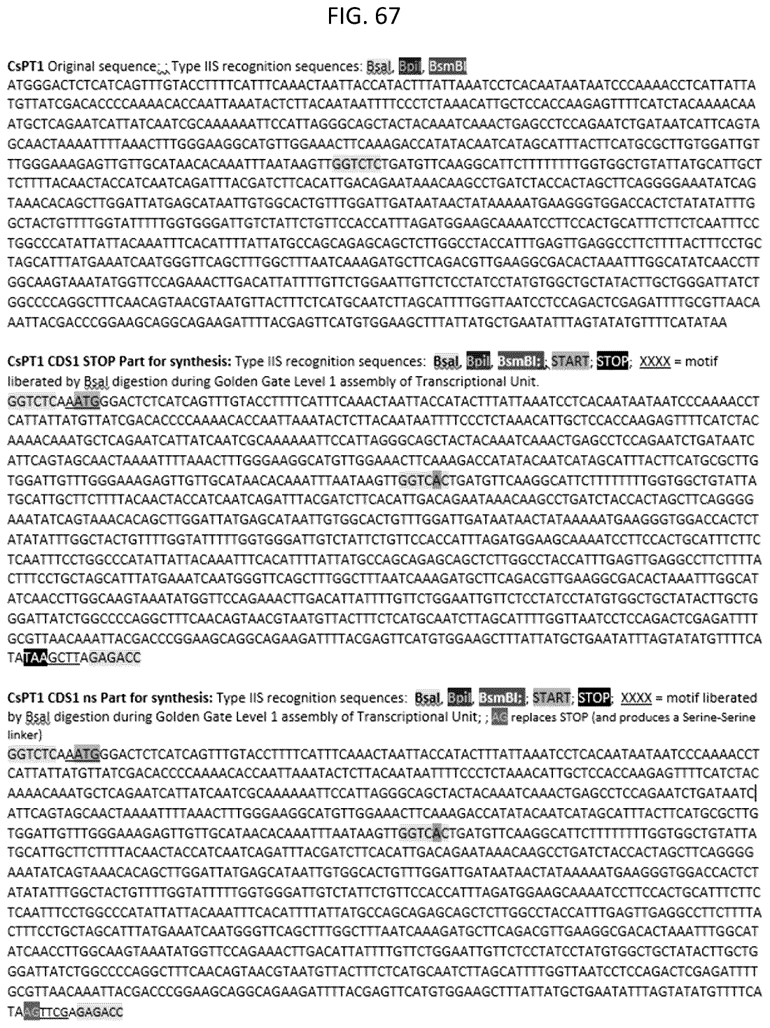

In some embodiments, the heterologous nucleic acid of interest modulates the expression or function of the endogenous Cannabis sativa prenyltransferase 1 (CsPT1) gene. A heterologous nucleic acid is introduced into the Cannabis explant to upregulate, overexpress, or provide multiple copies of the CsPT1 gene. In some embodiments, a Cannabis cell is transformed with a heterologous nucleic acid encoding a polypeptide at least 80%, 85%, 90%, 95%, 98%, 99%, or 99.9% identical to SEQ ID NO:30. In some embodiments, a Cannabis cell is transformed with a heterologous nucleic acid encoding the polypeptide SEQ ID NO:30. In some embodiments, a Cannabis cell is transformed with a heterologous nucleic acid comprising SEQ ID NO:31 or a sequence at least 80%, 85%, 90%, 95%, 98%, 99%, or 99.9% identical thereto. The heterologous nucleic acid encoding CsPT1 may be incorporated into a construct or vector as described herein. One embodiment for cloning CsPT1 into a vector suitable for transformation using the methods described herein is demonstrated in FIG. 67. Cannabis plants grown from the transformed Cannabis explant are characterized by a phenotype with increased cannabigerol (CBG) production and increased cannabidiol (CBD) production. The sequence and activity of the CsPTI gene is known and described in the art. See, for example, Luo et al. (Luo et al., “Complete biosynthesis of cannabinoids and their unnatural analogues in yeast,” Nature, 2019, 567) and U.S. Pat. No. 8,884,100.

CsPT1 protein sequence
(SEQ ID NO: 30)
MGLSSVCTFSFQTNYHTLLNPHNNNPKTSLLCYRHPKTPIKYSYNNFPSK
HCSTKSFHLQNKCSESLSIAKNSIRAATTNQTEPPESDNHSVATKILNFG
KACWKLQRPYTIIAFTSCACGLFGKELLHNTNLISWSLMFKAFFFLVAVL
CIASFTTTINQIYDLHIDRINKPDLPLASGEISVNTAWIMSIIVALFGLI
ITIKMKGGPLYIFGYCFGIFGGIVYSVPPFRWKQNPSTAFLLNFLAHIIT
NFTFYYASRAALGLPFELRPSFTFLLAFMKSMGSALALIKDASDVEGDTK
FGISTLASKYGSRNLTLFCSGIVLLSYVAAILAGIIWPQAFNSNVMLLSH
AILAFWLILQTRDFALTNYDPEAGRRFYEFMWKLYYAEYLVYVFI
CsPT1 cDNA sequence
(SEQ ID NO: 31)
atgggactctcatcagtttgtaccttttcatttcaaactaattaccatac
tttattaaatcctcacaataataatcccaaaacctcattattatgttatc
gacaccccaaaacaccaattaaatactcttacaataattttccctctaaa
cattgctccaccaagagttttcatctacaaaacaaatgctcagaatcatt
atcaatcgcaaaaaattccattagggcagctactacaaatcaaactgagc
ctccagaatctgataatcattcagtagcaactaaaattttaaactttggg
aaggcatgttggaaacttcaaagaccatatacaatcatagcatttacttc
atgcgcttgtggattgtttgggaaagagttgttgcataacacaaatttaa
taagttggtctctgatgttcaaggcattcttttttttggtggctgtatta
gcattgcttcttttacaactaccatcaatcagatttacgatcttcacatt
gacagaataaacaagcctgatctaccactagcttcaggggaaatatcagt
aaacacagcttggattatgagcataattgtggcactgtttggattgataa
taactataaaaatgaagggtggaccactctatatatttggctactgtttt
ggtatttttggtgggattgtctattctgttccaccatttagatggaagca
aaatccttccactgcatttcttctcaatttcctggcccatattattacaa
atttcacattttattatgccagcagagcagctcttggcctaccatttgag
ttgaggccttcttttactttcctgctagcatttatgaaatcaatgggttc
agctttggctttaatcaaagatgcttcagacgttgaaggcgacactaaat
ttggcatatcaaccttggcaagtaaatatggttccagaaacttgacatta
ttttgttctggaattgttctcctatcctatgtggctgctatacttgctgg
gattatctggccccaggctttcaacagtaacgtaatgttactttctcatg
caatcttagcattttggttaatcctccagactcgagattttgcgttaaca
aattacgacccggaagcaggcagaagattttacgagttcatgtggaagct
ttattatgctgaatatttagtatatgttttcatataa

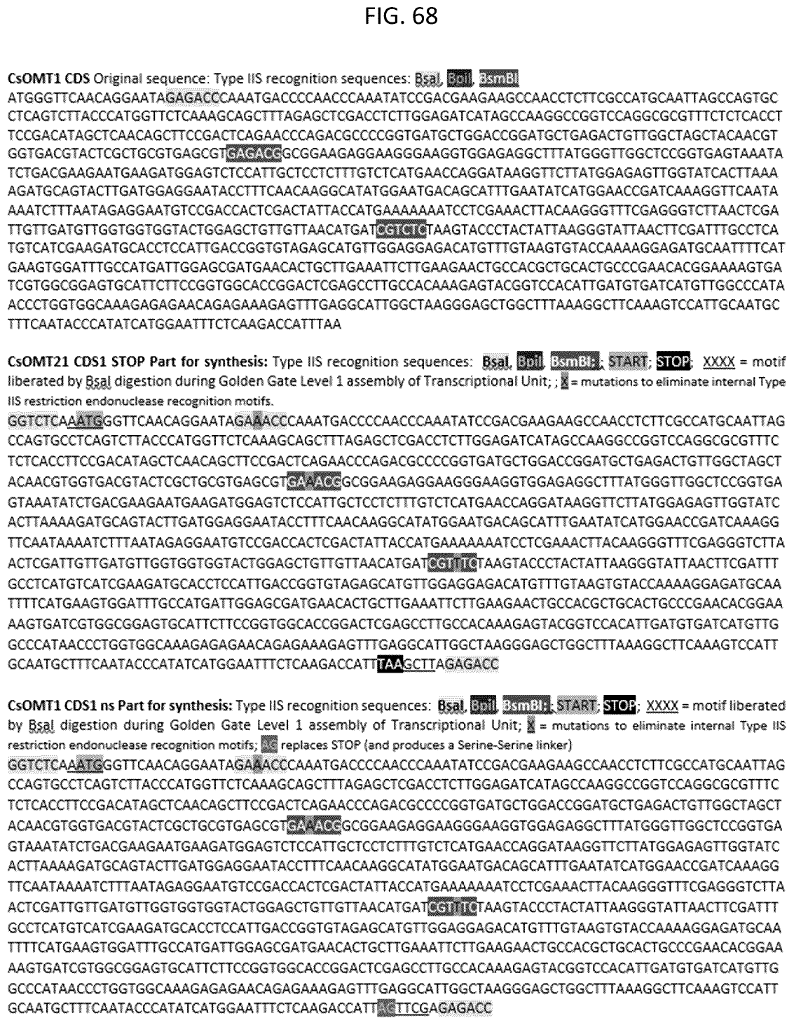

In some embodiments, the heterologous nucleic acid of interest modulates the expression or function of the endogenous Cannabis sativa O-methyltransferase (CsOMT21) gene. A heterologous nucleic acid is introduced into the Cannabis explant to upregulate, overexpress, or provide multiple copies of the CsOMT21 gene. In some embodiments, a Cannabis cell is transformed with a heterologous nucleic acid encoding a polypeptide at least 80%, 85%, 90%, 95%, 98%, 99%, or 99.9% identical to SEQ ID NO:32. In some embodiments, a Cannabis cell is transformed with a heterologous nucleic acid encoding the polypeptide SEQ ID NO:32. In some embodiments, a Cannabis cell is transformed with a heterologous nucleic acid comprising SEQ ID NO:33 or a sequence at least 80%, 85%, 90%, 95%, 98%, 99%, or 99.9% identical thereto. The heterologous nucleic acid encoding CsOMT21 may be incorporated into a construct or vector as described herein. One embodiment for cloning CsOMT21 into a vector suitable for transformation using the methods described herein is demonstrated in FIG. 68. Cannabis plants grown from the transformed Cannabis explant are characterized by a phenotype with increased chrysoeriol production and increased cannflavin A and cannflavin B production. The sequence and activity of the CsOMT21 gene is known and described in the art. See, for example, Rea et al. Phytochemistry 2019: “Biosynthesis of cannflavins A and B from Cannabis sativa L.”

CsOMT21 (PK24150)
(SEQ ID NO: 32)
MGSTGIETQMTPTQISDEEANLFAMQLASASVLPMVLKAALELDLLEIIA
KAGPGAFLSPSDIAQQLPTQNPDAPVMLDRMLRLLASYNVVTYSLRERET
AEEEGKVERLYGLAPVSKYLTKNEDGVSIAPLCLMNQDKVLMESWYHLKD
AVLDGGIPFNKAYGMTAFEYHGTDQRFNKIFNRGMSDHSTITMKKILETY
KGFEGLNSIVDVGGGTGAVVNMIVSKYPTIKGINFDLPHVIEDAPPLTGV
EHVGGDMFVSVPKGDAIFMKWICHDWSDEHCLKFLKNCHAALPEHGKVIV
AECILPVAPDSSLATKSTVHIDVIMLAHNPGGKERTEKEFEALAKGAGFK
GFKVHCNAFNTHIMEFLKTI
Cannabis sativa PK24150.1_1.CasaPuKu, GenBank:
JP459899.1
(SEQ ID NO: 33)
ATGGGTTCAACAGGAATAGAGACCCAAATGACCCCAACCCAAATATCCGA
CGAAGAAGCCAACCTCTTCGCCATGCAATTAGCCAGTGCCTCAGTCTTAC
CCATGGTTCTCAAAGCAGCTTTAGAGCTCGACCTCTTGGAGATCATAGCC
AAGGCCGGTCCAGGCGCGTTTCTCTCACCTTCCGACATAGCTCAACAGCT
TCCGACTCAGAACCCAGACGCCCCGGTGATGCTGGACCGGATGCTGAGAC
TGTTGGCTAGCTACAACGTGGTGACGTACTCGCTGCGTGAGCGTGAGACG
GCGGAAGAGGAAGGGAAGGTGGAGAGGCTTTATGGGTTGGCTCCGGTGAG
TAAATATCTGACGAAGAATGAAGATGGAGTCTCCATTGCTCCTCTTTGTC
TCATGAACCAGGATAAGGTTCTTATGGAGAGTTGGTATCACTTAAAAGAT
GCAGTACTTGATGGAGGAATACCTTTCAACAAGGCATATGGAATGACAGC
ATTTGAATATCATGGAACCGATCAAAGGTTCAATAAAATCTTTAATAGAG
GAATGTCCGACCACTCGACTATTACCATGAAAAAAATCCTCGAAACTTAC
AAGGGTTTCGAGGGTCTTAACTCGATTGTTGATGTTGGTGGTGGTACTGG
AGCTGTTGTTAACATGATCGTCTCTAAGTACCCTACTATTAAGGGTATTA
ACTTCGATTTGCCTCATGTCATCGAAGATGCACCTCCATTGACCGGTGTA
GAGCATGTTGGAGGAGACATGTTTGTAAGTGTACCAAAAGGAGATGCAAT
TTTCATGAAGTGGATTTGCCATGATTGGAGCGATGAACACTGCTTGAAAT
TCTTGAAGAACTGCCACGCTGCACTGCCCGAACACGGAAAAGTGATCGTG
GCGGAGTGCATTCTTCCGGTGGCACCGGACTCGAGCCTTGCCACAAAGAG
TACGGTCCACATTGATGTGATCATGTTGGCCCATAACCCTGGTGGCAAAG
AGAGAACAGAGAAAGAGTTTGAGGCATTGGCTAAGGGAGCTGGCTTTAAA
GGCTTCAAAGTCCATTGCAATGCTTTCAATACCCATATCATGGAATTTCT
CAAGACCATTTAA

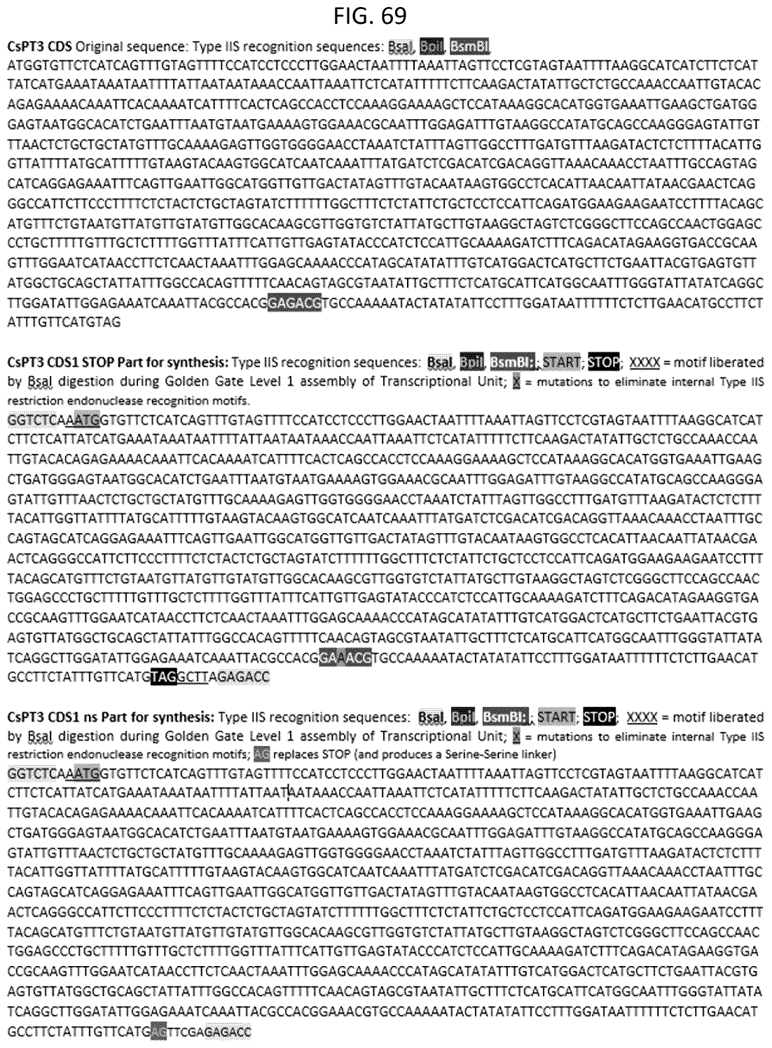

In some embodiments, the heterologous nucleic acid of interest modulates the expression or function of the endogenous Cannabis sativa prenyltransferace 3 (CsPT3) gene. A heterologous nucleic acid is introduced into the Cannabis explant to upregulate, overexpress, or provide multiple copies of the CsPT3 gene. In some embodiments, a Cannabis cell is transformed with a heterologous nucleic acid encoding a polypeptide at least 80%, 85%, 90%, 95%, 98%, 99%, or 99.9% identical to SEQ ID NO:34. In some embodiments, a Cannabis cell is transformed with a heterologous nucleic acid encoding the polypeptide SEQ ID NO:34. In some embodiments, a Cannabis cell is transformed with a heterologous nucleic acid comprising SEQ ID NO:35 or a sequence at least 80%, 85%, 90%, 95%, 98%, 99%, or 99.9% identical thereto. The heterologous nucleic acid encoding CsPT3 may be incorporated into a construct or vector as described herein. One embodiment for cloning CsPT3 into a vector suitable for transformation using the methods described herein is demonstrated in FIG. 69. Cannabis plants grown from the transformed Cannabis explant are characterized by a phenotype with increased cannabigerol (CBG) production and increased cannabidiol (CBD) production.

CsPT3 (PK17697)
(SEQ ID NO: 34)
MVFSSVCSFPSSLGTNFKLVPRSNFKASSSHYHEINNFINNKPIKFSYFS
SRLYCSAKPIVHRENKFTKSFSLSHLQRKSSIKAHGEIEADGSNGTSEFN
VMKSGNAIWRFVRPYAAKGVLFNSAAMFAKELVGNLNLFSWPLMFKILSF
TLVILCIFVSTSGINQIYDLDIDRINKPNLPVASGEISVELAWLLTIVCT
ISGLTLTIITNSGPFFPFLYSASIFFGFLYSAPPFRWKKNPFTACFCNVM
LYVGTSVGVYYACKASLGLPANWSPAFCLLFWFISLLSIPISIAKDLSDI
EGDRKFGIITFSTKFGAKPIAYICHGLMLLNYVSVMAAAIIWPQFFNSSV
ILLSHAFMAIWVLYQAWILEKSNYATETCQKYYIFLWIIFSLEHAFYLFM
Cannabis sativa PK17697.1 1.CasaPuKu,
GenBank: JP460361.1,
(SEQ ID NO: 35)
atggtgttctcatcagtttgtagttttccatcctcccttggaactaattt
taaattagttcctcgtagtaattttaaggcatcatcttctcattatcatg
aaataaataattttattaataataaaccaattaaattctcatatttttct
tcaagactatattgctctgccaaaccaattgtacacagagaaaacaaatt
cacaaaatcattttcactcagccacctccaaaggaaaagctccataaagg
cacatggtgaaattgaagctgatgggagtaatggcacatctgaatttaat
gtaatgaaaagtggaaacgcaatttggagatttgtaaggccatatgcagc
caagggagtattgtttaactctgctgctatgtttgcaaaagagttggtgg
ggaacctaaatctatttagttggcctttgatgtttaagatactctctttt
acattggttattttatgcatttttgtaagtacaagtggcatcaatcaaat
ttatgatctcgacatcgacaggttaaacaaacctaatttgccagtagcat
caggagaaatttcagttgaattggcatggttgttgactatagtttgtaca
ataagtggcctcacattaacaattataacgaactcagggccattcttccc
ttttctctactctgctagtatcttttttggctttctctattctgctcctc
cattcagatggaagaagaatccttttacagcatgtttctgtaatgttatg
ttgtatgttggcacaagcgttggtgtctattatgcttgtaaggctagtct
cgggcttccagccaactggagccctgctttttgtttgctcttttggttta
tttcattgttgagtatacccatctccattgcaaaagatctttcagacata
gaaggtgaccgcaagtttggaatcataaccttctcaactaaatttggagc
aaaacccatagcatatatttgtcatggactcatgcttctgaattacgtga
gtgttatggctgcagctattatttggccacagtttttcaacagtagcgta
atattgctttctcatgcattcatggcaatttgggtattatatcaggcttg
gatattggagaaatcaaattacgccacggagacgtgccaaaaatactata
tattcctttggataattttttctcttgaacatgccttctatttgttcatg
tag

In some embodiments, the heterologous nucleic acid of interest produces a glyphosate resistant Cannabis plant following transformation. To create a glyphosate resistant Cannabis plant, a Cannabis cell is transformed as described herein with a heterologous nucleic suitable for mutating the Cannabis sativa gene encoding 3-phosphoshikimate 1-carboxyvinyltransferase 2 (EPSP synthase) such that when expressed, the EPSP synthase enzyme is not inhibited by the herbicide glyphosate. Without wishing to be bound by any particular theory or embodiment, glyphosate is a competitive inhibitor of phosphoenolpyruvate (PEP) in EPSP, acting as a transition state analog that binds more tightly to the EPSPS-S3P (shikimate-3-phosphate bound EPSP synthase) complex than PEP. Upon exposure to glyphosate, the EPSP synthase enzyme is non-functional or severely inhibited resulting in plant death. However, it is possible to mutate the EPSP synthase enzyme such at it does not bind glyphosate but still catalyzes the synthesis of 5-enol-pyruvylshikimate-3-phosphate.

In some embodiments, the Cannabis sativa gene encoding EPSP synthase is mutated using prime editing guide RNA (pegRNA) together with a Cas9 nuclease and reverse transcriptase fusion protein. In some embodiments, the Cas9 nuclease is a mutated Cas9 nuclease that only cleaves a single strand of the target DNA. In some embodiments, the mutated Cas9 nuclease is a Cas9 H840A nickase or a Cas9 D10A nickase (Cas9n). pegRNA are designed with the desired genetic mutation and to target the Cannabis sativa EPSPS gene loci of interest. A nucleic acid encoding the pegRNA and the Cas9 nuclease/reverse transcriptase fusion protein is introduced into a Cannabis sativa cell. Cells positive for transformation and the desired prime editing mutations may be screened and selected for using standard molecular biology and sequencing techniques known in the art.

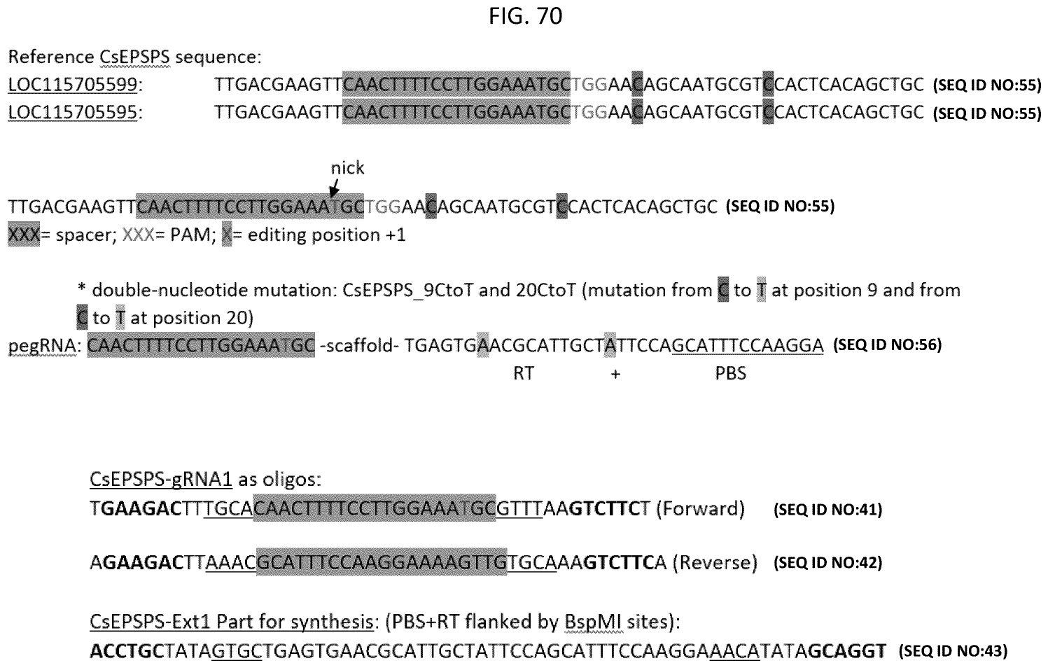

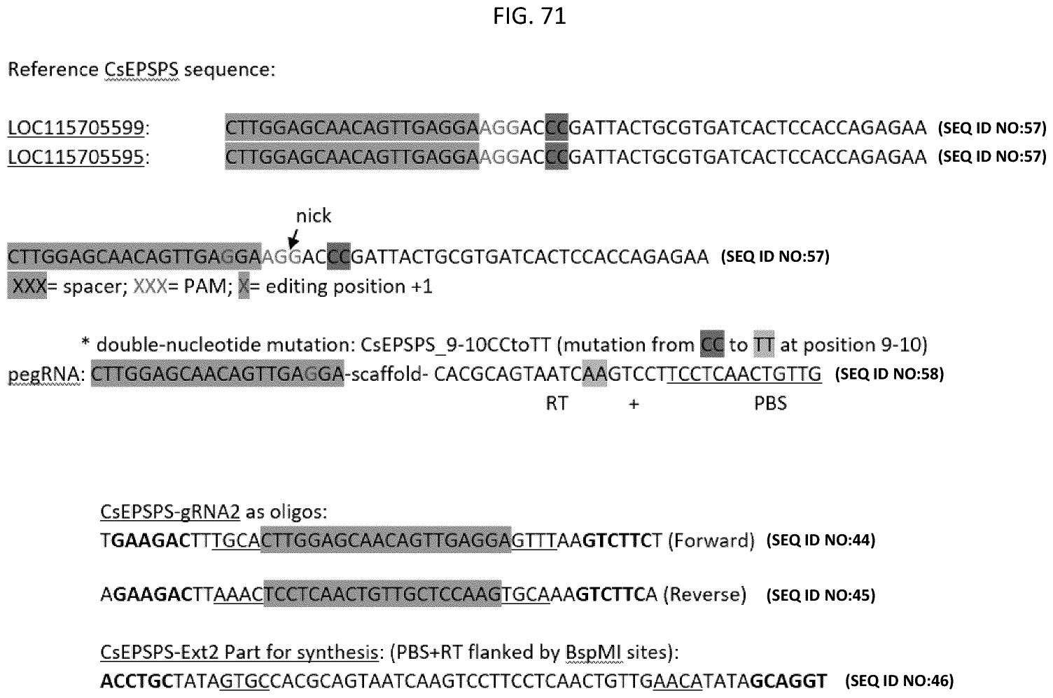

In some embodiments, the Cannabis EPSPS gene (SEQ ID NO:40) is mutated at positions 1790, 1801, 3620, and 3621 to encode an EPSP synthase that is glyphosate resistant and includes T181I, P185S, and P460L mutations relative to the wild-type sequence. In some embodiments, the pegRNA for the mutations at positions 1790 and 1801 comprises the gRNA sequences tgaagactttgcaCAACTTTTCCTTGGAAATGCgtttaagtettct (forward, SEQ ID NO:41) and AGAAGACTTAAACGCATTTCCAAGGAAAAGTTGTGCAAAGTCTTCA (reverse, SEQ ID NO:42) and the sequence ACCTGCTATAGTGCTGAGTGAACGCATTGCTATTCCAGCATTTCCAAGGAAACATAT AGCAGGT (SEQ ID NO:43) encoding the two C→T mutations at positions 1790 and 1801. See, FIG. 70. In some embodiments, the pegRNA for the mutations at positions 3620 and 3621comprises the gRNA sequences tgaagactttgcaCTTGGAGCAACAGTTGAGGAgtttaagtcttct (forward, SEQ ID NO:44) and AGAAGACTTAAACTCCTCAACTGTTGCTCCAAGTGCAAAGTCTTCA (reverse, SEQ ID NO:45) and the sequence ACCTGCTATAGTGCCACGCAGTAATCAAGTCCTTCCTCAACTGTTGAACATATAGCA GGT (SEQ ID NO:46) encoding the two C→T mutations at positions 3620 and 3621. See, FIG. 71.

Cannabis EPSP synthase
(SEQ ID NO: 36)
MAQVSKICSNGAQTILTLPNISKSHTPRSLNSVSLRSPFLGSSNSLSLKI
GTEFGGCSTVGKAMAGPVMASAVTAEKPSKVPEIVLQPIKDISGTVKLPG
SKSLSNRILLLAALSEGTTVVDNLLDSDDIHYMLGALETLGLRVEADKES
KRAIVEGCAGQFPAGKESVDEVQLFLGNAGTAMRPLTAAVTVAGGNASYV
LDGVPRMRERPIGDLVTGLKQLGADVDCFHGTDCPPVRVLGKGGLPGGKV
KLSGSISSQYLTALLMAAPLALGDVEIEIIDKLISVPYVDMTLKLMARFG
VTVEHSDSWDRFLVKGGQKYKSPGNAYVEGDASSASYFLAGAAVTGGTVT
VEGCGTSSLQGDVKFAEVLEKMGAKVSWTENSVTVTGPPRDSVKSKHLKA
IDVNMNKMPDVAMTLAVVALFADGPTAIRDVASWRVKETERMIAICTELR
KLGATVEEGPDYCVITPPEKLNITAIDTYDDHRMAMAFSLAACSDVPVTI
KDPGCTRKTFPDYFEVLERFTKH
Glyphosate resistant Cannabis EPSP synthase (SEQ
ID NO: 37), mutations relative to SEQ ID NO: 36
shown in bold underline including T181I, P185S,
P460L
MAQVSKICSNGAQTILTLPNISKSHTPRSLNSVSLRSPFLGSSNSLSLKI
GTEFGGCSTVGKAMAGPVMASAVTAEKPSKVPEIVLQPIKDISGTVKLPG
SKSLSNRILLLAALSEGTTVVDNLLDSDDIHYMLGALETLGLRVEADKES
KRAIVEGCAGQFPAGKESVDEVQLFLGNAGIAMRSLTAAVTVAGGNASYV
LDGVPRMRERPIGDLVTGLKQLGADVDCFHGTDCPPVRVLGKGGLPGGKV
KLSGSISSQYLTALLMAAPLALGDVEIEIIDKLISVPYVDMTLKLMARFG
VTVEHSDSWDRFLVKGGQKYKSPGNAYVEGDASSASYFLAGAAVTGGTVT
VEGCGTSSLQGDVKFAEVLEKMGAKVSWTENSVTVTGPPRDSVKSKHLKA
IDVNMNKMPDVAMTLAVVALFADGPTAIRDVASWRVKETERMIAICTELR
KLGATVEEGLDYCVITPPEKLNITAIDTYDDHRMAMAFSLAACSDVPVTI
KDPGCTRKTFPDYFEVLERFTKH
Cannabis EPSP synthase cDNA
(SEQ ID NO: 38)
ATGGCCCAAGTGAGCAAAATCTGTAGCAATGGAGCTCAAACTATCCTTAC
TCTCCCAAATATATCTAAGTCTCATACACCAAGATCCCTAAATTCAGTTT
CGTTGAGATCACCGTTTTTGGGTTCATCTAACTCTTTGAGTTTGAAGATT
GGAACTGAATTTGGGGGTTGTTCTACGGTTGGTAAAGCTATGGCTGGTCC
AGTCATGGCTTCAGCTGTCACAGCGGAGAAGCCTTCAAAGGTACCGGAGA
TTGTGTTGCAGCCCATTAAAGATATCTCTGGCACTGTCAAGTTGCCGGGT
TCCAAGTCACTATCGAATCGGATTCTACTCCTGGCTGCTCTTTCTGAGGG
GACAACTGTTGTGGACAACTTGTTAGATAGTGATGACATTCACTACATGC
TTGGTGCCTTGGAAACCCTTGGTCTTCGTGTTGAAGCAGACAAGGAAAGC
AAACGAGCAATTGTGGAAGGTTGTGCGGGTCAGTTTCCTGCAGGTAAAGA
ATCTGTTGACGAAGTTCAACTTTTCCTTGGAAATGCTGGAACAGCAATGC
GTCCACTCACAGCTGCGGTGACTGTTGCTGGTGGAAATGCTAGCTACGTA
CTTGATGGTGTTCCTCGAATGAGAGAAAGACCAATTGGAGATTTGGTGAC
TGGTCTTAAGCAGCTTGGTGCAGATGTTGATTGTTTTCATGGTACGGATT
GTCCCCCTGTTCGTGTGCTTGGAAAAGGAGGCCTTCCTGGGGGCAAGGTG
AAACTTTCTGGATCAATTAGCAGTCAATATTTGACAGCCTTGCTTATGGC
AGCTCCCTTGGCTCTTGGAGATGTTGAAATCGAGATAATTGATAAATTGA
TCTCGGTTCCCTATGTTGATATGACTTTGAAGTTGATGGCACGTTTTGGG
GTTACTGTTGAACACAGTGATAGCTGGGATCGATTTTTAGTTAAAGGAGG
TCAAAAGTACAAATCTCCTGGAAACGCTTATGTTGAAGGTGATGCTTCAA
GTGCTAGTTACTTCCTAGCTGGTGCTGCAGTCACTGGTGGTACAGTCACC
GTAGAAGGTTGTGGGACTAGTAGTTTACAGGGAGACGTAAAATTTGCTGA
AGTTCTTGAGAAAATGGGTGCTAAAGTTAGCTGGACAGAGAACAGTGTCA
CGGTCACTGGACCACCACGAGATTCTGTAAAAAGTAAACACTTGAAAGCC
ATTGATGTCAACATGAACAAAATGCCTGATGTTGCCATGACTCTTGCTGT
AGTTGCTCTTTTTGCTGATGGCCCCACTGCTATAAGAGATGTGGCAAGTT
GGAGAGTCAAGGAGACAGAGAGAATGATTGCCATCTGCACTGAACTCAGA
AAGCTTGGAGCAACAGTTGAGGAAGGACCCGATTACTGCGTGATCACTCC
ACCAGAGAAACTAAATATCACAGCAATAGACACATACGACGACCACAGGA
TGGCTATGGCGTTCTCTCTTGCAGCTTGTTCAGATGTGCCAGTTACCATT
AAGGATCCTGGTTGCACCCGAAAAACTTTCCCAGATTACTTTGAAGTCCT
TGAGAGATTTACAAAGCACTGA

Glyphosate resistant Cannabis EPSP synthase cDNA (SEQ ID NO:39), mutations relative to SEQ ID NO:38 shown in bold underline, including C542T, C553T, C1379T, and C1380T

ATGGCCCAAGTGAGCAAAATCTGTAGCAATGGAGCTCAAACTATCCTTA
CTCTCCCAAATATATCTAAGTCTCATACACCAAGATCCCTAAATTCAGT
TTCGTTGAGATCACCGTTTTTGGGTTCATCTAACTCTTTGAGTTTGAAG
ATTGGAACTGAATTTGGGGGTTGTTCTACGGTTGGTAAAGCTATGGCTG
GTCCAGTCATGGCTTCAGCTGTCACAGCGGAGAAGCCTTCAAAGGTACC
GGAGATTGTGTTGCAGCCCATTAAAGATATCTCTGGCACTGTCAAGTTG
CCGGGTTCCAAGTCACTATCGAATCGGATTCTACTCCTGGCTGCTCTTT
CTGAGGGGACAACTGTTGTGGACAACTTGTTAGATAGTGATGACATTCA
CTACATGCTTGGTGCCTTGGAAACCCTTGGTCTTCGTGTTGAAGCAGAC
AAGGAAAGCAAACGAGCAATTGTGGAAGGTTGTGCGGGTCAGTTTCCTG
CAGGTAAAGAATCTGTTGACGAAGTTCAACTTTTCCTTGGAAATGCTGG
AATAGCAATGCGTTCACTCACAGCTGCGGTGACTGTTGCTGGTGGAAAT
GCTAGCTACGTACTTGATGGTGTTCCTCGAATGAGAGAAAGACCAATTG
GAGATTTGGTGACTGGTCTTAAGCAGCTTGGTGCAGATGTTGATTGTTT
TCATGGTACGGATTGTCCCCCTGTTCGTGTGCTTGGAAAAGGAGGCCTT
CCTGGGGGCAAGGTGAAACTTTCTGGATCAATTAGCAGTCAATATTTGA
CAGCCTTGCTTATGGCAGCTCCCTTGGCTCTTGGAGATGTTGAAATCGA
GATAATTGATAAATTGATCTCGGTTCCCTATGTTGATATGACTTTGAAG
TTGATGGCACGTTTTGGGGTTACTGTTGAACACAGTGATAGCTGGGATC
GATTTTTAGTTAAAGGAGGTCAAAAGTACAAATCTCCTGGAAACGCTTA
TGTTGAAGGTGATGCTTCAAGTGCTAGTTACTTCCTAGCTGGTGCTGCA
GTCACTGGTGGTACAGTCACCGTAGAAGGTTGTGGGACTAGTAGTTTAC
AGGGAGACGTAAAATTTGCTGAAGTTCTTGAGAAAATGGGTGCTAAAGT
TAGCTGGACAGAGAACAGTGTCACGGTCACTGGACCACCACGAGATTCT
GTAAAAAGTAAACACTTGAAAGCCATTGATGTCAACATGAACAAAATGC
CTGATGTTGCCATGACTCTTGCTGTAGTTGCTCTTTTTGCTGATGGCCC
CACTGCTATAAGAGATGTGGCAAGTTGGAGAGTCAAGGAGACAGAGAGA
ATGATTGCCATCTGCACTGAACTCAGAAAGCTTGGAGCAACAGTTGAGG
AAGGACTTGATTACTGCGTGATCACTCCACCAGAGAAACTAAATATCAC
AGCAATAGACACATACGACGACCACAGGATGGCTATGGCGTTCTCTCTT
GCAGCTTGTTCAGATGTGCCAGTTACCATTAAGGATCCTGGTTGCACCC
GAAAAACTTTCCCAGATTACTTTGAAGTCCTTGAGAGATTTACAAAGCA
CTGA

Cannabis EPSP synthase gene, Cannabis sativa chromosome 2, cs10, whole genome shotgun sequence, Cannabis sativa 3-phosphoshikimate 1-carboxyvinyltransferase 2 (LOC115705599), (SEQ ID NO:40), possible glyphosate resistant mutation target loci indicated in bold underline italics including positions 1790, 1801, 3620, and 3621.

GGTTGGTAAGCCCTCCTACCCTCTTTGAAAATTGAAAGAGAGTCAATGTC
GACCTACAGCAGCAGCATCCATTAACGTTACCATTGCCACCAAAAATCCA
ACCTTTATTTGTATAGAGAGAATCAGAGAAGGTTTGGGTTTCAGAGAGAG
AGAGAAGAAGAACAAAAAAATGGCCCAAGTGAGCAAAATCTGTAGCAATG
GAGCTCAAACTATCCTTACTCTCCCAAATATATCTAAGTCTCATACACCA
AGATCCCTAAATTCAGTTTCGTTGAGATCACCGTTTTTGGGTTCATCTAA
CTCTTTGAGTTTGAAGATTGGAACTGAATTTGGGGGTTGTTCTACGGTTG
GTAAAGCTATGGCTGGTCCAGTCATGGCTTCAGCTGTCACAGCGGAGAAG
CCTTCAAAGGTACCGGAGATTGTGTTGCAGCCCATTAAAGATATCTCTGG
CACTGTCAAGTTGCCGGGTTCCAAGTCACTATCGAATCGGATTCTACTCC
TGGCTGCTCTTTCTGAGGTATATTTCATTTTTTTTAAAACGTCAAACATG
TATTTTTGTCGAGGAAGTTTTCTGTATATACAAAGATAAGAGAGTAAAAA
TATGGAACATCAATACCAAAATGAACCAAAACTAGGCTAAGCTATCAAAT
CATGTCATGGTATGCCATACTCTACTTTCCTATCTCAAGCTCCACAGCTA
TAAAATACTATATCGTAATTATTTTGTCAACTGCTTTCATATTCCTTGTA
ATTTCCCTCATTCCCACTAAAACTAGTTCCAATGGATTGTGTGGCTGGAA
ACTGTAGTTAGTTACATTAGCTAGATCTGAACCATGATCAGCATCGACTG
CCCAACTGGTAAACCATGTAATTGCATGGAATTCTTCCTTTGTTATCCAC
AAATTTGAAAAGTATTTTTGAGGTATACAAAGATTGTGCTTTTTATGAGC
AATTTTCTTTTAGTTTTATGTTAAGAGTTTGTAGCGATGGGATGTTTTTT
TTCTAGAAAATGGACAGTAAAGCTTAGCATTTTTACTTTATTGGTGTAAA
TGAATAGTGTTCATTGAAGCTGAACTCATGCCCTTAATTGGGAGGAAAAT
TGAGAGAAATGGAGTAAAGTAATATGATATTTTGGTTAAATTCGTAAGAA
TATGATGGAAATAAAAAATGCAACTCAACTGGGTTACTGAAGTTATATTT
CTGGTCTCAGTTGTGCTTTTACAACTTTAGTCTAGAGCTCCACGCTGCGG
AGAGATTCGGAGTCCTTACAGTTTATTTTGATAATGATTTATGAGAATTT
CATAACTCTACGCTTTTGTTACATTATATATGAGGTGTTTCGTTGGTGCA
TTGTCTCACCTGAACTCCCTAAATTTTAGAATGTGGGATTTAGAATGAAG
TTATACTATTAGTGTTTGAGTCATCTAGAATTTGTAGCTGCTCATTCTCC
ATATACTCTTTCTCTATTTCCTCCCCATATTTTGGCGCTACTACTTATCT
TTACAGTTCATGTTATTTTCATGTACTTGAGTTTTTTGCCCTATAAAATA
TTTTGAGCGGTGGGAAGTAACTGTTTTTTTTGTTATAATTATCCAGGGGA
CAACTGTTGTGGACAACTTGTTAGATAGTGATGACATTCACTACATGCTT
GGTGCCTTGGAAACCCTTGGTCTTCGTGTTGAAGCAGACAAGGAAAGCAA
ACGAGCAATTGTGGAAGGTTGTGCGGGTCAGTTTCCTGCAGGTAAAGAAT
CTGTTGACGAAGTTCAACTTTTCCTTGGAAATGCTGGAACCAGCAATGCG
TCCACTCACAGCTGCGGTGACTGTTGCTGGTGGAAATGCTAGGTTTGTCT
TCATTGCAATTGCTTTTGAATATAAAGTACTTCTAATGCAGTGAATTTAT
GCTCTTGTTTTTCTTACTGGCCGAGTAGCTCTTACATTTTAGGTAAAGAA
AGTCACTTTTGCTAACAACATCACCATTTATACTTCCCTCTTTACTTTGA
TGTGGTTATGCTAGAAATTACATGTTGGAAATGAACTAGCACATATCATA
AATTATTTTGTATGCTGTTATTACATTTTCTCAGTAACCTCTTAACTTCT
ATATCTCAGCTACGTACTTGATGGTGTTCCTCGAATGAGAGAAAGACCAA
TTGGAGATTTGGTGACTGGTCTTAAGCAGCTTGGTGCAGATGTTGATTGT
TTTCATGGTACGGATTGTCCCCCTGTTCGTGTGCTTGGAAAAGGAGGCCT
TCCTGGGGGCAAGGTGAGGCTTGCATTGCTTCTTCTTATTCTTTTTGGCC
ATAAAACATCATTGTAATAGTGGTTTTATGTTATGAAATCCATTGACTGG
TTTATTTTTAGGTTGTTGTTTTGCTTTTAAATAAAAACAATATTGTCAAA
TGATGCATAAGTAGTGATTACATCTACATCATTTAATTTATTATCTTAAA
TGATGACAAACTTCATCATTTTGACTCAGAATTATGTAATATTACCCTTT
GCAGGTGAAACTTTCTGGATCAATTAGCAGTCAATATTTGACAGCCTTGC
TTATGGCAGCTCCCTTGGCTCTTGGAGATGTTGAAATCGAGATAATTGAT
AAATTGATCTCGGTTCCCTATGTTGATATGACTTTGAAGTTGATGGCACG
TTTTGGGGTTACTGTTGAACACAGTGATAGCTGGGATCGATTTTTAGTTA
AAGGAGGTCAAAAGTACAAGTAGGTTTCTTCTGAATATAGTTGATAGTAT
TGTTACATTACATCTGGTTATGTCAAAGAGTAATAAATTGAAAAATAAAA
ATCTGTCAGATCTCCTGGAAACGCTTATGTTGAAGGTGATGCTTCAAGTG
CTAGTTACTTCCTAGCTGGTGCTGCAGTCACTGGTGGTACAGTCACCGTA
GAAGGTTGTGGGACTAGTAGTTTACAGGTATTTTGCTTAGACCTTGAAAT
CTCTTATTCTTGTACTTGTGTTTACATAGAATCTAAGATTAAGTGTATTT
ACATACATTAACTGGTGTTTAATAAAGGGAGACGTAAAATTTGCTGAAGT
TCTTGAGAAAATGGGTGCTAAAGTTAGCTGGACAGAGAACAGTGTCACGG
TCACTGGACCACCACGAGATTCTGTAAAAAGTAAACACTTGAAAGCCATT
GATGTCAACATGAACAAAATGCCTGATGTTGCCATGACTCTTGCTGTAGT
TGCTCTTTTTGCTGATGGCCCCACTGCTATAAGAGATGGTATGTTTTTCC
TTAAATTTGTGAGATGGTAAAATGGGGCAGTCGGTTTGGGTTGGGGTAGA
TTATCGGTTCTCGTCTGCCATAATAAAAAATAATCTGCTCATTTGCAATA
AATTTCACAGACACAAAAATGAAAACCAATAAAATATTATTTTGTTTAGA
GGATTAAATACTCATTTCTTGCCTTTCCTAATTCCCAGTGGCAAGTTGGA
GAGTCAAGGAGACAGAGAGAATGATTGCCATCTGCACTGAACTCAGAAAG
GTTAGTTTTTATGCTGTTTTATGTACTTGTTATGTCATGCGCCTTGGAAT
GTAATGGCTGATAGCTATCTGTTCTTATGGGAACAAACATTTCAGCTTGG
AGCAACAGTTGAGGAAGGACCCGATTACTGCGTGATCACTCCACCAGAGA
AACTAAATATCACAGCAATAGACACATACGACGACCACAGGATGGCTATG
GCGTTCTCTCTTGCAGCTTGTTCAGATGTGCCAGTTACCATTAAGGATCC
TGGTTGCACCCGAAAAACTTTCCCAGATTACTTTGAAGTCCTTGAGAGAT
TTACAAAGCACTGAATGAGTATTTATTAACTGGATAGAGAACAATAGCAT
CGGCTACTGTCATTACAACTAAAGCAGTTGGGAGGCAGGCAATCCTTTTC
AATTATCATGTGTTTGATTTTGGTCGACTGTATTGCAAGTTGAGCTTCTC
ATTATTATTAAGACTGTAATCGTAGTTATTTGTTGTAACTTCTGCAAACC
CTTCATGTTATTTTTTCACCCTTCTAATAAGCCAGTGGGGCAAATTCTAT
ATCTGCTATATGAGCTTGAGTGTAGAGAGAACTTTTGTCAATGTATAGGT
TTCTAGCAGAAGCACCATCCCTAATATGCTTTATTATAAGAGTTGCTGTG
ATCGTGTAGTGTTATTTTATTGAAAGTAACCGACGCATCTCATATTA

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA or genomic DNA.

As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome-editing technologies. Constructs may also include recombinant polynucleotides created using, for example, recombinant DNA methodologies.

The constructs provided herein may be prepared by methods available to those of skill in the art. Notably each of the constructs used or claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a promoter operably linked to any one of the polynucleotides described herein. The promoter may be a heterologous promoter or an endogenous promoter associated with the heterologous gene, nucleic acid, or polypeptide of interest.

As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the heterologous polynucleotides of interest are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may effect transcription of polynucleotides. In various embodiments, the polynucleotides may be operably linked to at least 1, at least 2, at least 3, at least 4, at least 5, or at least 10 promoters.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. Suitable promoters for expression in plants include, without limitation, the 35S promoter of the cauliflower mosaic virus, ubiquitin, tCUP cryptic constitutive promoter, actin, the Rsyn7 promoter, pathogen-inducible promoters, the maize In2-2 promoter, the tobacco PR-la promoter, glucocorticoid-inducible promoters, estrogen-inducible promoters and tetracycline-inducible and tetracycline-repressible promoters. Other promoters include the T3, T7 and SP6 promoter sequences, which are often used for in vitro transcription of RNA. Those of skill in the art are familiar with a wide variety of additional promoters for use in various cell types. In some embodiments, the heterologous promoter includes a plant promoter, either endogenous to the plant host or heterologous.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Plant mini-chromosomes are also included as vectors. Any suitable vector design known in the art may be used with the explants of the present invention.

Vectors may carry genetic elements, such as those that confer resistance to certain drugs or chemicals. In some embodiments, the vector will additionally include one or more selectable or screenable markers. The selectable or screenable marker may confer upon the plant tissue resistance to an otherwise toxic compound. A number of screenable or selectable marker are known in the art and can be used in the present invention. The screenable marker may be fluorescent (e.g., RFP) or non-fluorescent (e.g., GUS). More than 20 selectable marker genes have been reported in the transformation of higher plants (Komari T, Takakura Y, Ueki J, Kato N, Ishida Y, Hiei Y (2006) Binary vectors and super-binary vectors. In: KanWang (ed.), and Methods in Molecular Biology, vol. 343: Agrobacterium Protocols, Vol. 1, Second Edition. Humana Press Inc., Totowa, NJ, pp. 15-41). In some embodiments, the selectable or screenable marker is selected from the group consisting of RFP, GUS, aadA, spectinomycin, streptomycin, and imazapyr. In some embodiments, the vector is a DICOTBINARY-19 plasmid. In some embodiments, the vector is a DICOTBINARY-22 plasmid.

In some aspects, provided herein are methods for transforming Cannabis using floral dip transformation. Female Cannabis flowers are exposed to Agrobacterium cultures suitable for flower dip transformation. In some embodiments, Cannabis flowers are submerged in an Agrobacterium culture under vacuum to induce transformation. In some embodiments, the Agrobacterium culture comprises Agrobacterium comprising the heterologous gene or nucleic acid of interest, a wetting agent, and a carrier. In some embodiments, the heterologous gene or nucleic acid of interest is included on a vector such as the DICOTBINARY-22 vector described herein. Other suitable vectors are known in the art. In some embodiments, the Agrobacterium strain is Ar18r12v, although other suitable Agrobacterium strains are known and used in the art. In some embodiments, the Agrobacterium used is a constitutively active variant or virG mutant of Agrobacterium (e.g., N45D mutant Agrobacterium). In some embodiments, the carrier is a cell culture medium suitable for the survival of the Agrobacterium. In some embodiments, the carrier is 5% sucrose. In some embodiments, the wetting agent is 0.05% silwet L-77. In some embodiments, the Agrobacterium culture includes Triton X-100, NP-40, Tween, or combinations thereof. In some embodiments, the Agrobacterium culture additionally comprises acetosyringone, galacturonic acid, cinnamic acid, coumarin, vanillin, other phenolic compounds, or combinations thereof. Suitable surfactants for plant floral dip transformations are known in the art. Following application of the Agrobacterium culture to the female Cannabis flower, the flowers are pollinated with male pollen from a suitable donor plant. In some embodiments, female Cannabis flowers are exposed to the Agrobacterium culture for at least 1, at least 2, at least 5, at least 10, at least 12, at least 15, at least 16, or at least 18 days before pollination. In some embodiments, the female Cannabis flowers are pollinated using a paintbrush.

In some embodiments of the floral dip transformations, DNA is delivered directly to the Cannabis ovules. In some embodiments, DNA is complexed with cell penetrating peptides prior to administration to Cannabis ovules.

In some aspects, provided herein are methods for transforming Cannabis nodes, internodes, leafs, petioles, hypocotyls, and buds. Sanitized and imbibed seeds are plated on non-selective medium (e.g., B5 medium) and grown for approximately 6 weeks or until explants suitable for transformation are formed. Resulting explants are inoculated with a heterologous gene or nucleic acid of interest. In some embodiments, the explants are inoculated using a force treatment as described herein. In some embodiments, the explants are inoculated by sonication with a vector comprising the heterologous gene or nucleic acid of interest. In some embodiment, the vector additionally comprises a selectable markers.

Following inoculation, node and bud explants are co-cultured in a culture medium that supports growth and survival of the node or bud for at least about 4 days. In some embodiments, the culture medium that supports the growth and survival of the node or bud is WCIC INO medium. In some embodiments, the medium additionally comprises nystatin, TBZ, and meta-topolin (mT). Following co-culture of at least about 4 days, the nodes and buds are transferred to a second culture medium suitable for the growth and survival of the nodes or buds. In some embodiments, the second culture medium is hemp node medium described herein in Table 7. In some embodiments, the hemp node medium additionally comprises a selection agent. In some embodiments, the hemp node medium additionally comprises activated charcoal.

Following inoculation, internode, leaf, hypocotyl, and petiole explants are co-cultured in a culture medium that supports growth and survival of the internode, leaf, hypocotyl, or petiole for at least about 4 days. In some embodiments, the culture medium that supports the growth and survival of the internode, leaf, hypocotyl, or petiole is WCIC INO medium. In some embodiments, the medium additionally comprises nystatin, TBZ, meta-topolin (mT), napthylacetic acid (NAA) and GA3. Following co-culture for at least about 4 days, leaf, petiole, hypocotyl, and internode explants are transferred to a second culture medium suitable for the growth and survival of the leaf, petiole, or internode. In some embodiments, the second culture medium is hemp internode medium described herein in Table 12. In some embodiments, the medium additionally includes a selection agent.

In some aspects, provided herein are methods for transforming Cannabis pollen or anther cultures. Cannabis pollen is harvested by shaking branches of male plants and collecting the pollen. In some embodiments, after harvest and collection, the pollen may be sized by passing the pollen through a sieve (e.g., a #80 sieve). Pollen may be used immediately for transformation, or may be stored prior to use. Pollen may be stored at a temperature between about 4° C. and about 20° C. The pollen may be stored in the presence of a storage medium. In some embodiments, the storage medium is medium suitable for pollen germination. In some embodiments, the storage medium includes boric acid, calcium chloride, potassium phosphate, and water. In some embodiments, the storage medium additionally includes glycerol.

Pollen may be transformed using particle bombardment, high velocity microprojection, microinjection, electroporation, direct DNA uptake, cell-penetrating peptides, silica carbide fibers, nanoparticles, and bacterially-mediated transformation. In some embodiments, pollen is transformed using silica carbide fibers. In some embodiments, pollen is transformed using cell-penetrating peptides. Following transformation, pollen is co-cultured and germinated on medium suitable for the survival of the pollen. The medium may include a suitable selection agent. In some embodiments, transformed pollen is used to pollenate female flowers for rapid generation of transgenic T1 progeny.

In some aspects, provided herein are methods for transforming Cannabis callus tissue or embryogenic suspension cells. In general, Cannabis leaf tissue is cultured on plant medium containing hormones suitable to induce embryogenic calli formation. Embryogenic calli are then transformed using any of the suitable transformation methods as described herein. Calli are then grown a suitable plant medium containing hormones suitable for induction of shooting in the plant. Suspension cells may similarly be transformed in suspension as single cells then subsequently grown into plants using suitable medium including suitable hormones. Suitable medium and hormones are known and described in the art.

The present invention has been described in terms of one or more preferred embodiments, and it should be appreciated that many equivalents, alternatives, variations, and modifications, aside from those expressly stated, are possible and within the scope of the invention.

### Example 1

The embodiment described here demonstrates Cannabis meristem explant transformation.

Seeds of variety Elektra x Chardonnay were surface sanitized with 20% Clorox for 5 minutes, rinsed, and sat for ˜2 hrs before overnight imbibition with WCIC Bean Germination Media (BGM).

TABLE 1
WCIC Bean Germination Media (BGM)

| | Amount |
|---|---|
| | to add |
| | per liter |
| Ingredients and Notes | (grams) |
| Phytotechnology Laboratories WPM L449 | 2.41 |
| Sucrose | 20 |
| pH to 5.8 with 1N KOH and autoclave | |
| Add the following prior to use: | |
| Captan fungicide (50WP) | 0.06 |
| Bravo fungicide (Daconil) (82DP) | 0.03 |
| Cefotaxime (100 mg/ml) | 1.25 ml |

The next day, meristem explants were aseptically excised from seed and incubated for approximately 2 hrs in 20% PEG4000 with 60 mg/L Captan and 30 mg/L Bravo fungicides. Explants were then rinsed and inoculated with Agrobacterium strain Ar18r12v harboring the binary plasmid DICOTBINARY-19. During inoculation, explants were exposed to 20 seconds of sonication at ˜45 kHz. Explants were co-cultured in either 1.75 ml or 2.0 ml WCIC INO media with 100 uM acetosyringone, 50 mg/L nystatin, 10 mg/L TBZ, and 95 uM lipoic acid for 4 days at 23 C 16/8 photoperiod. Thidiazuron (TDZ) was added to some co-culture treatments at 1 mg/L.

TABLE 2
WCIC INO media

| | Amount |
|---|---|
| | to add |
| | per liter |
| Ingredients and Notes | (grams) |
| Gamborg B5 Phytotechnology | 1.284 |
| Laboratories G398 | |
| Glucose | 30 |
| MES | 2.8 |
| pH to 5.4 with 1N KOH and | |
| autoclave | |

After 4 days of co-culture, transient GUS expression was evaluated in explants. Explants were then transferred to either 10 mg/L spectinomycin or 150 mg/L spectinomycin WCIC Gamborg B5 medium (Table 3) for selection. Explants on 10 mg/L spectinomycin B5 were transferred to 150 mg/L spectinomycin B5 approximately 1 week later, and then explants from all treatments were transferred to 50 mg/L spectinomycin B5 1 month later. Additional transfers have been made with explants remaining green on spectinomycin. A summary of this experiment is given in Table 4.

TABLE 3
WCIC Gamborg B5 Medium

| | Amount to add per liter |
|---|---|
| Ingredients and Notes | (grams) |
| Phytotechnology Laboratories | 2.41 |
| B5 salts G398 | |
| Sucrose | 20 |
| Cleary's 3336 (50WP) | 0.06 |
| Ca Gluconate | 1.29 |
| pH to 5.8 with 1N KOH | |
| Phytagel | 3.50 |
| autoclave | |
| Add the following fresh before use: | |
| Timetin (150 mg/ml stock) | Use 1 mL per Liter (150 mg/L) |
| Cefotaxime (100 mg/ml stock) | Use at 2 ml per Liter (200 mg/L) |
| Carbenicillin (100 mg/ml stock) | Use at 4 ml per Liter (400 mg/L) |
| Selective Agent | as needed |

TABLE 4
Description and summary of Cannabis meristem explant transformation experiments.

| | | # | | | | | |
|---|---|---|---|---|---|---|---|
| | | embryos | | | | | |
| | Experiment | to | Selection | | | | |
| Co-Culture conditions | ID | Selection | Media | 2nd media | 3rd media | Notes | Notes |
| Filter paper in plantcon | Hemp 3/22- | 7 | 10 ppm spec | 150 ppm spec | 50 ppm spec | 1 greening explant | |
| with 1.75 ml INO + 50 | 1A | | B5; 28 C. 16/8 | B5; 28 C. 16/8 | B5; 28 C. 16/8 | transferred to non- | |
| ppm nystatin + 10 ppm | | | photoperiod; | photoperiod; | photoperiod; | selective BRM | |
| TBZ + lipoic acid; 23 C. | | | | | | | |
| 16/8 photoperiod | | | | | | | |
| Filter paper in plantcon | Hemp 3/22- | 4 | 150 ppm spec | NA | 50 ppm spec | 1 greening explant | |
| with 1.75 ml INO + 50 | 1B | | B5; 28 C. 16/8 | | B5; 28 C. 16/8 | transferred to non- | |
| ppm nystatin + 10 ppm | | | photoperiod; | | photoperiod; | selective BRM | |
| TBZ + lipoic acid; 23 C. | | | | | | | |
| 16/8 photoperiod | | | | | | | |
| Filter paper in plantcon | Hemp 3/22- | 9 | 10 ppm spec | 150 ppm spec | 50 ppm spec | 1 greening explant | |
| with 2 ml INO + 50 ppm | 2A | | B5; 28 C. 16/8 | B5; 28 C. 16/8 | B5; 28 C. 16/8 | transferred to 10 | |
| nystatin + 10 ppm TBZ + | | | photoperiod; | photoperiod; | photoperiod; | ppm spec B5 | |
| lipoic acid; 23 C. 16/8 | | | | | | | |
| photoperiod | | | | | | | |
| Filter paper in plantcon | Hemp 3/22- | 10 | 150 ppm spec | NA | 50 ppm spec | 1 chimeric RFP+ | 1 greening explant |
| with 2 ml INO + 50 ppm | 2B | | B5; 28 C. 16/8 | | B5; 28 C. 16/8 | explant (primary | transferred to non- |
| nystatin + 10 ppm TBZ + | | | photoperiod; | | photoperiod; | leaves) transferred | selective BRM |
| lipoic acid; 23 C. 16/8 | | | | | | to B5 | |
| photoperiod | | | | | | | |
| Filter paper in plantcon | Hemp 3/22- | 9 | 10 ppm spec | 150 ppm spec | 50 ppm spec | | |
| with 1.75 ml INO + 50 | 3A | | B5; 28 C. 16/8 | B5; 28 C. 16/8 | B5; 28 C. 16/8 | | |
| ppm nystatin + 10 ppm | | | photoperiod; | photoperiod; | photoperiod; | | |
| TBZ + lipoic acid + 1 | | | | | | | |
| ppm TDZ; 23 C. 16/8 | | | | | | | |
| photoperiod | | | | | | | |
| Filter paper in | Hemp 3/22- | 10 | 150 ppm | NA | 50 ppm spec | 1 chimeric RFP + | 4 greening explant |
| plantcon with 1.75 ml | 3B | | spec B5; 28 C. | | B5; 28 C. 16/8 | explant (primary | transferred to non- |
| INO + 50 ppm nystatin + | | | 16/8 | | photoperiod; | leaves) | selective BRM; |
| 10 ppm TBZ + lipoic | | | photoperiod; | | | transferred to B5 | GUS positive leaf |
| acid + 1 ppm TDZ; | | | | | | | imaged |
| 23 C. 16/8 photoperiod | | | | | | | |
| Filter paper in | Hemp 3/22- | 9 | 10 ppm spec | 150 ppm | 50 ppm spec | RFP positive | |
| plantcon with 2 ml | 4A | | B5; 28 C. 16/8 | spec B5; 28 C. | B5; 28 C. 16/8 | shoot imaged | |
| INO + 50 ppm nystatin + | | | photoperiod; | 16/8 | photoperiod; | | |
| 10 ppm TBZ + lipoic | | | | photoperiod; | | | |
| acid + 1 ppm TDZ; | | | | | | | |
| 23 C. 16/8 photoperiod | | | | | | | |
| Filter paper in plantcon | Hemp 3/22- | 12 | 150 ppm spec | NA | 50 ppm spec | 5 greening explants | |
| with 2 ml INO + 50 ppm | 4B | | B5; 28 C. 16/8 | | B5; 28 C. 16/8 | transferred to 10 | |
| nystatin + 10 ppm TBZ + | | | photoperiod; | | photoperiod; | ppm spec B5 | |
| lipoic acid + 1 ppm | | | | | | | |
| TDZ; 23 C. 16/8 | | | | | | | |
| photoperiod | | | | | | | |

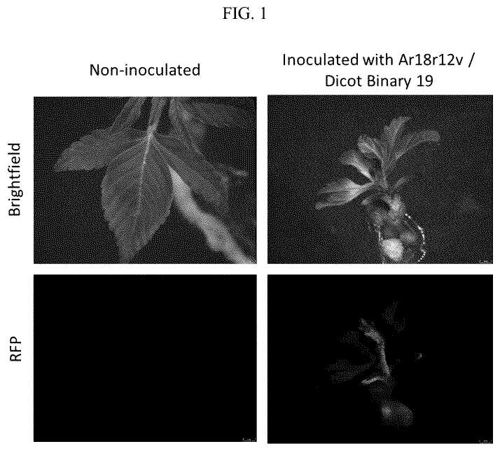

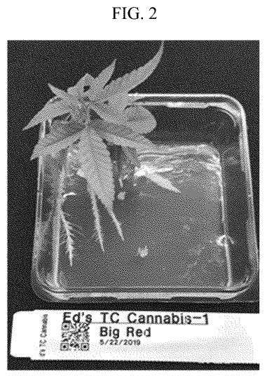

Stable RFP (tdTomato) was imaged several explants 3 weeks after inoculation, but was confined primarily to primary leaves and cotyledonary remnants. The stable RFP signal in the chimeric plantlet that appeared to be from new growth was from treatment 4A (treatment bolded in Table 4-2 ml co-culture volume with TDZ, and initial transfer to 10 mg/L spectinomycin B5), shown again in FIG. 1. This plant did not survive tissue culture, which may have been result of its chimerism in response to selection, or possibly non-optimal media conditions.

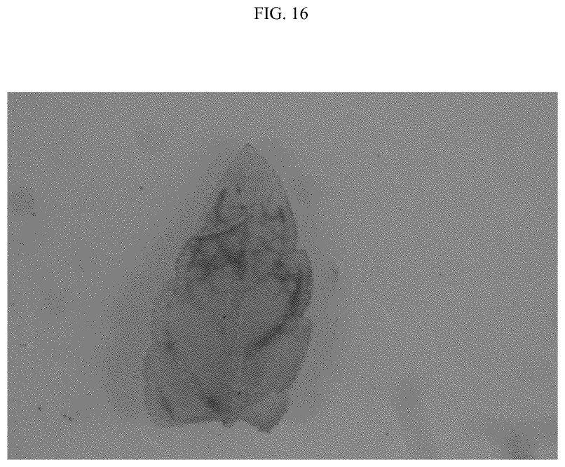

We have also observed stable GUS expression in a plantlet derived from this experiment in treatment Hemp 3/22-3B. One of the leaves in a plantlet transferred to non-selective BRM expressed GUS in leaf stably. The leaf shown in FIG. 16 was imaged 2 months after inoculation after clearing with 70% ethanol.

The non-inoculated control meristem explant was sent to the greenhouse as a proof of concept of tissue culture (TC) regeneration of a plant from a meristem explant (rooted on non-selective B5 media). See FIG. 2.

Additional experiments with additional varieties of Cannabis inoculated with Ar18r12v/DICOTBINARY-19 and a 4 day co-culture are outlined in Table 5. The co-culture volume was increased as we noted in the prior tests explants were very dry post co-culture. Some of these experiments used meta-topolin in co-culture, which has been demonstrated to encourage propagation in Cannabis nodal cultures (H. Lata et al./Journal of Applied Research on Medicinal and Aromatic Plants 3 (2016) 18-26); some used a full-strength formulation of B5 media for selection; and some used an MS-based selection media with meta-topolin (mT) based on Lata 2016 but without activated charcoal.

TABLE 5
Description and summary of follow-up experiments with Cannabis meristem explants.

| Cannabis | | | | | # | |
|---|---|---|---|---|---|---|
| sativa | | Experi- | | | embryos | |
| Genotype/ | | ment | | | to | |
| Line | Comments | ID | Strain | Binary | Selection | Notes |
| Honey | Hand excised from seed surface sanitized in 20% Clorox 5 min; | Hemp | Ar18r12v | DICOT- | 35 | 10 ppm spec |
| Gold | rinsed; imbibed for ~20 h in BGM; explants placed in 20% | 5/9-1 | | BINARY- | | 100% B5; |
| 3WS | PEG4000 with Captan/Bravo for 1.5-2.5 h, rinsed, inoculated | | | 19 | | 28 C. 16/8 |
| | and sonicated 20 s 45 kHz; incubated 30 min, inoculum | | | | | photoperiod; |
| | removed; explants co-cultured on filter paper in plantcon with | | | | | |
| | 2.5 ml INO + 60 ppm Cleary's + 50 ppm nystatin + 10 ppm | | | | | |
| | TBZ; 23 C. 16/8 photoperiod | | | | | |
| Honey | Hand excised from seed surface sanitized in 20% Clorox 5 min; | Hemp | Ar18r12v | DICOT- | 25 | 10 ppm spec |
| Gold | rinsed; imbibed for ~20 h in BGM; explants placed in 20% | 5/9-2 | | BINARY- | | 100% B5; |
| 3WS | PEG4000 with Captan/Bravo for 1.5-2.5 h, rinsed, inoculated | | | 19 | | 28 C. 16/8 |
| | and sonicated 20 s 45 kHz; incubated 30 min, inoculum | | | | | photoperiod; |
| | removed; explants co-cultured on filter paper in plantcon with | | | | | |
| | 2.5 ml INO + 60 ppm Cleary's + 50 ppm nystatin + 10 ppm | | | | | |
| | TBZ + 1 ppm meta-topolin (mT); 23 C. 16/8 photoperiod | | | | | |
| Fiber | Hand excised from seed surface sanitized in 20% Clorox 5 min; | Hemp | Ar18r12v | DICOT- | 21 | 10 ppm spec |
| Hemp | rinsed; imbibed for ~20 h in BGM; explants placed in 20% | 5/9-3 | | BINARY- | | 100% B5; |
| | PEG4000 with Captan/Bravo for 1.5-2.5 h, rinsed, inoculated | | | 19 | | 28 C. 16/8 |
| | and sonicated 20 s 45 kHz; incubated 30 min, inoculum | | | | | photoperiod; |
| | removed; explants co-cultured on filter paper in plantcon with | | | | | |
| | 2.5 ml INO + 60 ppm Cleary's + 50 ppm nystatin + 10 ppm | | | | | |
| | TBZ; 23 C. 16/8 photoperiod | | | | | |
| Fiber | Hand excised from seed surface sanitized in 20% Clorox 5 min; | Hemp | Ar18r12v | DICOT- | 12 | 10 ppm spec |
| Hemp | rinsed; imbibed for ~20 h in BGM; explants placed in 20% | 5/9-4 | | BINARY- | | 100% B5; |
| | PEG4000 with Captan/Bravo for 1.5-2.5 h, rinsed, inoculated | | | 19 | | 28 C. 16/8 |
| | and sonicated 20 s 45 kHz; incubated 30 min, inoculum | | | | | photoperiod; |
| | removed; explants co-cultured on filter paper in plantcon with | | | | | |
| | 2.5 ml INO + 60 ppm Cleary's + 50 ppm nystatin + 10 ppm | | | | | |
| | TBZ + 1 ppm meta-topolin (mT); 23 C. 16/8 photoperiod | | | | | |
| Honey | Hand excised from seed surface sanitized in 20% Clorox 5 min; | Hemp | Ar18r12v | DICOT- | 59 | 10 ppm spec |
| Gold | rinsed; imbibed for ~20 h in BGM; explants placed in 20% | 5/16-1 | | BINARY- | | 100% B5; |
| 3WS | PEG4000 with Captan/Bravo for 1.5-2.5 h, rinsed, inoculated | | | 19 | | 28 C. 16/8 |
| | and sonicated 20 s 45 kHz; incubated 30 min, inoculum | | | | | photoperiod; |
| | removed; explants co-cultured on filter paper in plantcon with | | | | | |
| | 2.5 ml INO + 60 ppm Cleary's + 50 ppm nystatin + 10 ppm | | | | | |
| | TBZ 1 ppm TDZ; 23 C. 16/8 photoperiod | | | | | |
| Honey | Hand excised from seed surface sanitized in 20% Clorox 5 min; | Hemp | Ar18r12v | DICOT- | 19 | 10 ppm spec |
| Gold | rinsed; imbibed for ~20 h in BGM; explants placed in 20% | 5/16-2 | | BINARY- | | 100% B5; |
| 3WS | PEG4000 with Captan/Bravo for 1.5-2.5 h, rinsed, inoculated | | | 19 | | 28 C. 16/8 |
| | and sonicated 20 s 45 kHz; incubated 30 min, inoculum | | | | | photoperiod; |
| | removed; explants co-cultured on filter paper on top of | | | | | |
| | semisolid INO (8 g/L agarose I) + 60 ppm Cleary's + 1 ppm | | | | | |
| | TDZ; 23 C. 16/8 photoperiod | | | | | |
| Honey | Hand excised from seed surface sanitized in 20% Clorox 5 min; | Hemp | Ar18r12v | DICOT- | 5 | non- |
| Gold | rinsed; imbibed for ~20 h in BGM; explants placed in 20% | 5/30-1 | | BINARY- | | selective |
| 3WS | PEG4000 with Captan/Bravo for 1.5-2.5 h, rinsed, inoculated | | | 19 | | hemp node |
| | and sonicated 20 s 45 kHz; incubated 30 min, inoculum | | | | | media |
| | removed; explants co-cultured on filter paper in plantcon with | | | | | (minus |
| | 2.5 ml INO + 60 ppm Cleary's + 50 ppm nystatin + 10 ppm | | | | | activated |
| | TBZ 1 ppm TDZ; 23 C. 16/8 photoperiod | | | | | charcoal) |
| Honey | Hand excised from seed surface sanitized in 20% Clorox 5 min; | Hemp | Ar18r12v | DICOT- | 36 | 10 ppm sec |
| Gold | rinsed; imbibed for ~20 h in BGM; explants placed in 20% | 5/30-2 | | BINARY- | | hemp node |
| 3WS | PEG4000 with Captan/Bravo for 1.5-2.5 h, rinsed, inoculated | | | 19 | | media |
| | and sonicated 20 s 45 kHz; incubated 30 min, inoculum | | | | | (minus |
| | removed; explants co-cultured on filter paper in plantcon with | | | | | activated |
| | 2.5 ml INO + 60 ppm Cleary's + 50 ppm nystatin + 10 ppm | | | | | charcoal) |
| | TBZ 1 ppm TDZ; 23 C. 16/8 photoperiod | | | | | |
| Honey | Hand excised from seed surface sanitized in 20% Clorox 5 min; | Hemp | Ar18r12v | DICOT- | 43 | 50 ppm sec |
| Gold | rinsed; imbibed for ~20 h in BGM; explants placed in 20% | 5/30-3 | | BINARY- | | hemp node |
| 3WS | PEG4000 with Captan/Bravo for 1.5-2.5 h, rinsed, inoculated | | | 19 | | media |
| | and sonicated 20 s 45 kHz; incubated 30 min, inoculum | | | | | (minus |
| | removed; explants co-cultured on filter paper in plantcon with | | | | | activated |
| | 2.5 ml INO + 60 ppm Cleary's + 50 ppm nystatin + 10 ppm | | | | | charcoal) |
| | TBZ + 1 ppm TDZ; 23 C. 16/8 photoperiod | | | | | |
| Honey | Hand excised from seed surface sanitized in 20% Clorox 5 min; | Hemp | Ar18r12v | SOYTEST-2 | 36 | 50 ppm sec |
| Gold | rinsed; imbibed for ~20 h in BGM at 37 C.; explants placed in | 6/6-1 | | | | hemp node |
| 3WS | 20% PEG4000 with Captan/Bravo for 1.5-2.5 h, rinsed, | | | | | media |
| | inoculated and sonicated 20 s 45 kHz; incubated 30 min, | | | | | (minus |
| | inoculum removed; explants co-cultured on filter paper in | | | | | activated |
| | plantcon with 2.5 ml INO + 60 ppm Cleary's + 50 ppm | | | | | charcoal) |
| | nystatin + 10 ppm TBZ + 1 ppm TDZ; 23 C. 16/8 photoperiod | | | | | |
| Honey | Hand excised from seed surface sanitized in 20% Clorox 5 min; | Hemp | Ar18r12v | SOYTEST-2 | 36 | 50 ppm sec |
| Gold | rinsed; imbibed for ~20 h in BGM at 37 C.; explants placed in | 6/6-2 | | | | hemp node |
| 3WS | 20% PEG4000 with Captan/Bravo for 1.5-2.5 h, rinsed, | | | | | media |
| | inoculated and sonicated 2 min 45 kHz; incubated 30 min, | | | | | (minus |
| | inoculum removed; explants co-cultured on filter paper in | | | | | activated |
| | plantcon with 2.5 ml INO + 60 ppm Cleary's + 50 ppm | | | | | charcoal) |
| | nystatin + 10 ppm TBZ + 1 ppm TDZ; 23 C. 16/8 photoperiod | | | | | |
| Honey | Hand excised from seed surface sanitized in 20% Clorox 5 min; | Hemp | Ar18r12v | SOYTEST-2 | 44 | 50 ppm sec |
| Gold | rinsed; imbibed for ~20 h in BGM at 37 C.; explants placed in | 6/13-1 | | | | hemp node |
| 3WS | 20% PEG4000 with Captan/Bravo for 1.5-2.5 h, rinsed, | | | | | media |
| | inoculated and sonicated 20 s 45 kHz; incubated 30 min, | | | | | (minus |
| | inoculum removed; explants co-cultured on filter paper in | | | | | activated |
| | plantcon with 2.5 ml INO + 50 ppm nystatin + 10 ppm TBZ + 1 | | | | | charcoal) |
| | ppm TDZ; 23 C. 16/8 photoperiod | | | | | |
| Honey | Hand excised from seed surface sanitized in 20% Clorox 5 min; | Hemp | Ar18r12v | SOYTEST-2 | 45 | 50 ppm sec |
| Gold | rinsed; imbibed for ~20 h in BGM at 37 C.; explants placed in | 6/13-2 | | | | hemp node |
| 3WS | 20% PEG4000 with Captan/Bravo for 1.5-2.5 h, rinsed, | | | | | media |
| | inoculated and sonicated 2 min 45 kHz; incubated 30 min, | | | | | (minus |
| | inoculum removed; explants co-cultured on filter paper in | | | | | activated |
| | plantcon with 2.5 ml INO + 50 ppm nystatin + 10 ppm TBZ + 1 | | | | | charcoal) |
| | ppm TDZ; 23 C. 16/8 photoperiod | | | | | |
| Honey | Hand excised from seed surface sanitized in 20% Clorox 5 min; | Hemp | Ar18r12v | SOYTEST-2 | 20 | 10 ppm sec |
| Gold | rinsed; imbibed for ~20 h in BGM at 37 C.; explants placed in | 6/20-1A | | | | B5; 28 C. |
| 3WS | 20% PEG4000 with Captan/Bravo for 1.5-2.5 h, rinsed, | | | | | 16/8 |
| | inoculated and sonicated 20 s 45 kHz; incubated 30 min, | | | | | photocopied; |
| | inoculum removed; explants co-cultured on filter paper in | | | | | |
| | plantcon with 2.5 ml INO + 60 ppm Cleary's + 50 ppm | | | | | |
| | nystatin + 10 ppm TBZ; 23 C. 16/8 photoperiod | | | | | |
| Honey | Hand excised from seed surface sanitized in 20% Clorox 5 min; | Hemp | Ar18r12v | SOYTEST-2 | 10 | 10 ppm sec |
| Gold | rinsed; imbibed for ~20 h in BGM at 37 C.; explants placed in | 6/20-1B | | | | node-AC; |
| 3WS | 20% PEG4000 with Captan/Bravo for 1.5-2.5 h, rinsed, | | | | | 28 C. 16/8 |
| | inoculated and sonicated 20 s 45 kHz; incubated 30 min, | | | | | photocopied; |
| | inoculum removed; explants co-cultured on filter paper in | | | | | |
| | plantcon with 2.5 ml INO + 60 ppm Cleary's + 50 ppm | | | | | |
| | nystatin + 10 ppm TBZ; 23 C. 16/8 photoperiod | | | | | |
| Honey | Hand excised from seed surface sanitized in 20% Clorox 5 min; | Hemp | Ar18r12v | SOYTEST-2 | 10 | 50 ppm sec |
| Gold | rinsed; imbibed for ~20 h in BGM at 37 C.; explants placed in | 6/20-1C | | | | B5; 28 C. |
| 3WS | 20% PEG4000 with Captan/Bravo for 1.5-2.5 h, rinsed, | | | | | 16/8 |
| | inoculated and sonicated 20 s 45 kHz; incubated 30 min, | | | | | photocopied; |
| | inoculum removed; explants co-cultured on filter paper in | | | | | |
| | plantcon with 2.5 ml INO + 60 ppm Cleary's + 50 ppm | | | | | |
| | nystatin + 10 ppm TBZ; 23 C. 16/8 photoperiod | | | | | |
| Honey | Hand excised from seed surface sanitized in 20% Clorox 5 min; | Hemp | Ar18r12v | SOYTEST-2 | 10 | 50 ppm strep |
| Gold | rinsed; imbibed for ~20 h in BGM at 37 C.; explants placed in | 6/20-1D | | | | node-AC; |
| 3WS | 20% PEG4000 with Captan/Bravo for 1.5-2.5 h, rinsed, | | | | | 28 C. 16/8 |
| | inoculated and sonicated 20 s 45 kHz; incubated 30 min, | | | | | photoperiod; |
| | inoculum removed; explants co-cultured on filter paper in | | | | | |
| | plantcon with 2.5 ml INO + 60 ppm Cleary's + 50 ppm | | | | | |
| | nystatin + 10 ppm TBZ; 23 C. 16/8 photoperiod | | | | | |
| Honey | Hand excised from seed surface sanitized in 20% Clorox 5 min; | Hemp | Ar18r12v | SOYTEST-2 | 10 | 10 ppm spec |
| Gold | rinsed; imbibed for ~20 h in BGM at 37 C.; explants placed in | 6/20-2A | | | | B5; 28 C. |
| 3WS | 20% PEG4000 with Captan/Bravo for 1.5-2.5 h, rinsed, | | | | | 16/8 |
| | inoculated and sonicated 20 s 45 kHz; incubated 30 min, | | | | | photoperiod; |
| | inoculum removed; explants co-cultured on filter paper in | | | | | |
| | plantcon with 2.5 ml INO + 1 ppm TDZ, 60 ppm Cleary's + 50 | | | | | |
| | ppm nystatin + 10 ppm TBZ; 23 C. 16/8 photoperiod | | | | | |
| Honey | Hand excised from seed surface sanitized in 20% Clorox 5 min; | Hemp | Ar18r12v | SOYTEST-2 | 10 | 10 ppm spec |
| Gold | rinsed; imbibed for ~20 h in BGM at 37 C.; explants placed in | 6/20-2B | | | | node-AC; |
| 3WS | 20% PEG4000 with Captan/Bravo for 1.5-2.5 h, rinsed, | | | | | 28 C. 16/8 |
| | inoculated and sonicated 20 s 45 kHz; incubated 30 min, | | | | | photoperiod; |
| | inoculum removed; explants co-cultured on filter paper in | | | | | |
| | plantcon with 2.5 ml INO + 1 ppm TDZ, 60 ppm Cleary's + 50 | | | | | |
| | ppm nystatin + 10 ppm TBZ; 23 C. 16/8 photoperiod | | | | | |
| Honey | Hand excised from seed surface sanitized in 20% Clorox 5 min; | Hemp | Ar18r12v | SOYTEST-2 | 10 | 50 ppm spec |
| Gold | rinsed; imbibed for ~20 h in BGM at 37 C.; explants placed in | 6/20-2C | | | | B5; 28 C. |
| 3WS | 20% PEG4000 with Captan/Bravo for 1.5-2.5 h, rinsed, | | | | | 16/8 |
| | inoculated and sonicated 20 s 45 kHz; incubated 30 min, | | | | | photoperiod; |
| | inoculum removed; explants co-cultured on filter paper in | | | | | |
| | plantcon with 2.5 ml INO + 1 ppm TDZ, 60 ppm Cleary's + 50 | | | | | |
| | ppm nystatin + 10 ppm TBZ; 23 C. 16/8 photoperiod | | | | | |
| Honey | Hand excised from seed surface sanitized in 20% Clorox 5 min; | Hemp | Ar18r12v | SOYTEST-2 | 10 | 50 ppm strep |
| Gold | rinsed; imbibed for ~20 h in BGM at 37 C.; explants placed in | 6/20-2D | | | | node-AC; |
| 3WS | 20% PEG4000 with Captan/Bravo for 1.5-2.5 h, rinsed, | | | | | 28 C. 16/8 |
| | inoculated and sonicated 20 s 45 kHz; incubated 30 min, | | | | | photoperiod; |
| | inoculum removed; explants co-cultured on filter paper in | | | | | |
| | plantcon with 2.5 ml INO + 1 ppm TDZ, 60 ppm Cleary's + 50 | | | | | |
| | ppm nystatin + 10 ppm TBZ; 23 C. 16/8 photoperiod | | | | | |
| Honey | Hand excised from seed surface sanitized in 20% Clorox 5 min; | Hemp | GV3101 | SOYTEST-2 | 48 | 50 ppm spec |
| Gold | rinsed; imbibed for ~20 h in BGM at 37 C.; explants placed in | 6/27-1 | | | | meristem |
| 3WS | 20% PEG4000 with Captan/Bravo for 1.5-2.5 h, rinsed, | | | | | regeneration; |
| | inoculated and sonicated 20 s 45 kHz; incubated 30 min, | | | | | 28 C. 16/8 |
| | inoculum removed; explants co-cultured on filter paper in | | | | | photoperiod; |
| | plantcon with 2.25 ml INO + 60 ppm Cleary's + 50 ppm | | | | | |
| | nystatin + 10 ppm TBZ + 1 ppm TDZ; 23 C. 16/8 photoperiod | | | | | |
| Honey | Hand excised from seed surface sanitized in 20% Clorox 5 min; | Hemp | GV3101 | SOYTEST-2 | 36 | 50 ppm spec |
| Gold | rinsed; imbibed for ~20 h in BGM at 37 C.; explants placed in | 6/27-2 | | | | meristem |
| 3WS | 20% PEG4000 with Captan/Bravo for 1.5-2.5 h, rinsed, | | | | | regeneration |
| | inoculated and sonicated 20 s 45 kHz; incubated 30 min, | | | | | with 0.5 |
| | inoculum removed; explants co-cultured on filter paper in | | | | | ppm MT; |
| | plantcon with 2.25 ml INO + 60 ppm Cleary's + 50 ppm | | | | | 28 C. 16/8 |
| | nystatin + 10 ppm TBZ + 1 ppm TDZ; 23 C. 16/8 photoperiod | | | | | photoperiod; |
| Honey | Hand excised from seed surface sanitized in 20% Clorox 5 min; | Hemp | Ar18r12v | SOYTEST-2 | 83 | 10 ppm spec |
| Gold | rinsed; imbibed for ~20 h in BGM at 37 C.; explants placed in | 7/3-1 | | | | meristem |
| 3WS | 20% PEG4000 with Captan/Bravo for 1.5-2.5 h, rinsed, | | | | | regeneration; |
| | inoculated and sonicated 20 s 45 kHz; incubated 30 min, | | | | | 28 C. 16/8 |
| | inoculum removed; explants co-cultured on filter paper in | | | | | photoperiod; |
| | plantcon with 2.25 ml INO + 60 ppm Cleary's + 1 ppm TDZ; | | | | | |
| | 23 C. 16/8 photoperiod | | | | | |
| Honey | Hand excised from seed surface sanitized in 20% Clorox 5 min; | Hemp | Ar18r12v | SOYTEST-2 | 48 | 50 ppm spec |
| Gold | rinsed; imbibed for ~20 h in BGM at 37 C.; explants placed in | 7/8-1 | | | | meristem |
| 3WS | 20% PEG4000 with Captan/Bravo for 1.5-2.5 h, rinsed, | | | | | regeneration |
| | inoculated and sonicated 20 s 45 kHz; incubated 30 min, | | | | | with 0.5 |
| | inoculum removed; explants co-cultured on filter paper on | | | | | ppm mT; |
| | semisolid INO (8 g/L agarose I) + 60 ppm Cleary's 1 ppm TDZ; | | | | | 28 C. 16/8 |
| | 23 C. 16/8 photoperiod | | | | | photoperiod; |

TABLE 6
WCIC TRUE Gamborg B5 Media

| Ingredients and Notes | Amount to add per liter (grams) |
|---|---|
| Phytotechnology Laboratories B5 | 3.21 |
| salts G398 | |
| Sucrose | 20 |
| Cleary's 3336 (50WP) | 0.06 |
| Ca Gluconate | 1.29 |
| pH to 5.8 with 1N KOH | |
| Phytagel | 3.50 |
| autoclave | |
| Add the following fresh before use: | |
| Timetin (150 mg/ml stock) | Use 1 mL per Liter (150 mg/L) |
| Cefotaxime (100 mg/ml stock) | Use at 2 ml per Liter (200 mg/L) |
| Carbenicillin (100 mg/ml stock) | Use at 4 ml per Liter (400 mg/L) |
| Selective Agent | as needed |

TABLE 7
WCIC Hemp Node Media (modified from Lata 2016)

| Ingredients and Notes | Amount to add per liter (grams) |
|---|---|
| MS Salts complete with vitamins | 4.43 |
| (PhytoTech M519) | |
| Sucrose | 30 |
| Cleary's 3336 | 0.06 |
| pH to 5.7 with 1N KOH | |
| Agar (Sigma A7921) | 8 |
| autoclave | |
| Meta-topolin (mT) (1 mg/ml) | 0.5 ml |
| Carbenicillin (200 mg/ml) | 1.25 ml |
| Cefotaxime (100 mg/ml) | 2 ml |
| Selection | as needed |

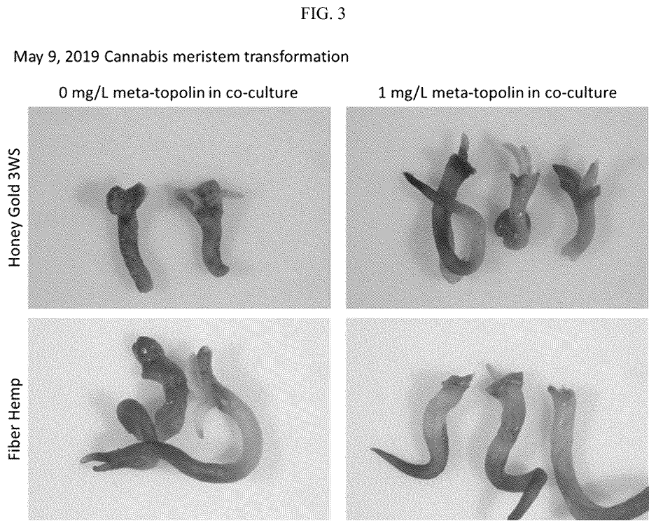

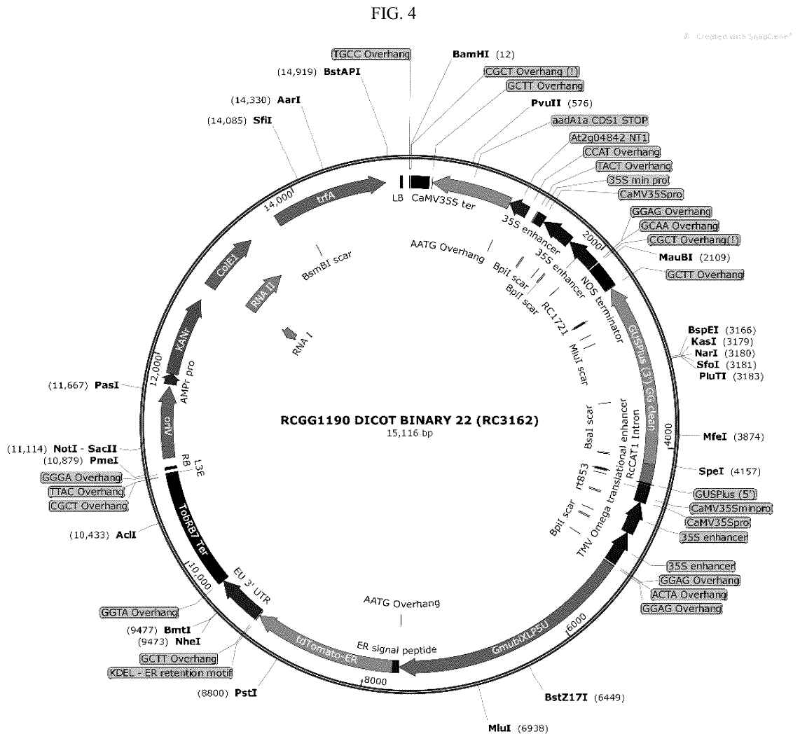

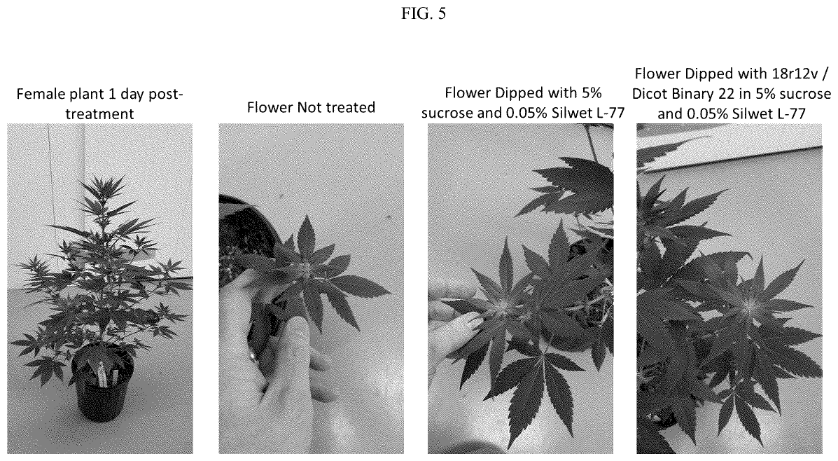

We did obtain strong GUS transients using both Cannabis varieties Honey Gold 3WS and the Fiber Hemp (in addition to the transients shown for Elektra x Chardonnay in initial disclosure). See FIG. 3.

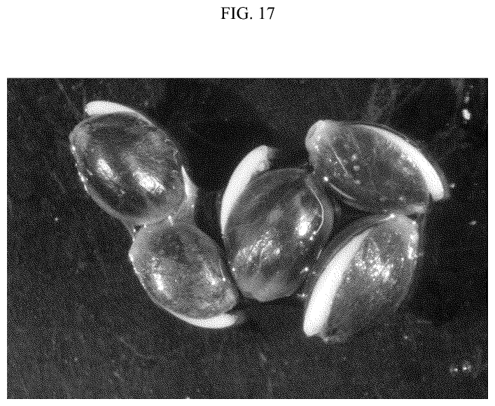

Additionally, Cannabis seeds were also imbibed at 37° C. to facilitate excision of the meristem explants. With an overnight imbibition at 37° C. the radical begins to emerge from the seed which makes a natural crack in the hard seed coat. This makes isolating the Cannabis mature embryo/meristem explant easier. See FIG. 17.

It is also possible to mechanically excise the Cannabis meristem explants from the seen. 10 g Cannabis seed from the Fiber variety were surface sterilized with 20% bleach solution for 5 minutes, then rinsed with sterile distilled water for 2 minutes. Seed were then imbibed in sterile distilled water at 37 degrees C. for approximately 24 hours. Seed was then rinsed with sterile distilled water for 2 minutes and weighed (collected rehydrated weight was 18 grams). Seed was then split into two 9 gram samples and spread on sterile filter paper in petri dishes and dried in laminar flow hood. One sample was removed 24hrs later, the other 49 hours later. The collected dry weight of each sample was 4.8 grams.

A portion of this dry hemp seed from the 49 hour dried material was used for machine excision experiments. Seed were placed through a Perten Instruments Laboratory Mill 3310 using seven different gap settings (0 to 6, smallest to largest gap) with 20 seeds per gap setting. Ground material was collected and embryonic parts were counted under a microscope. While the Perten Lab mill was used for these examples, excision of embryos and explants can be performed using and dry mill or equivalent instrumentation known in the art, for example, roller mills, hammer mills, and bladed mills or other suitable means described herein. Equivalent wet mill processing is also envisioned.

TABLE 8
Embryonic parts produced using various gap
setting on the Perten Instruments Laboratory Mill 3310

| Gap Setting | Embryonic Parts Produced |
|---|---|
| 6 | 20 |
| 5 | 14 |
| 4 | 14 |
| 3 | 5 |
| 2 | 3 |
| 1 | 4 |
| 0 | 0 |

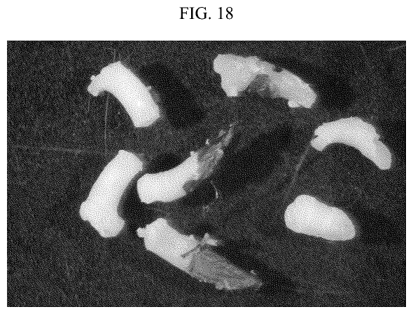

Further experimentation will be carried out using gap setting 6 for grinding hemp varieties. Following embryonic part production, regeneration of the embryonic material will be checked on non-selective medium. Embryonic parts generated using the gap 6 setting on Perten is shown in FIG. 18.

Additionally, MS-based medium with or without meta-topolin may be used with meristem explants. When TDZ is used in co-culture with Agrobacterium, the use of meta-topolin during selection/regeneration may not be necessary.

TABLE 9
Hemp Meristem Regeneration Medium

| Ingredients and Notes | Amount to add per liter (grams) |
|---|---|
| MS Salts complete with vitamins | 4.43 |
| (PhytoTech M519) | |
| Sucrose | 30 |
| Cleary's 3336 | 0.06 |
| pH to 5.7 with 1N KOH | |
| Agar (Sigma A7921) | 8 |
| autoclave | |
| Meta-topolin (mT) (1 mg/ml) | as needed |
| Carbenicillin (100 mg/ml) | 2 ml |
| Cefotaxime (100 mg/ml) | 2 ml |
| Timetin (150 mg/ml) | 1 ml |
| Selection | as needed |

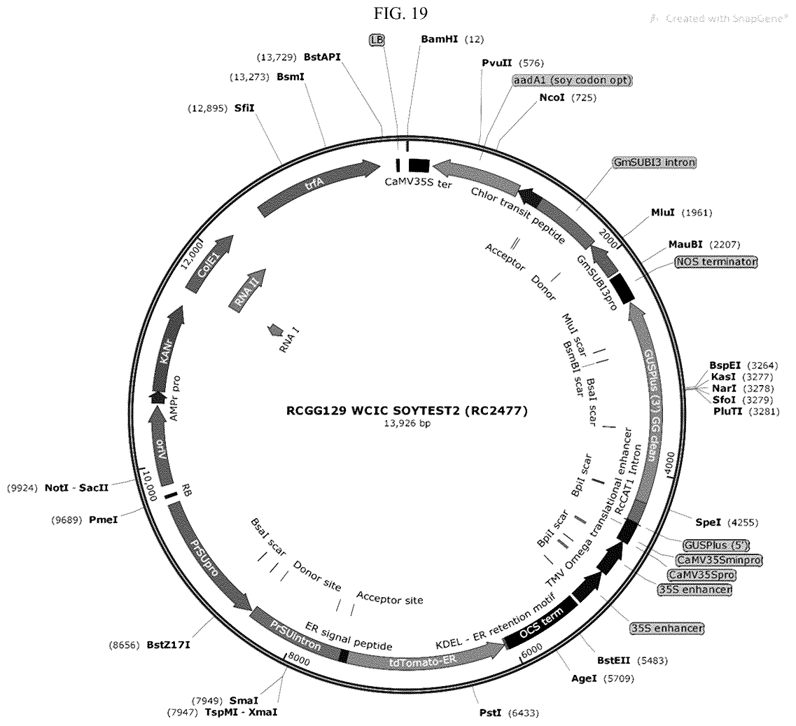

Additional control binary constructs have also been testing including SOYTEST-2. SOYTEST-2 has a different promoter driving tdTomato to test impacts on RFP visualization (DICOTBINARY-19 uses the Glycine max Ubiquitin 3 XL promoter driving tdTomato, where SOYTEST-2 uses Pinus radiata Super Ubiquitin promoter driving tdTomato). See FIG. 19.

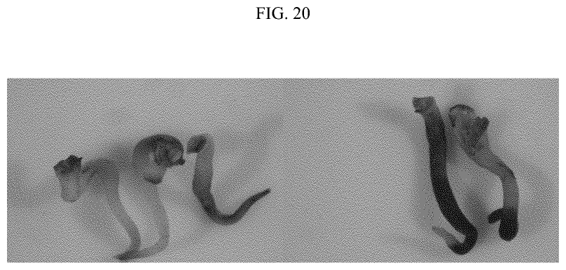

We have obtained positive GUS transients in Cannabis meristem explants (3WS variety) using SOYTEST-2 in both the Ar18r12v and GV3101 strains of Agrobacterium (see FIG. 20), demonstrating transfection of Cannabis meristems using disarmed strains of both Agrobacterium rhizogenes (Ar18r12v) and Agrobacterium tumefaciens (GV3101).

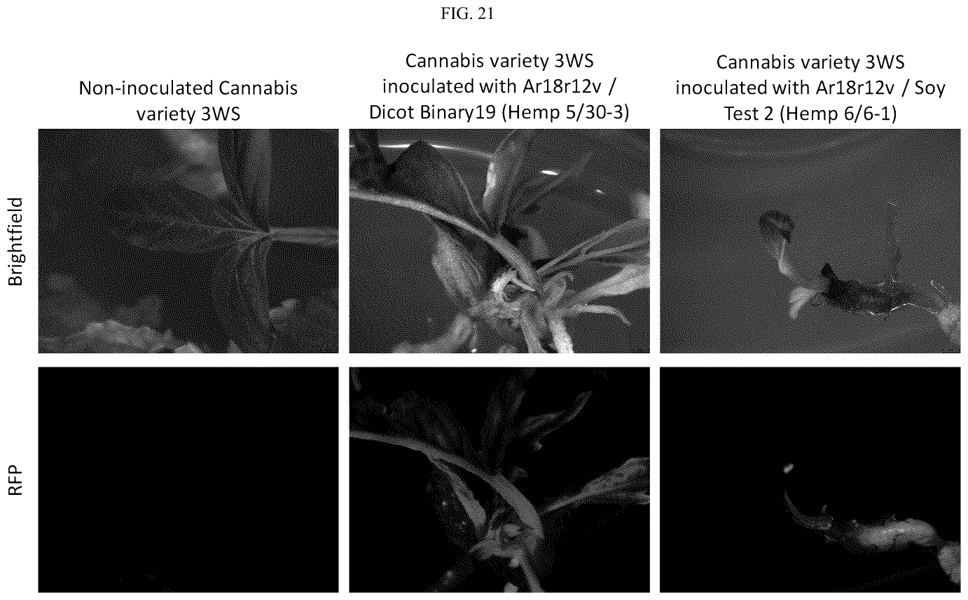

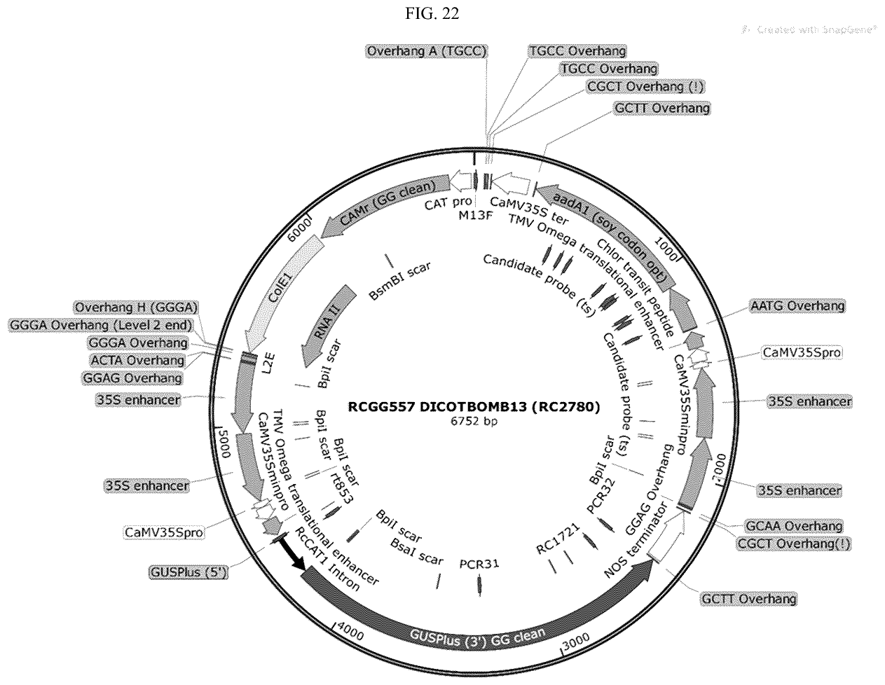

We have obtained additional stable RFP expressing Cannabis from experiments in the Honey Gold 3WS variety. The plantlet in the center of FIG. 21 (Cannabis plant WP421-1) is rooting on 50 mg/L streptomycin hemp node media (after being on 50 mg/L spectinomycin hemp node media for approximately 1 month).

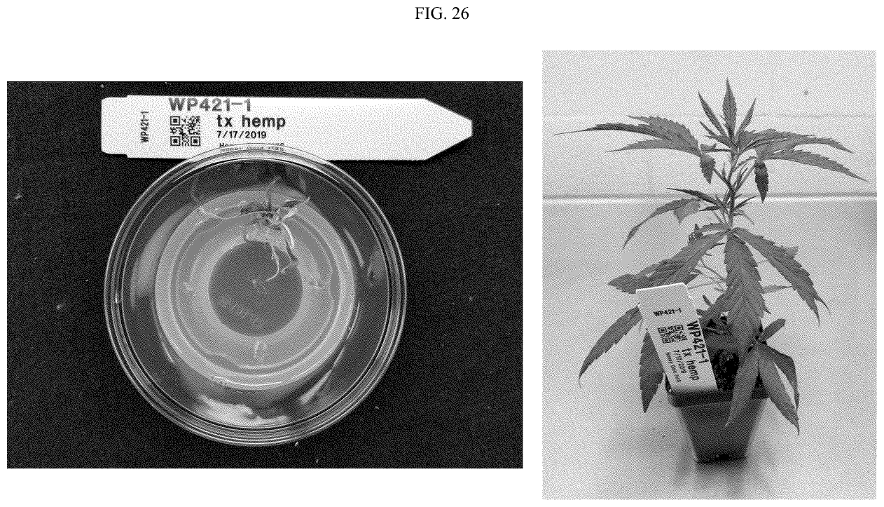

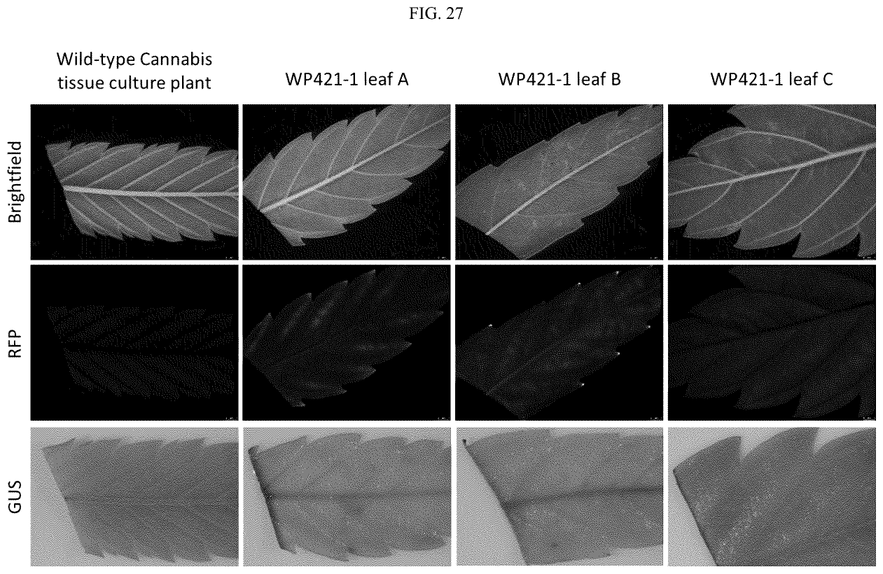

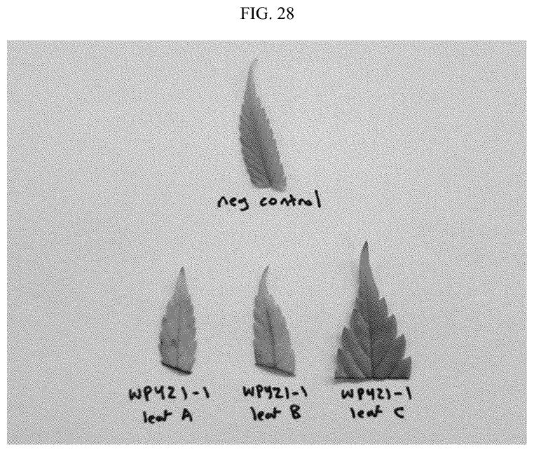

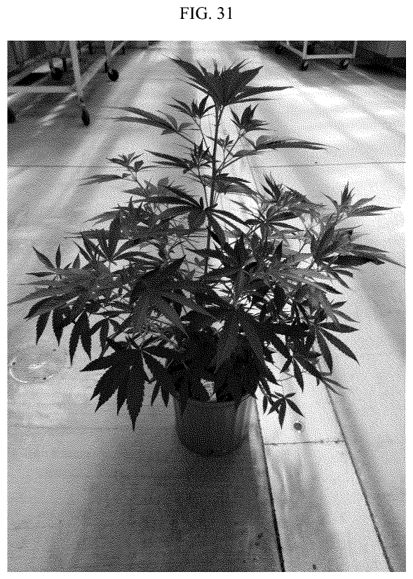

Plant WP421-1 was transferred to the greenhouse and imaged the day of transfer, approximately 4 weeks later (FIG. 26), and approximately 7 weeks later (FIG. 31). Three leaves from WP421-1 were sampled after it had been in greenhouse for 4 weeks. Two of the three leaves were stably expressing RFP (FIG. 27), while all three were stable expressing GUS (FIGS. 27 and 28). RFP expression was relatively weaker than when we initially sent the plant, which is likely due to tissue age. That all three randomly selected leaves are stably expressing GUS is a good indicator WP421-1 is not overly chimeric.

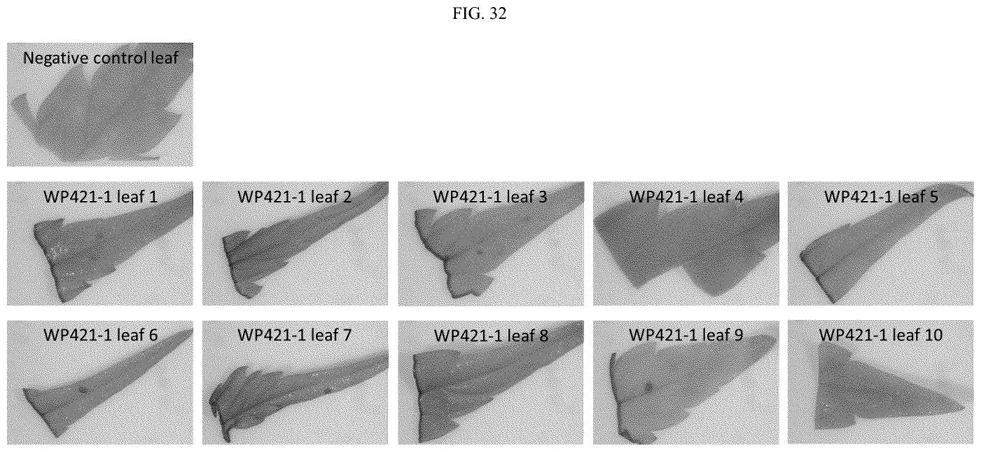

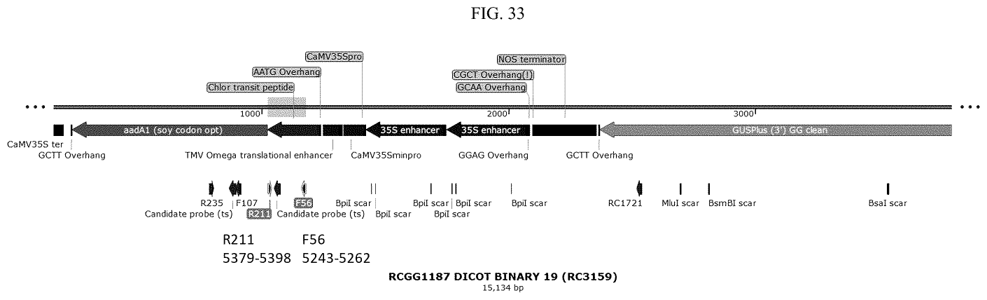

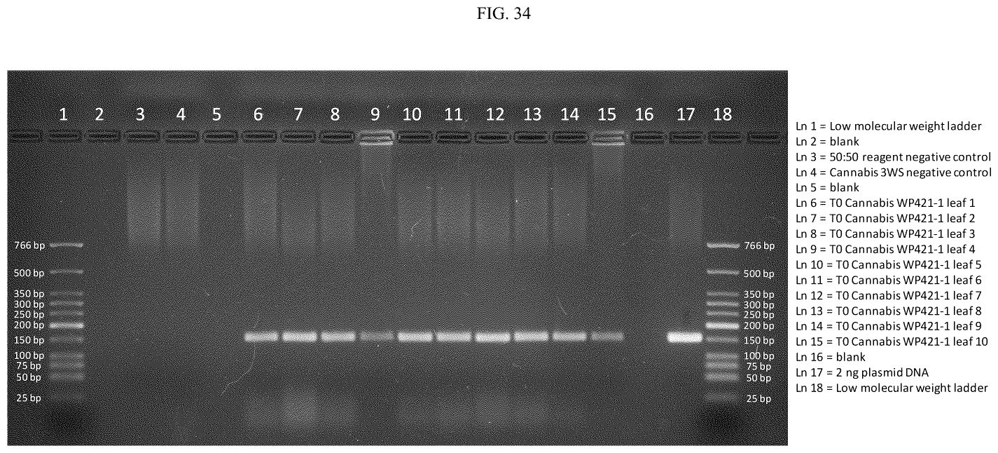

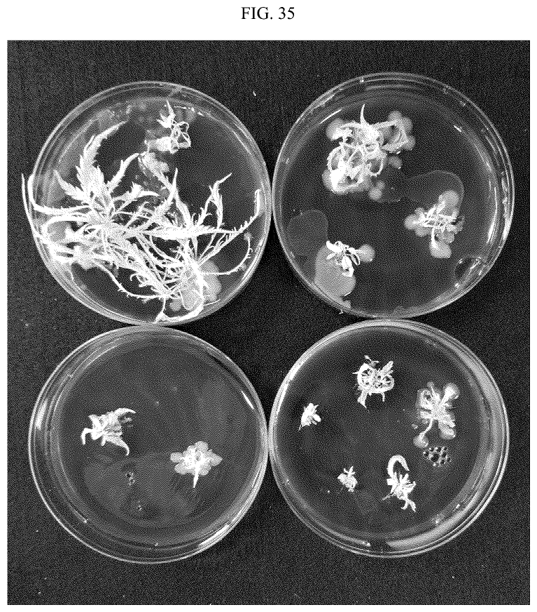

Additional data in FIGS. 32-34 confirms the chimerism in the WP421-1 plant. 10 leaf samples were taken from EP421-1 and divided for PCR (FIGS. 33-34) and GUS expression (FIG. 32) analysis. For PCR, we amplified a 156 bp fragment within the aadA expression cassette of DICOTBINARY-19 using primers designated F56 and R11 (fragments and amplicon highlighted in blue in FIG. 33). Leaf DNA from WP421-1 was extracted using the REDExtract-N-Amp™ Plant PCR Kit (Sigma-Aldrich XNAP-1KT) following manufacturer's instructions. PCR reaction was run with following:
1. 3 minutes at 94 C for initial denaturation
2. 30 seconds at 94 C for denaturation
3. 30 seconds at 55 C for annealing
4. 1 minute at 72 C for primer extension
5. Cycle steps 2-4 34 more times (35 total cycles)
6. 10 minutes at 72 C for final primer extension

PCR products were run on 1.5% agarose gel in SB buffer. All 10 leaf samples gave the expected 156 bp product, confirming minimal chimerism in this event

We have also blasted DNA into Cannabis meristem explants using PDS-1000 Helium gun with a plasmid designated DICOTBOMB-13. For particle bombardment experiments, gold-DNA “bead prep” was prepared by first washing 50 mg 0.6 um gold microcarriers (BioRad part #1652262) in 1 ml 100% ethanol and sonicating for 1 min 45 kHz. Gold was pelleted by centrifugation at 5000 rpm in microfuge (˜2300×g) and ethanol removed. Gold was then resuspended in 1 ml 100% ethanol and stored at-20 C until use. To precipitate DNA onto beads, the 50 mg gold/1 ml ethanol stock was sonicated for 1 min 45 kHz. 42 ul of this stock was transferred to an Eppendorf tube, then pelleted by centrifugation at 2500 rpm for 10 seconds, after which ethanol was removed. 500 uL sterile water was added and mixture sonicated 1 min 45 kHz. Gold was again pelleted by centrifugation at 2500 rpm for 10 seconds and water removed. 25 ul sterile water was then added, followed by sonication for 1 min 45 kHz. 2.6 ug DICOTBOMB-13 DNA was added, then sterile water to bring volume up to 245 ul. 250 ul cold 2.5 M CaCl₂ was added, followed by 50 ul 0.1 M spermidine. Solution was mixed by low speed vortexing. Tube was incubated on ice for approximately 1 hour with gentle inversions every 5-10 minutes. DNA/gold was pelleted at 1000 rpm (˜100×g) for 2 min and supernatant removed. Pellet was then washed with 1 ml 100% EtOH w/pipette tip, then pelleted again at 1000 rpm (˜100×g) for 2 min and supernatant removed 36 ul 100% EtOH was added to tube and gold completely resuspended with low-speed vortexing. Bead prep was stored at −20 C until used, with 5 ul used per bombardment. This corresponds to 360 ng DNA per blast; 290 ug gold per blast (1.2 ng DNA per ug gold).

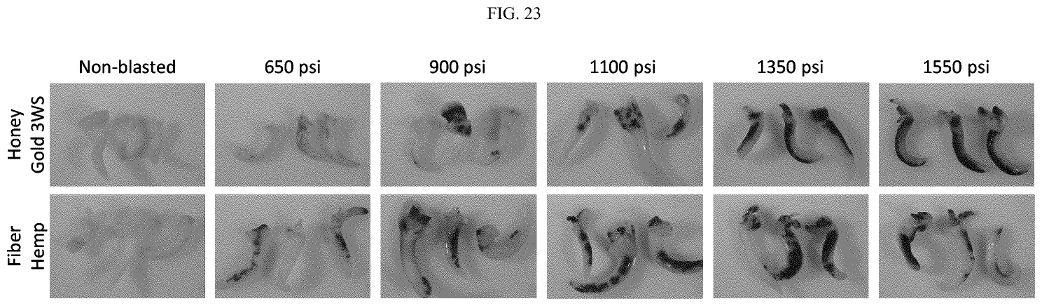

Cannabis meristem explants were excised from seed and precultured on EJW1 media overnight at 28 C 16/8 photoperiod, arranged on 12% xanthan gum targeting plates (with 60 mg/L Cleary's 3336 fungicide), blasted at 6 cm from the launch assembly using 5 uL bead prep per target and a range of rupture disks (650 psi-1550 psi), then allowed to rest on EJW1 media overnight. Transients were taken the next day. GUS transient expression in Cannabis meristem explants bombarded with DICOTBOMB-13 is shown in FIG. 23.

TABLE 10
Preculture Medium EJW1

| Ingredients and Notes | Amount to add per liter (grams) |
|---|---|
| MS salts no vitamins | 4.3 |
| Sucrose | 30 |
| 2,4-D (1 mg/ml stock) | 0.2 ml |
| MES | 2 |
| Cleary's 3336 | .03 |
| pH | 5.6 |
| Agarose | 4 |
| Autoclave | |
| Carbenicillin (100 mg/ml) | .25 |
| TDZ (1 mg/ml stock) | 1 ml |

### Example 2

The embodiments described herein demonstrate Cannabis floral dip experiments.

We used strain Ar18r12v based on positive GUS transients in meristem explants, harboring DICOTBINARY-22, which has the aadA1a protein targeted to both plastid and mitochondria in the plants. Agrobacterium cultures were resuspended in 5% sucrose with 0.05% silwet L-77 as a wetting agent. One of the cultures was induced with 100 uM acetosyringone and one was not. These cultures were applied directly to female flowers (some flowers received only 5% sucrose +0.05% silwet L-77 “blank”). Experiment is summarized in Table 11.

TABLE 11
Description and summary of experiments with Cannabis floral dip

| Experiment | | | Co-Culture Duration | Observations and |
|---|---|---|---|---|
| ID | Comments | AS | (Days) | Comments |
| Hrmp 4/17-1 | Agro washed once with | none | Agro applied directly to | 5 female plants; 3 |
| | sterile water; spun 10 | | flowers; 1 d dark in high | hermaphrodites |
| | min, resuspended in 5% | | humidity LEDA; then | (red/white twist tie |
| | sucrose; Silwet L-77 | | moved to GH7 with 3 | below flower for |
| | added to 0.05% | | males | inoculated, white for |
| | | | | blank) |
| Hemp 4/17-2 | Agro washed once with | 100 uM AS | Agro applied directly to | 5 female plants; 3 |
| | sterile water; spun 10 | | flowers; 1 d dark in high | hermaphrodites |
| | min, resuspended in 5% | | humidity LEDA; then | (green twist tie |
| | sucrose; Silwet L-77 | | moved to GH7 with 3 | below flower for |
| | added to 0.05% | | males | inoculated, white for |
| | | | | blank) |

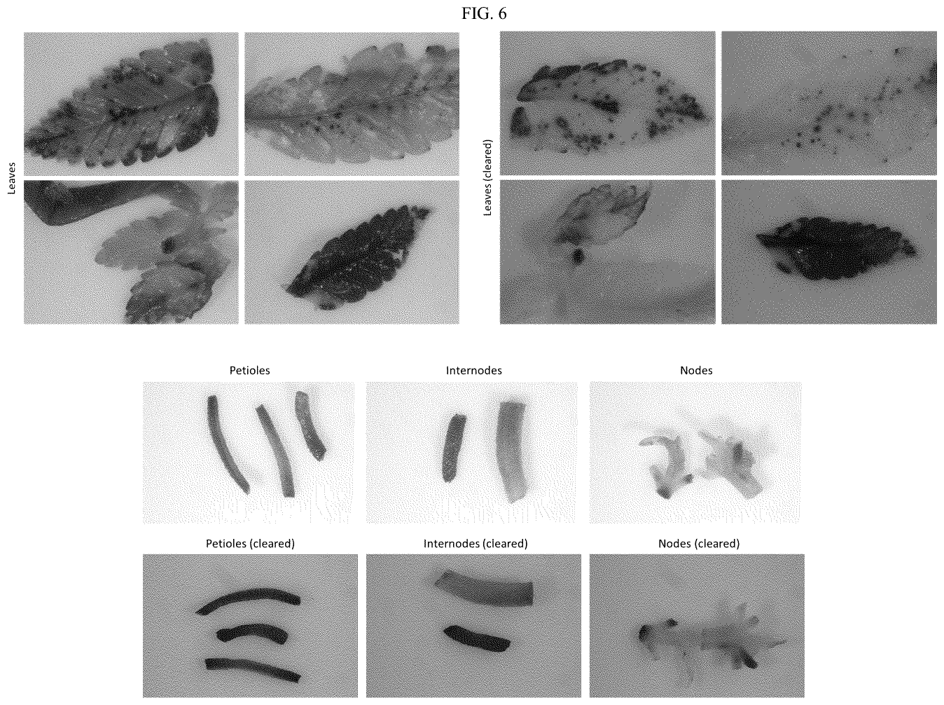

We noted no obvious ill effects of the inoculum application to the flowers one-day post-application. See FIG. 5. Female flowers were then pollinated with male pollen from donor plants by dusting the plants with pollen; as well as using a paint brush to apply pollen earlier.

### Example 3

The embodiments described herein demonstrate Cannabis node, internode, leaf, and petiole transformation experiments.

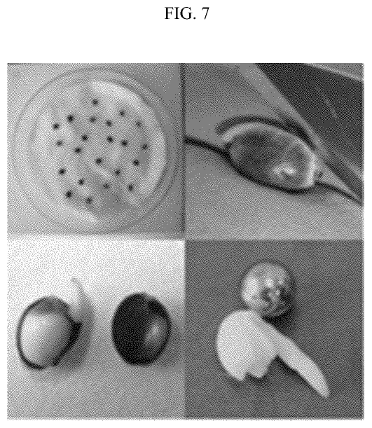

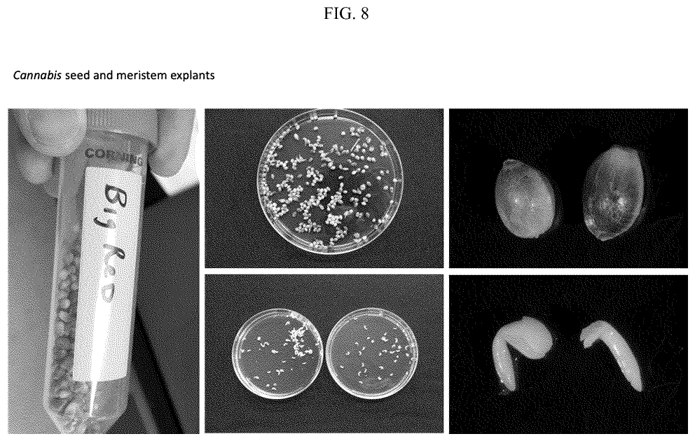

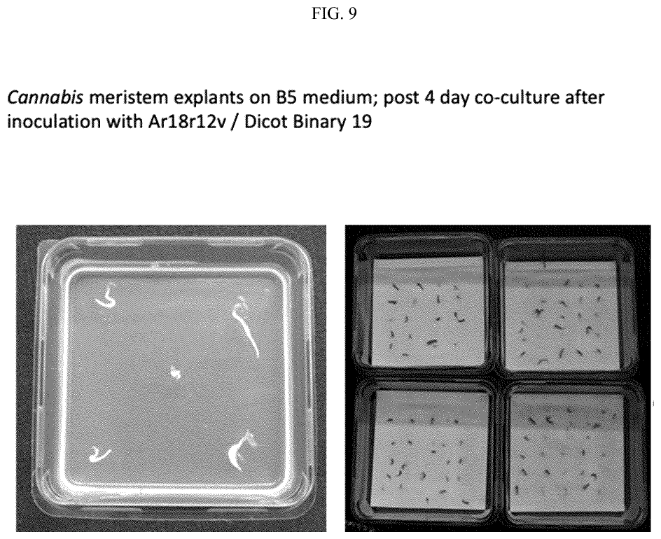

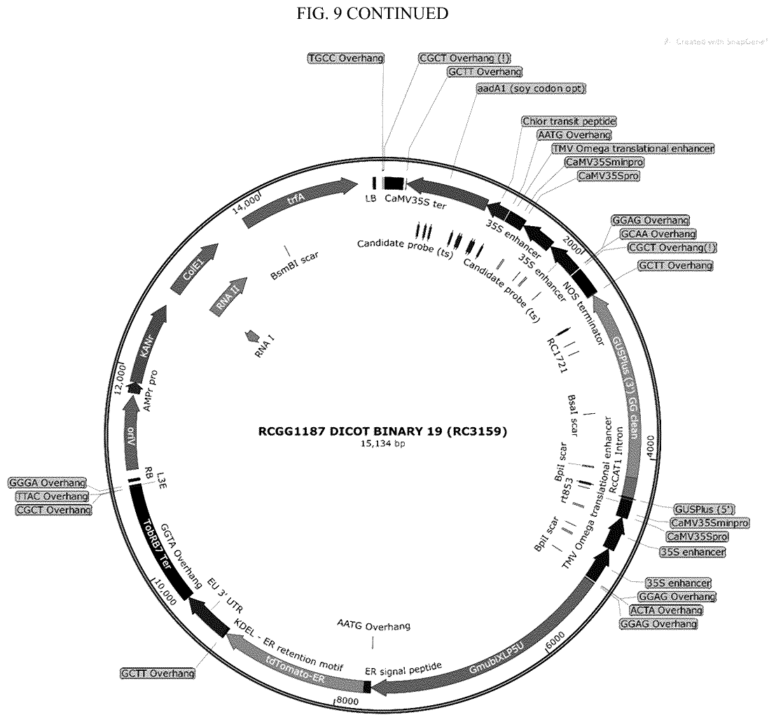

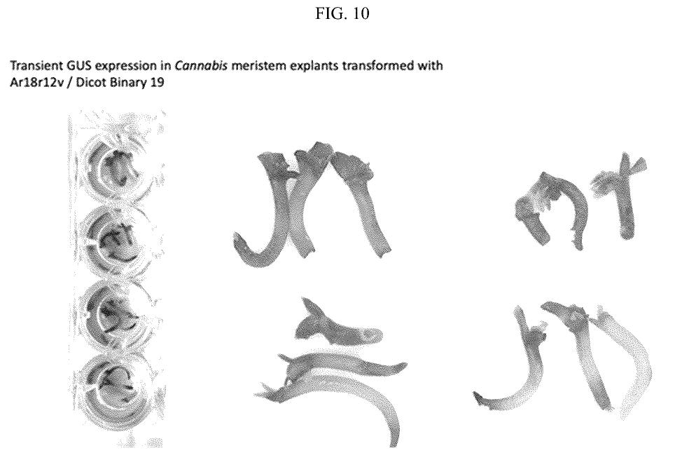

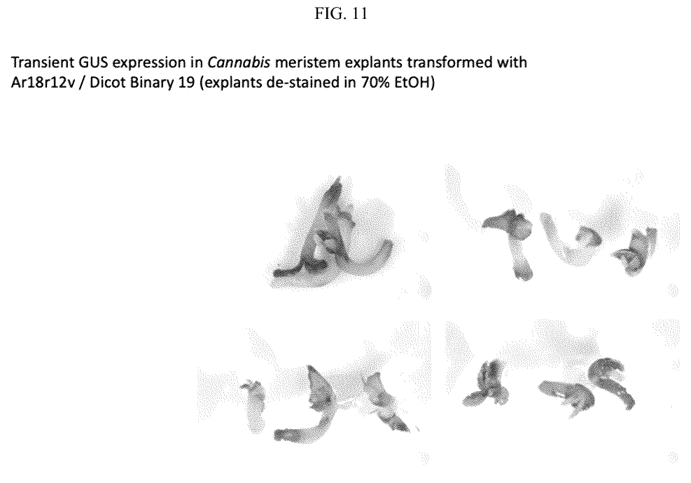

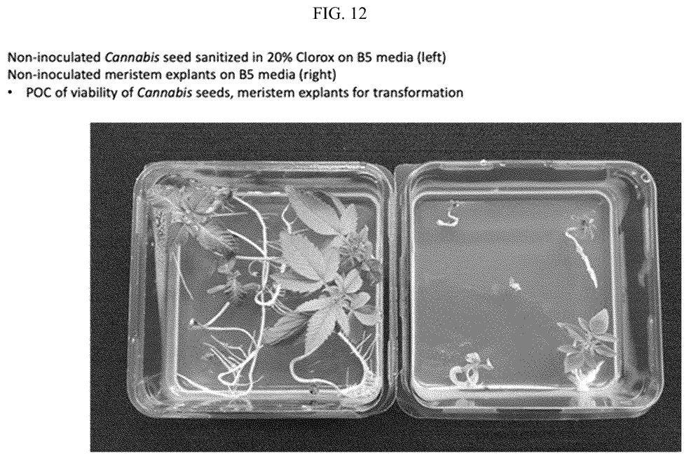

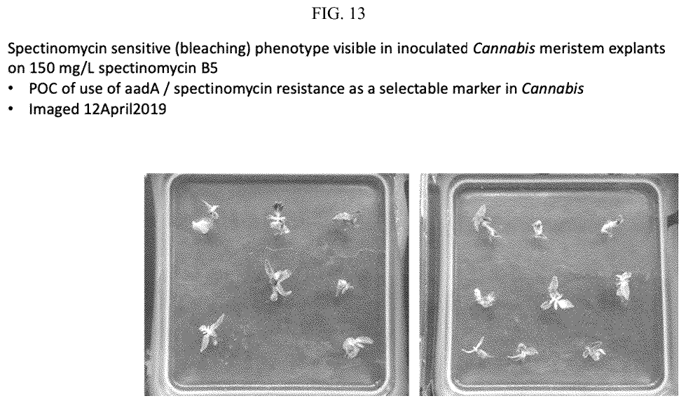

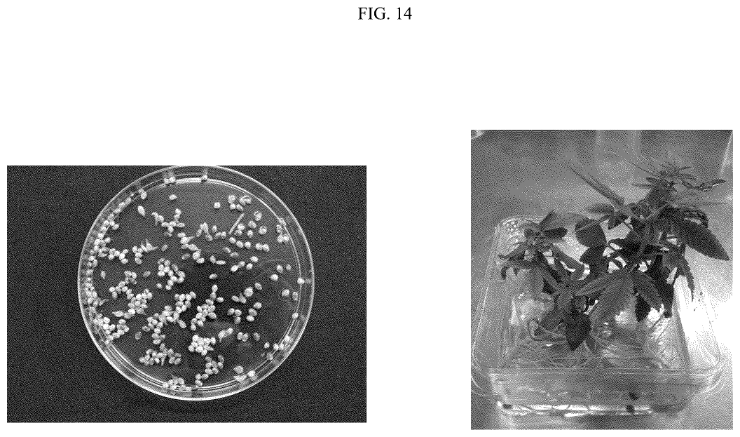

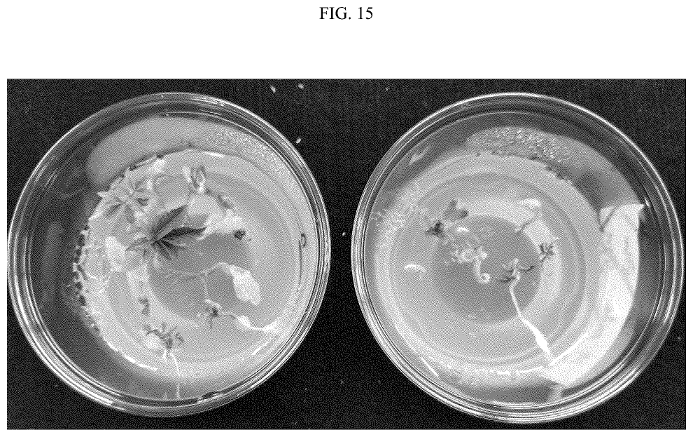

We inoculated node, internode, leaf, and petiole explants of Cannabis variety Elektra x Chardonnay. We used aseptically grown plantlets from sanitized and imbibed seed plated on non-selective B5 media for approximately 6 weeks. Explants were sonicated for 20 seconds at ˜45 kHz in the presence of Ar18r12v/DICOTBINARY-19. Nodal explants were co-cultured on 2.5 ml WCIC INO media with 50 mg/L nystatin, 10 mg/L TBZ, and 0.5 mg/L meta-topolin (mT). Internode, leaf, and petiole explants were co-cultured on 2.5 ml WCIC INO media with 50 mg/L nystatin, 10 mg/L TBZ, 1 mg/L meta-topolin (mT), 1 mg/L napthylacetic acid (NAA), and 0.2 mg/L GA3. After 4 days of co-culture at 23 C 16/8 photoperiod, GUS transient expression was observed in all explant types. See FIG. 6.

After co-culture, nodal explants were transferred to 100 mg/L spectinomycin hemp node media (Table 7) supplemented with 500 mg/L activated charcoal. Leaf, petiole, and internode explants were transferred to 100 mg/L spectinomycin hemp internode media, which is a modification of potato ZIG media (Cearley JA, Bolyard MG: Regeneration of Solanum tuberosum cv. Katahdin from leaf explants in vitro. Am Potato J 74:125-129 (1997)). A summary of these experiments is provided in Table 13.

TABLE 12
WCIC Hemp Internode Media (modified from Cearley 1997)

| Ingredients and Notes | Amount to add per liter (grams) |
|---|---|
| MS Salts complete with vitamins | 4.43 |
| (PhytoTech M519) | |
| Sucrose | 20 |
| Cleary's 3336 | 0.06 |
| pH to 5.7 with 1N KOH | |
| Gelrite (or Phytagel), then autoclave | 2 |
| Meta-topolin (mT) (1 mg/ml) | 1 ml |
| Napthylacetic acid (NAA) (1 mg/ml) | 1 ml |
| GA3 (FS) Sigma Prod G7645 | 0.2 ml |
| (1 mg/ml) | |
| Carbenicillin (200 mg/ml) | 1.25 ml |
| Cefotaxime (100 mg/ml) | 2 ml |
| Selection | as needed |

TABLE 13
Description and summary of experiments with Cannabis nodes, leaves, petioles, and internodes.

| | | # | | | |
|---|---|---|---|---|---|
| | | embryos | | # explants | |
| Experiment | | to | | to second | |
| ID | Comments | Selection | Notes | media | 2nd media |
| Hemp 5/2-1 | Nodes from hemp seed sanitized and imbibed; germinated | 11 | 100 ppm spec hemp | NA | NA |
| | on B5 3/22/19; harvested in INO; inoculated and sonicated | | node media; 28 C. | | |
| | 20 s 45 kHz; co-cultured in plantcons with filter paper + 2.5 | | 16/8/photoperiod | | |
| | ml INO with 50 ppm nystatin +10 ppm TBZ + .5 ppm | | | | |
| | meta-topolin (mT); 23 C. 16/8 photoperiod | 68 | 100 ppm spec hemp | 18 | non selective |
| Hemp 5/2-2 | Leaves from hemp seed sanitized and imbibed; germinated | | internode media; | | hemp node |
| | on B5 3/22/19; harvested in INO; inoculated and sonicated | | 28 C. | | media; 28 C. |
| | 20 s 45 kHz; co-cultured in plantcons with filter paper + 2.5 | | 16/8/photoperiod | | 16/8/photoperiod |
| | ml INO with 50 ppm nystatin + 10 ppm TBZ + 1 ppm | | | | |
| | meta-topolin (mT) + 1 ppm NAA + 0.2 ppm GA3; 23 C. | | | | |
| | 16/8 photoperiod | | | | |
| Hemp 5/2-2 | Petioles from hemp seed sanitized and imbibed; germinated | 68 | 100 ppm spec hemp | 3 | non selective |
| | on B5 3/22/19; harvested in INO; inoculated and sonicated | | internode media; | | hemp node |
| | 20 s 45 kHz; co-cultured in plantcons with filter paper + 2.5 | | 28 C. | | media; 28 C. |
| | ml INO with 50 ppm nystatin + 10 ppm TBZ + 1 ppm | | 16/8/photoperiod | | 16/8/photoperiod |
| | meta-topolin (mT) + 1 ppm NAA + 0.2 ppm GA3; 23 C. | | | | |
| | 16/8 photoperiod | | | | |
| Hemp 5/2-2 | Internodes from hemp seed sanitized and imbibed; | 68 | 100 ppm spec hemp | 3 | non selective |
| | germinated on B5 3/22/19; harvested in INO; inoculated | | internode media; | | hemp node |
| | and sonicated 20 s 45 kHz; co-cultured in plantcons with | | 28 C. | | media; 28 C. |
| | filter paper + 2.5 ml INO with 50 ppm nystatin + 10 ppm | | 16/8/photoperiod | | 16/8/photoperiod |
| | TBZ + 1 ppm meta-topolin (mT) + 1 ppm NAA + 0.2 ppm | | | | |
| | GA3; 23 C. 16/8 photoperiod | | | | |

### Example 4

Cannabis meristem explants were excised, dried, and stored at −20 C. Cannabis meristem explants of variety 3WS were excised from seed and then dried on the surface of filter paper in a laminar flow hood for 26 hours. Dried Cannabis meristem explants were then stored at −20 C for 3 days. Explants were then rehydrated in 20% PEG4000 with 60 mg/L Captan and 30 mg/L Bravo fungicides, rinsed, and plated on Hemp Node media without activated charcoal (Table 7).

### Example 5

This embodiment describes the transformation of Cannabis pollen, in particular the advantages provided when pollen from male plants can be stored. Cannabis pollen was harvested in by shaking branches of male plants onto a creased sheet of blue paper (high visibility of yellow on blue). Pollen was poured through a #80 sieve into a 50ml conical tube. A small, visible amount of pollen was added to 7 1.5 mL microfuge tubes comprising the following treatments:
1. 25% glycerol (w/v) PGM, inverted several times to mix and stored at −20 C.
2. 25% glycerol (w/v) PGM, inverted several times to mix and stored at 4 C
3. Untreated, fresh pollen, stored at 4 C
4. Untreated pollen, stored at −20 C
5. Pollen placed in 1.5mL microfuge tube, placed in sweater box with MgNO3 solution. Moved to −20 C on 5/3 at 11 am.
6. 25% w/g glycerol PGM, 1 hour incubation at RT, followed by storage at −80 C
7. One hour untreated at room temperature in 50 mL tube.

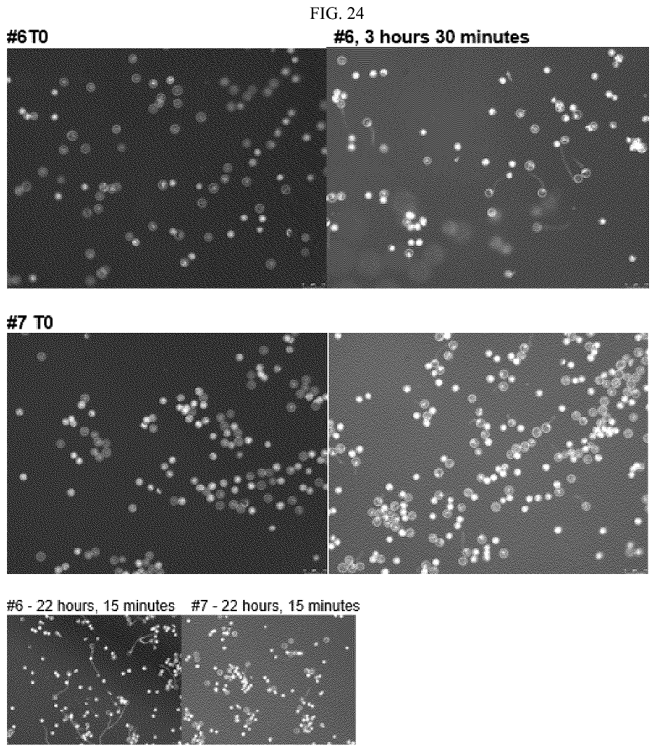

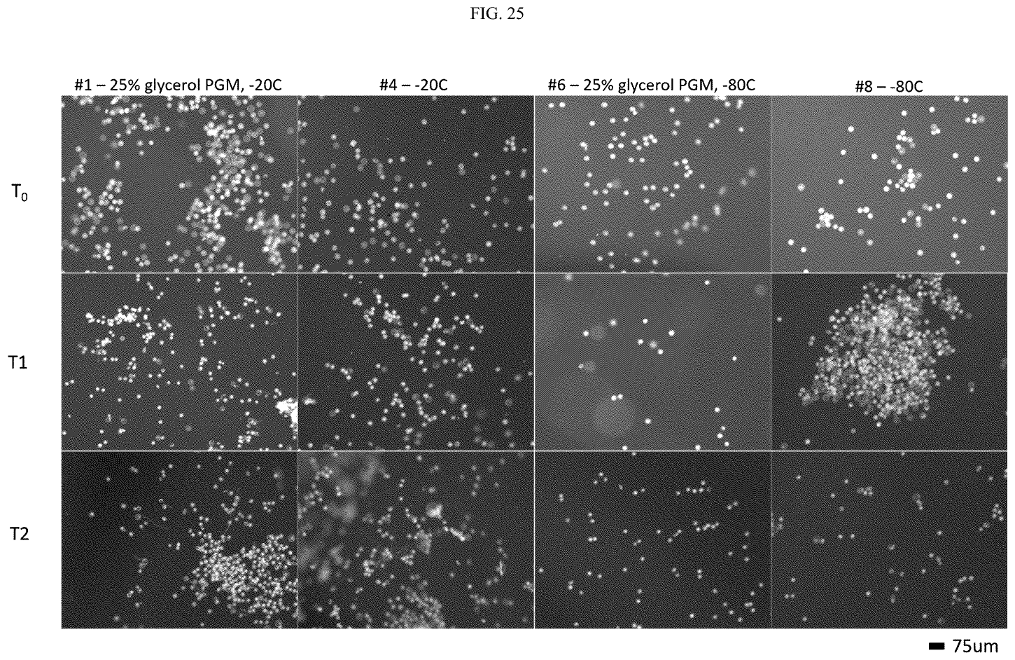

The same day pollen was harvested, pollen from tubes #6,7 were placed onto solid PGM after one hour of incubation at room temperature. Plates were then wrapped in parafilm and placed in dark room and examined under microscope for germination in the form of pollen tube expansion. Germination of tubes was visible on both. Tube emerged more quickly from pollen from tube #6, which may be due to the liquid PGM matrix. Germination continue onto the next day, 22 hours after plating. See FIG. 24.

To test for overnight storage, we assayed pollen for germination the next day. Pollen from tubes 1,4 and 6 were placed on ice to thaw. After half an hour, a small amount of pollen from each was added to a PGM plate partition. 50uL of pollen was taken from 1 & 6. Tube 6 was noticeably more opaque than tube 1. Tube germination was visible only in treatment #1. Multiple, branched tube-like structures were visible in treatment #4 at T2 (overnight storage) which may have been aberrant germination or the first indications of fungal hyphae. Following 24 H of incubation, tube germination was visible in the −20° C. stored pollen that was stored in 25% (w/v) glycerol PGM. No survival was observed in either −80° C. treatment, which perhaps indicates that a slower rate of freezing is important for retaining viability. See FIG. 35.

TABLE 14
Pollen Germination Medium - Salts II Solution (PGM2 Salts)
Pollen Germination Medium Salts II Solution (PGM2 Salts)

| | Volume: | | | |
|---|---|---|---|---|
| Stock (100X) | 10 mL | | Original | 100 mL stock |
| Reagent | CAS | g/10 mL | Units | grams added |
| Boric Acid (H3BO3) | 0043-35-3 | 0.05 | 0.005% | 0.5 |
| CaCl2 * 2H2O | 10035-04-8 | 1.47 | 10 mM | 14.7 |
| Potassium Phosphate, | 7778-77-0 | 0.00680 | 0.05 mM | 0.068 |
| monobasic (KH2PO4) | | | | |
| ddH2O (dissolve salts) | | Add 8 mL | | Add 80 mL |
| dissolve by stirring and/or | | | | |
| sonication | | | | |
| ddH2O | | Fill to 10 mL w/ | | Fill to 100 mL w/ |
| | | graduated cylinder | | graduated cylinder |

TABLE 15
2X PGM (Pollen Germination Medium)
2X Pollen Germination Medium

| | 1 L | |
|---|---|---|
| Volume | amount | units |
| PGM Salts | 10 | mL |
| Sucrose | 100 | g |
| PEG 4000 | 600 | g |

| | |
|---|---|
| ddH2O | Fill to 1 L |
| Heat to 70 C. for 10 minutes on stir plate. | |
| Filter sterilize. | |
| Store at 4 C. | |

TABLE 16
Solid Pollen Germination Medium
Solid Pollen Germination Medium

| | 1 L | |
|---|---|---|
| Volume | amount | units |
| 2X PGM | 500 | mL |
| 0.6% Noble Agar | 500 | mL |

TABLE 17
25% glycerol PGM
25% glycerol PGM

| | 1 L | |
|---|---|---|
| Volume | amount | units |
| 2X PGM | 500 | mL |
| 50% (w/y) glycerol | 500 | mL |

### Example 6

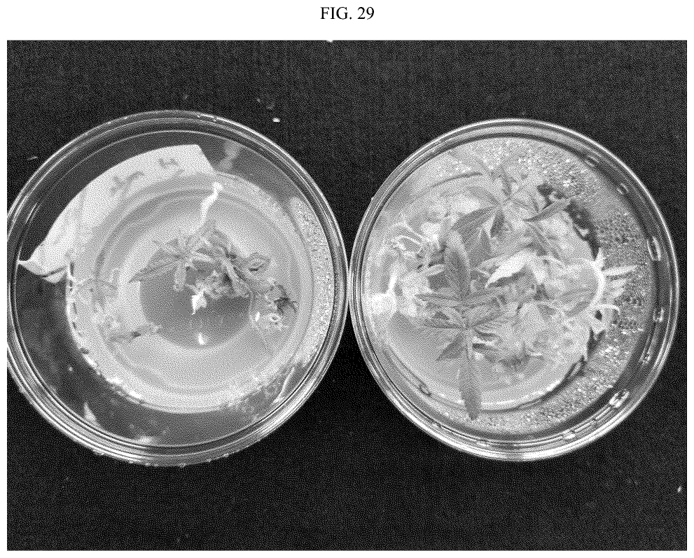

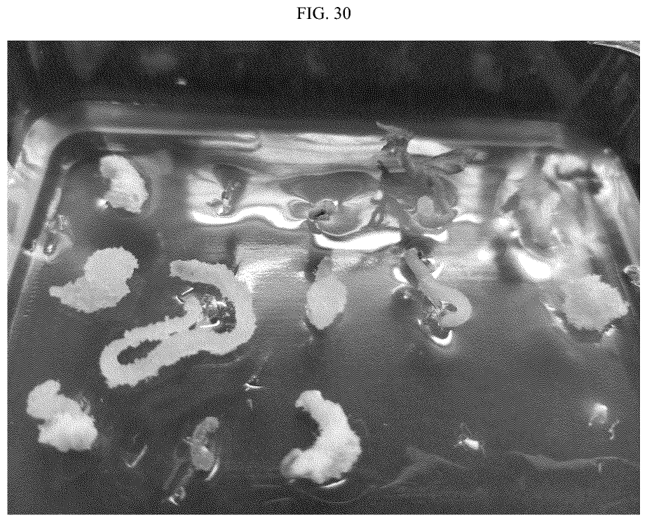

Cannabis value added explants (VAEs) were generated by hand excising meristem tissue from seeds surface sanitized in 20% Clorox for 5 min, rinsed, and imbibed for ˜20 h in BGM. Cannabis VAEs of variety 3WS were excised from seed, then dried on the surface of a filter paper in a laminar flow hood for 26 hours. Dried Cannabis meristem explants were then stored at −20 C for 3 days. Explants were then rehydrated in 20% PEG4000 with 60 mg/L Captan and 30 mg/L Bravo fungicides, rinsed, and plated on Hemp Node media without activated charcoal. FIG. 29 shows dried Cannabis VAEs after 5 weeks plated on non-selective medium. FIG. 30 shows machine excised Cannabis meristem explants on non-selective medium for 2 weeks.

Cannabis VAEs may also be primed with WCIC INO or other medium prior to drying. Examples of priming and drying conditions are outlined below. These examples are for soybean transformations but are applicable to Cannabis VAE priming and drying and the same or similar conditions may be used with Cannabis explants.

TABLE 18
Priming and Drying of Explants
(Soybean genotype W28, strain GV3101)

| | | | | T0 | |
|---|---|---|---|---|---|
| | | | Shoots | plants | |
| | Co- | Ex- | har- | to | |
| VAE Batch/Priming | culture | plants | vested | GH | TF |
| Fresh explants giving rise | 1 ppm | 75 | 6 | 3 | 4.0% |
| to S92 series | TDZ | | | | |
| Fresh explants giving rise | no TDZ | 71 | 3 | 3 | 4.2% |
| to S92 series | | | | | |
| S92A (std dry) | 1 ppm | 75 | 8 | 4 | 5.3% |
| | TDZ | | | | |
| S92B (3 hr prime in INO, | 1 ppm | 50 | 10 | 10 | 20.0% |
| then dry) | TDZ | | | | |
| S92C (3 hr prime in INO | 1 ppm | 75 | 9 | 6 | 8.0% |
| with 1 ppm | TDZ | | | | |
| TDZ, then dry) | | | | | |
| S92D (3 hr prime in INO | 1 ppm | 50 | 0 | 0 | 0.0% |
| with 10 ppm | TDZ | | | | |
| TDZ, then dry) | | | | | |
| S92E (3 hr prime in INO | 1 ppm | 63 | 0 | 0 | 0.0% |
| with 50 ppm | TDZ | | | | |
| TDZ, then dry) | | | | | |
| S92E (3 hr prime in INO | 1 ppm | 50 | 0 | 0 | 0.0% |
| with 100 ppm | TDZ | | | | |
| TDZ, then dry) | | | | | |

### Example 7

This example demonstrates additional embodiments of the transformation methods and transgenic Cannabis plants described herein.

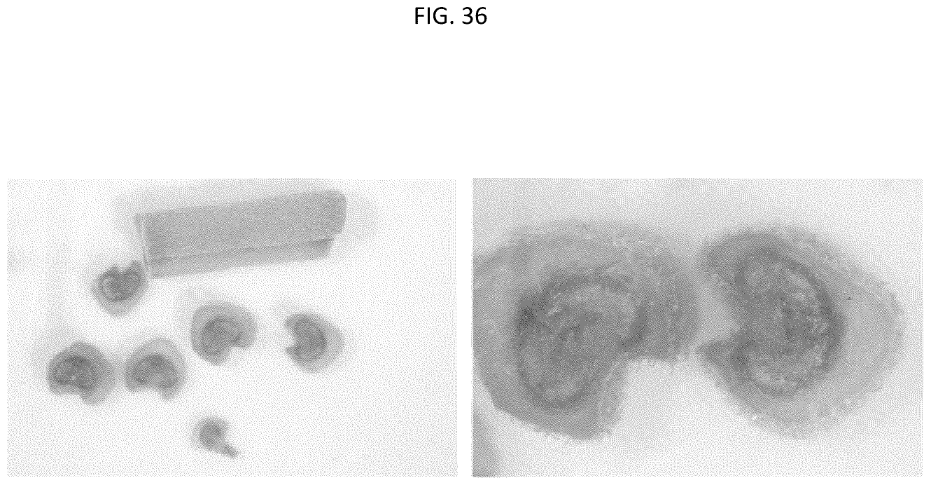

T1 data/POC of germline transformation of Cannabis using meristem explants: A leaf sample of Cannabis T0 event WP421-1 (plant “Candice”) was taken for long-term storage at −80 for further genetic analysis, and at this time we took sections of the petiole as well for GUS expression analysis. FIG. 36 shows GUS expression in the vascular tissue of WP421-1, which is an indicator of germline status for the transgene.

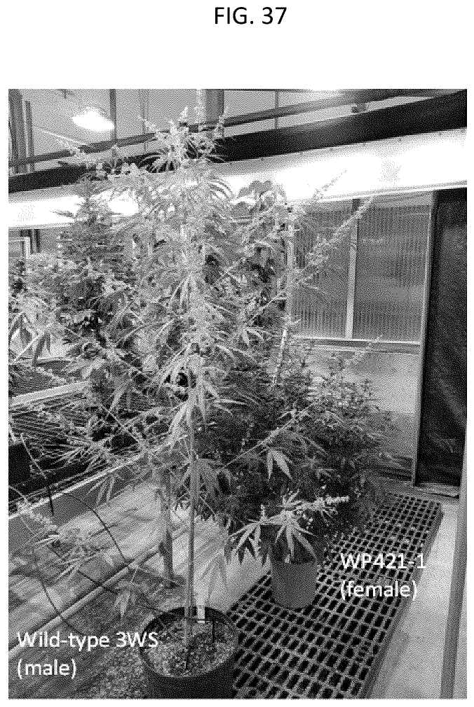

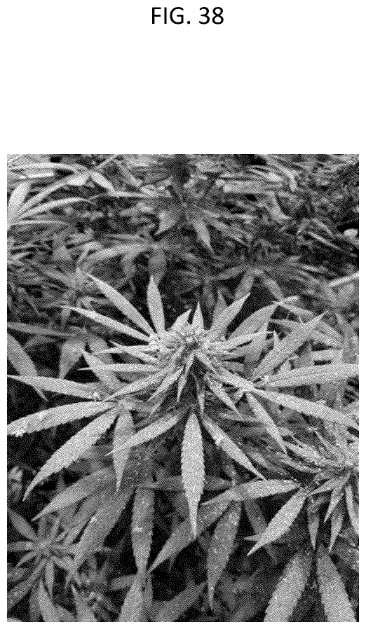

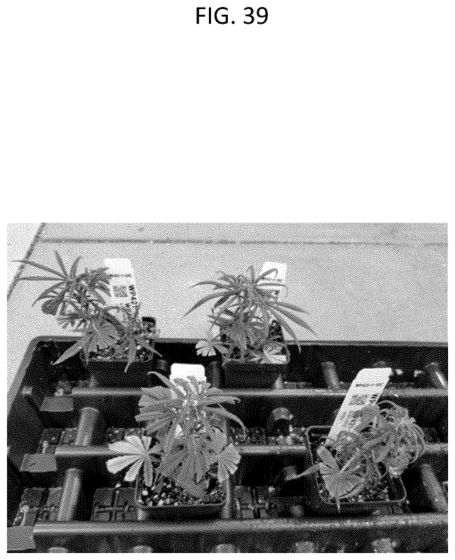

WP421-1 was moved to short days in GH15 approximately 9 weeks after plant handoff to greenhouse. 10 cuttings were taken 2 days later. Some cuttings had roots approximately 2 weeks after cuttings were taken. First flowers (female) observed 5 days after moving plant to short days. WP421-1 was move into GH9 2 weeks after being put in short-day to be exposed to pollen from the 3WS 2-5 auto-flowering male. FIG. 37 shows WP421-1 adjacent to the male pollen donor wild-type 3WS plant.

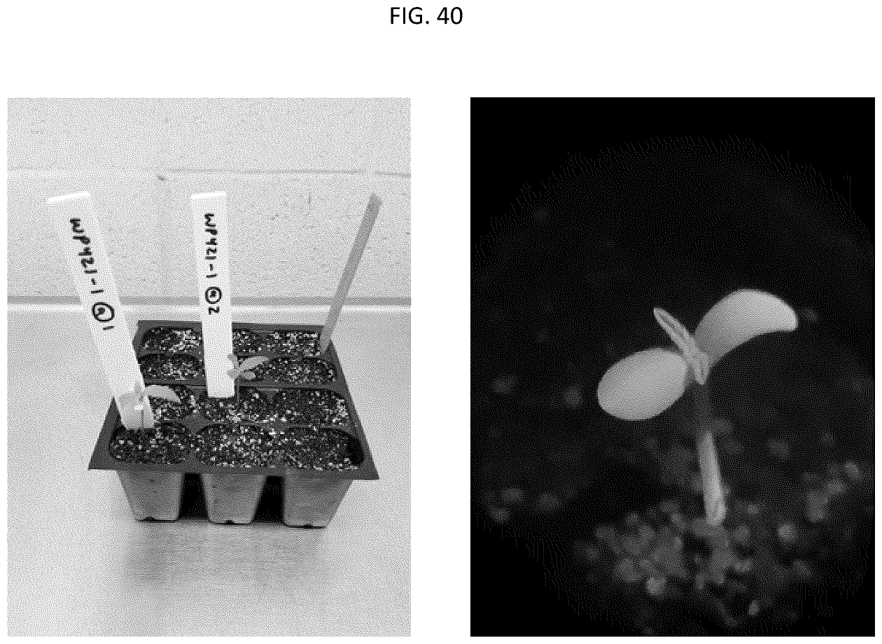

2.5 weeks after pollination with male 3WS plant, immature T1 seed of WP421-1 was planted. We noted RFP+expression (tdTomato) in one of the seedlings 3.5 weeks after this (in plantlet “Carly”), confirming germline transmission of transgene. (FIG. 40)

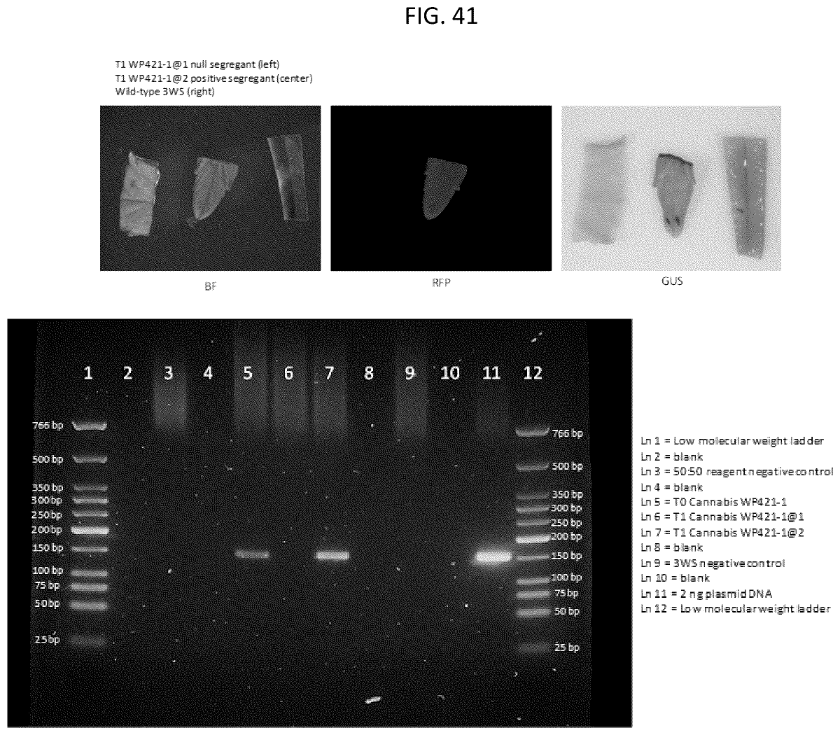

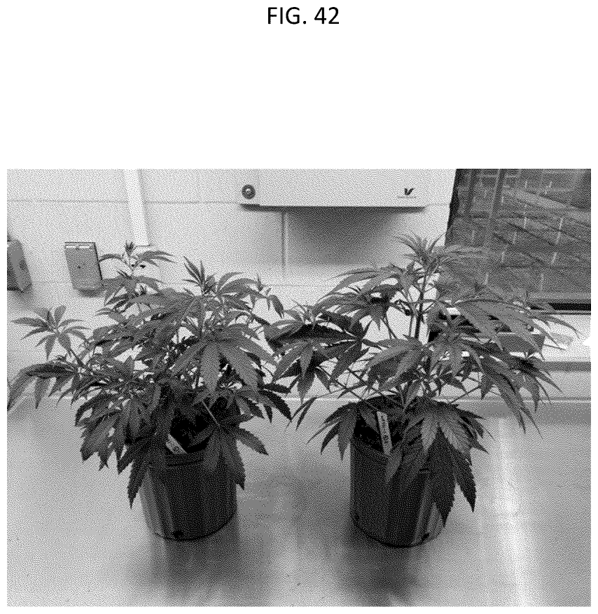

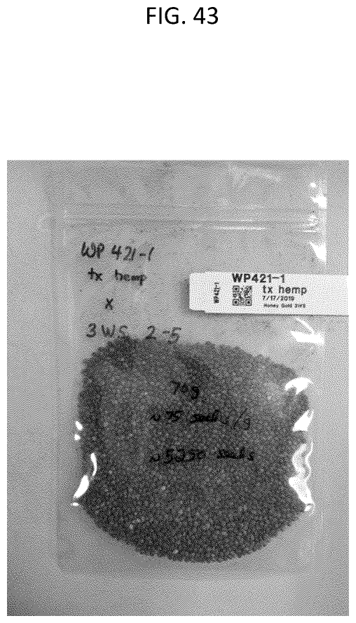

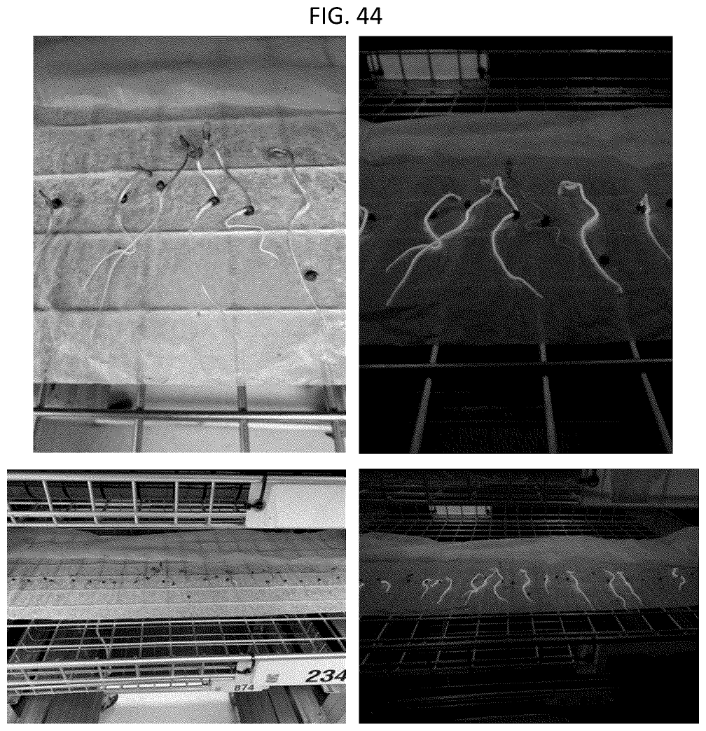

We followed this observation up with additional RFP expression (tdTomato) observations (eliminating background in a null segregant and in a wild-type leaf), GUS expression, and an aadA1a PCR that produced the expected 156 bp amplicon. (FIGS. 41 and 42). Approximately 5250 T1 seed were harvested from WP421-1 approximately 2 months after pollination was initiated (FIG. 43). We germinated T1 seed of WP421-1 on germination paper and planted a flat of T1 seed to examine more plants for RFP expression (tdTomato) and segregation. RFP expression (tdTomato) was apparent along with null segregants (FIG. 44)

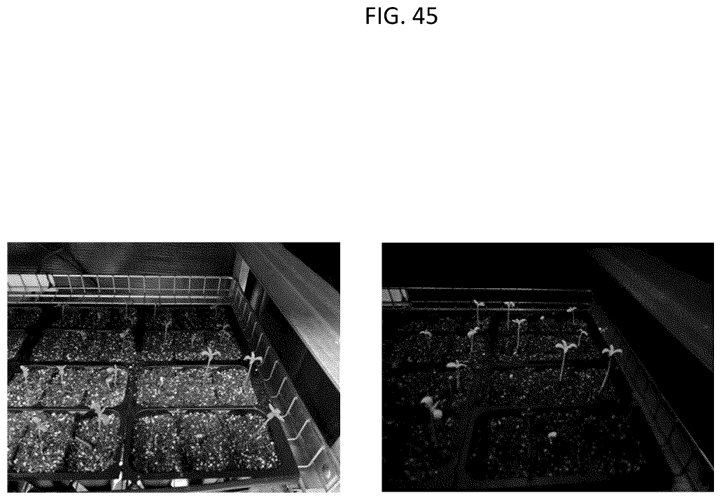

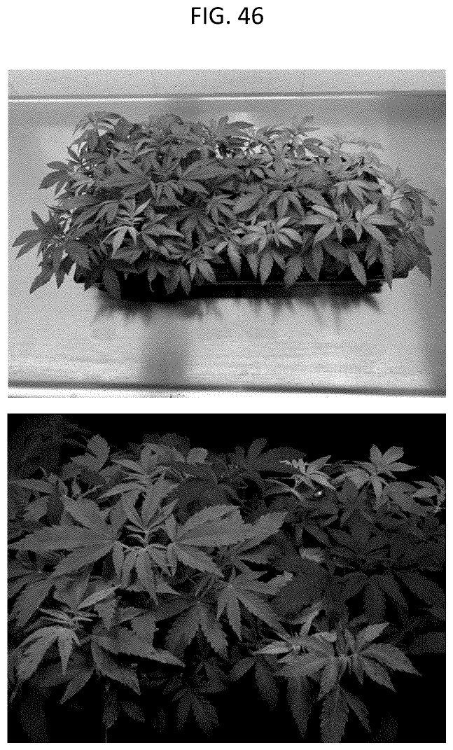

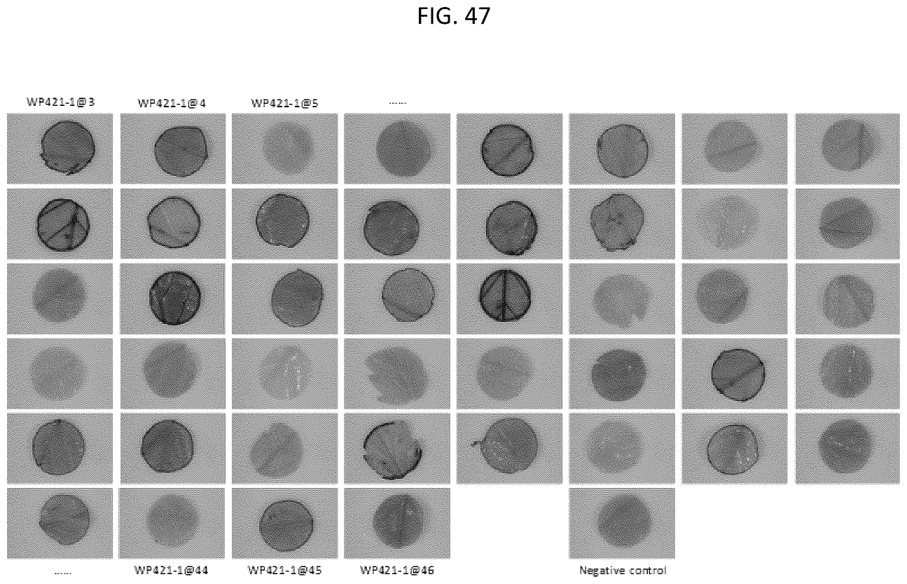

Of the 48 T1 WP421-1 seed planted in flats, 44 germinated and were designated WP421-1@3 through WP421-1@46. 22 of these 44 expressed RFP (tdTomato) and GUS, giving the expected 1:1 segregation ratio for this cross. (FIG. 45)

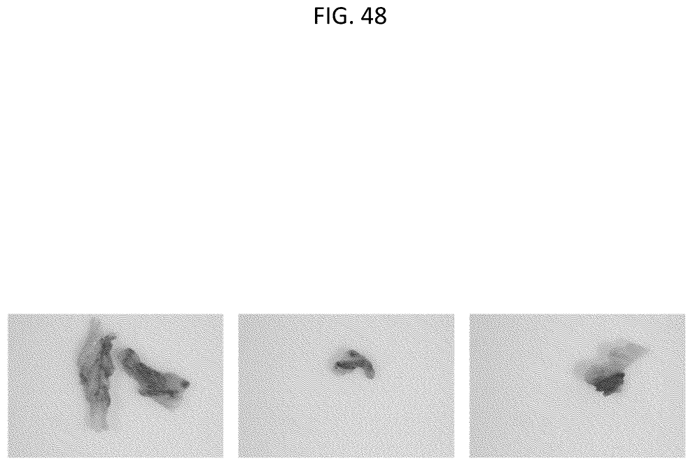

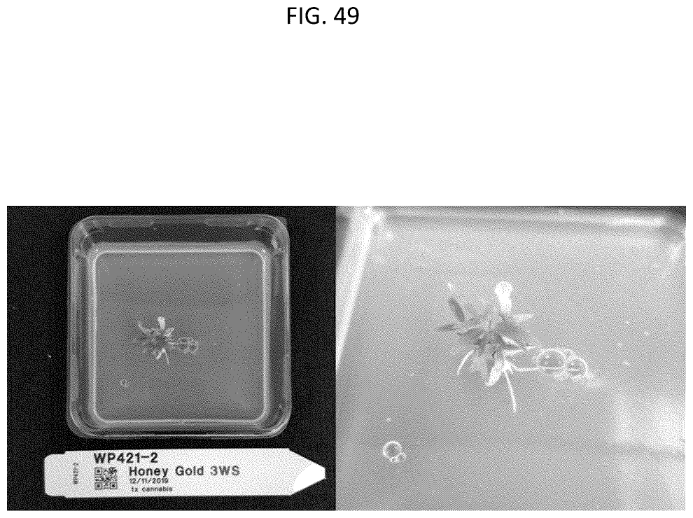

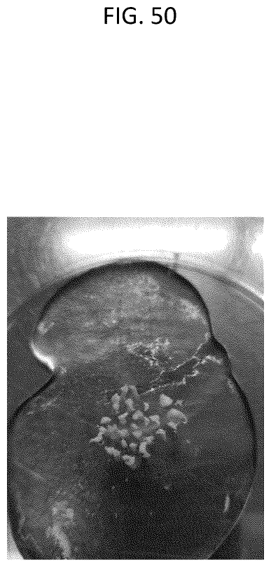

Additional Cannabis transformation experiments using meristem explants: Additional experiments with Cannabis meristem explants inoculated with Ar18r12v/DICOTBINARY-19 using 75 mg/L spectinomycin selection have yielded plantlets chimeric for GUS as well as greening plantlets that are GUS negative. These chimeric plantlets did not root on the subsequent 50 mg/L streptomycin media and were instead transferred to selection-free BRM (FIG. 48). One chimeric GUS+ plant (center photo in FIG. 48) from this set rooted on selection-free BRM and was sent to the greenhouse as the T0 plant WP421-2, but we could not detect positive GUS or RFP expression or transgene integration by PCR in subsequent tests of WP421-2, possibly indicating negative sectors of the plant outcompeted transgenic sector (FIG. 49).

We have regenerated a transgenic Cannabis plantlet from particle-mediated transformation that is stably expressing GUS in all it leaves. Cannabis explants prepared from seed germinated at 37 C overnight of variety 3WS were precultured on EJW1 overnight and blasted on carboxymethylcellulose media using DICOTBOMB-13 (at 1.2 ng DNA/ug gold; 0.6 um gold; 1 cm gap, 6 cm distance, and 1350 psi).

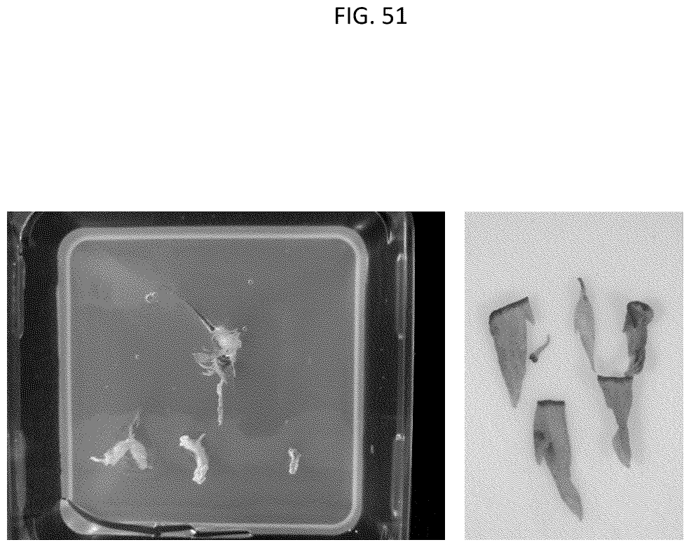

Explants were rested on EJW1 overnight, then transferred to 100 mg/L spectinomycin hemp node media without activated charcoal for 1 month. Explants were then transferred to 50 mg/L streptomycin hemp regeneration media with 1 mg/L meta-topolin (mT). Leaves were sampled from greening explant and incubated in X-gluc at 37 C. Stable GUS expression was present in every leaf, demonstrating POC of particle-mediated transformation of Cannabis meristem explants (FIG. 51).

### Example 8

Transgenic T0 Cannabis plant generation using particle-mediated transformation of Cannabis meristem explants

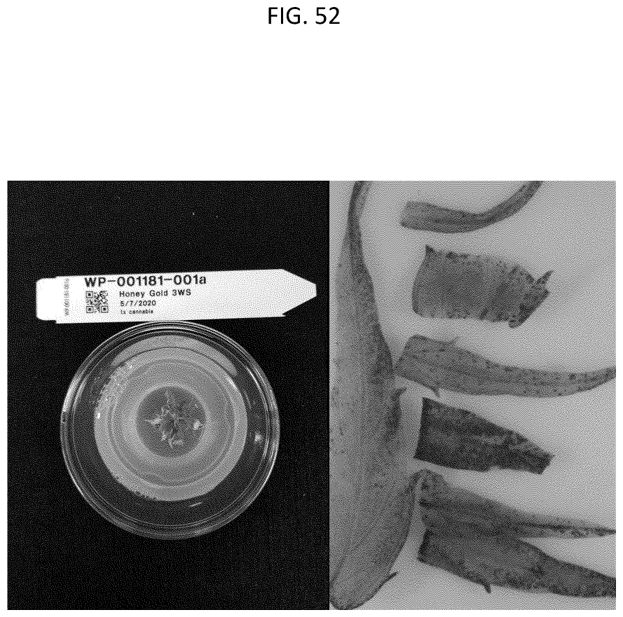

The regenerating transgenic Cannabis plantlet from particle-mediated transformation stably expressing GUS in all it leaves from last report has rooted and been sent to GH as T0 plant WP-001181-1. This T0 plant was derived from Cannabis meristem explants prepared from seed germinated at 37 C overnight of variety 3WS. Meristem explants were precultured on EJW1 overnight and blasted on carboxymethylcellulose media using DICOTBOMB-13 (at 1.2 ng DNA/ug gold; 0.6 um gold; 1 cm gap, 6 cm distance, and 1350 psi). Explants were rested on EJW1 overnight, then transferred to 100 mg/L spectinomycin hemp node media without activated charcoal for 1 month (media previously described). Explants were then transferred to 50 mg/L streptomycin hemp regeneration media with 1 mg/L meta-topolin (mT) for 6 weeks (media previously described). We then attempted to root this explant by transferring it to: (i) 4 weeks on 50 mg/L streptomycin hemp node media after cutting hypocotyl to remove necrotic tissue; (ii) 2 weeks on non-selective B5 media; (iii) 6 weeks on non-selective dicot BRM media (1/2 MS salts with 0.1 mg/L IAA); (iv) Explant split into two; hypocotyl cut on each piece to remove necrotic tissue; and (v); 4 weeks (plant “A”) to 7 weeks (plant “B”) on non-selective dicot BRM media. We re-assayed leaves for GUS and found all leaves remained GUS+. (FIG. 52).

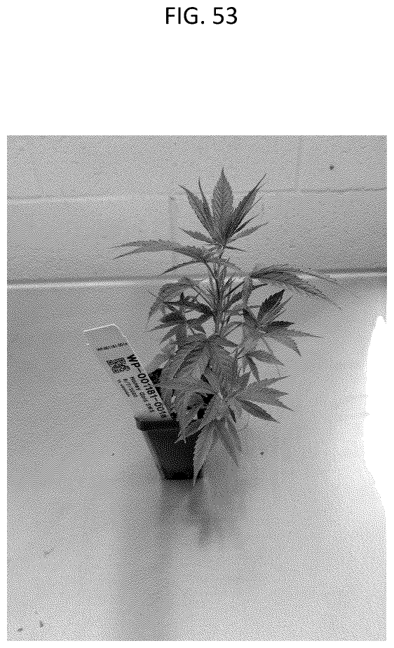

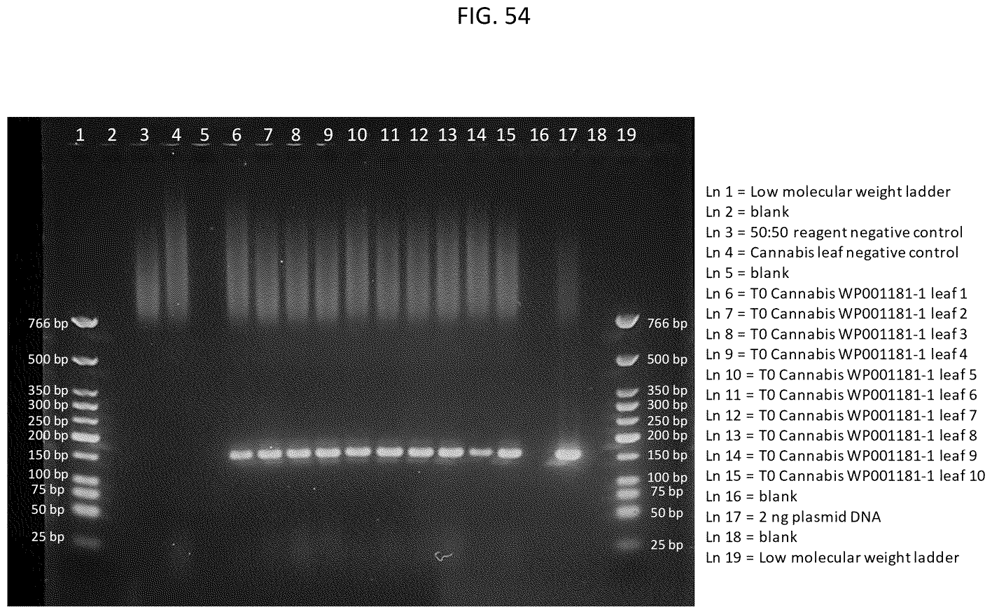

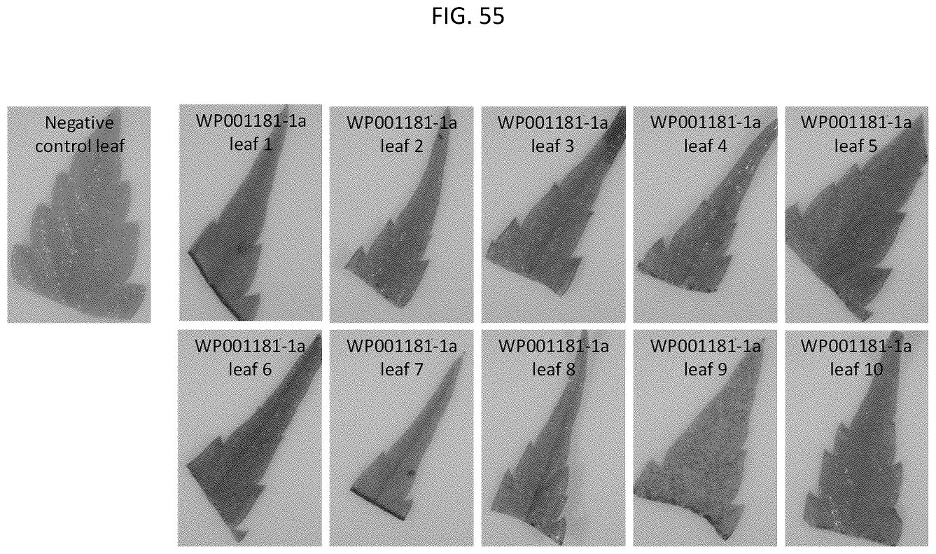

After the WP-00118la event had been in the greenhouse for 3 weeks, we used the previously described primer set and PCR conditions to amplify a 156 bp fragment of the aadA1a gene from 10 separate leaves. At the same time, we assayed these leaves for GUS expression (each leaf sample was divided in 2, one sample for PCR, one for GUS assay). All 10 leaves tested positive for aadA1a by PCR, and all 10 were GUS positive (FIG. 53).

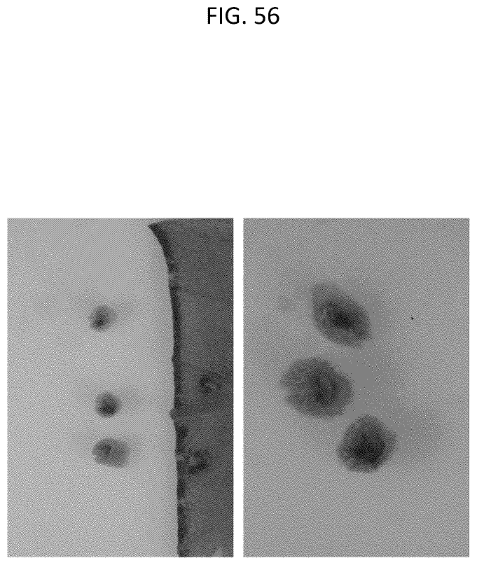

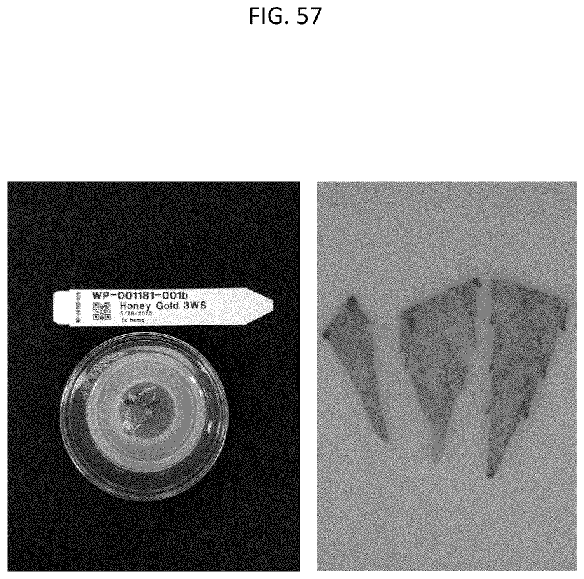

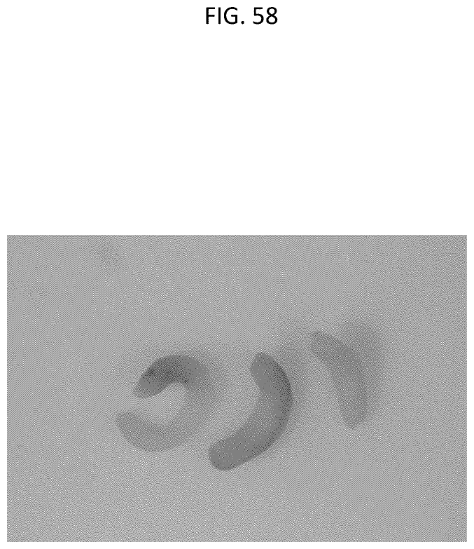

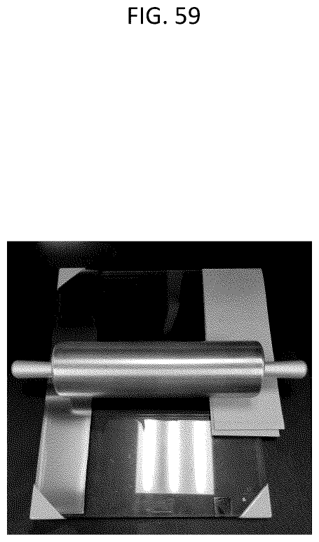

Previous examples described taking petiole sections of the Cannabis T0 event WP421-1 to examine GUS expression in vascular tissue, which appeared to be an early indicator of germline status in the Cannabis WP421-1 T0 event, and found GUS expression to be present in vascular tissue of WP001181-la as well (FIG. 56). The second piece of this explant rooted approximately 3 weeks after the first, and was sent to greenhouse after finding all leaves GUS+ (FIG. 57). The ability to split the event into two T0 plants in the meristem transformation system may offer some downstream advantages. It may be possible to feminize either the WP-001181-1a or WP-001181-1b to make subsequent flowers male (process described later). Crosses from -1a onto -1b would then yield homozygotes in the T1 progeny, which may offer timing advantages for transgenic Cannabis breeding programs (and advantages to attempting to make hermaphroditic plants for same purpose).

DNA complexed with gold “bead prep” was made by these steps:
Sonicate 50 mg/1 ml EtOH gold suspension for 3 min 45-55 kHz to resuspend gold.
Transfer 42 ul to a new tube. Pellet by centrifugation at 2500 rpm for 10 seconds, remove EtOH.
Add 500 uL sterile water, sonicate 3 min 45-55 kHz. Pellet by centrifugation at 2500 rpm for 10 seconds, remove water.
Add 25 ul sterile water (wash sides of tube with pipette tip). Sonicate 3 min 45-55 kHz.
Add 2.6 ug DNA
Add cold sterile water to bring volume up to 245 ul.
Add 250 ul cold 2.5 M CaCl2.
Add 50 ul 0.1 M spermidine
Mix solution by low speed vortexing. Incubate tube on ice for at least 45 min (I usually go 1.5 hrs). Invert tube every 5-10 minutes.
Once coating is done, pellet the DNA/gold at 1000 rpm (˜100×g) for 2 min. Remove supernatant.
Wash pellet with 1 ml 100% EtOH w/pipette tip. Pellet DNA/gold at 1000 rpm (˜100×g) for 2 min. Remove EtOH.
Add 36 ul 100% EtOH, completely resuspend gold with low-speed vortexing.
Store at −20 C, we generally use 5 ul of this prep per bombardment.
This DNA prep gives a DNA Loading Rate of 1.2 ng DNA/ug gold (360 ng DNA per blast, 290 ug gold per blast).

CMC targeting media includes 8% low viscosity carboxymethylcellulose, 2% medium viscosity carboxymethylcellulose, and 0.4% washed agar. CMC was made by adding washed agar to water, autoclaving, pouring into blender, adding carboxymethylcellulose, blending, pouring into bottle, then re-autoclaving, then poured into plates or stored.

EJW1 media is shown above in Table 10.

TABLE 19
BRM media

| Ingredients and Notes | Amount to add per liter (grams) |
|---|---|
| MS Salts no vitamins M524 | 2.15 |
| myo-inositol | 0.1 |
| sucrose | 30 |
| pH 5.8 with KOH | |
| Agar, Sigma A7921 | 8 |
| Autoclave | |
| Add after autoclaving | |
| Cysteine (100 mg/ml) - make | Use at 1.0 ml per Liter (100 mg/L) |
| stock fresh | |
| Cefotaxime (100 mg/ml) | Use at 2.0 ml per Liter (200 mg/L) |
| IAA (1 mg/ml) | Use at 0.1 ml per Liter (0.1 mg/L) |
| MS Vitamins (1000X) | Use at 1.0 ml per Liter |
| Selection | as needed |

It should also be possible to transform Cannabis meristem explants with free DNA methods where the explants do not require targeting (i.e. nanotechnology such as cell penetrating peptides, silica carbide fibers or others; as well as use of lipid and/or cationic lipid compounds).

Transient transformation of storable Cannabis meristem explants excised from seed using automation: We have demonstrated transient expression of GUS in meristem explants of the Fiber Hemp varety using meristem explants excised from seed using automation (described in comments to application sent in July 2019) with H1-H5 fractions pooled. These explants had been stored at −20 C for x weeks before rehydration in INO+60 mg/L Cleary's and inoculation with Ar18r12v/DICOTBINARY-19. Presence of GUS also indicates cells remained viable during this ˜8 month storage time. (FIG. 58).

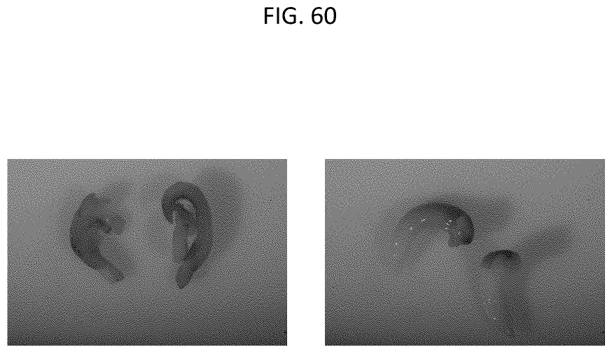

Cannabis seeds can also be crushed to extract mature embryos using a metal rolling pin on a glass plate, with an adjustable gap made using shims (FIG. 59). Cannabis seed of the Abacus variety was sanitized and imbibed overnight at 23 C. Seed was then either crushed under the rolling pin, with crushed material allowed to dry in a laminar flow hood for 72 hours; or seed was dried first for 72 hours in laminar flow hood and then crushed. Crushed material was stored at −20 C for 2 months, then rehydrated in INO media with 60 mg/L Cleary's fungicide. Explants were inoculated with Ar18r12v/DICOTBINARY-19 and co-cultured as previously described. We noticed transient GUS expression in meristem explants, again indicating cells were capable of surviving storage as well as maintaining some competency for Agrobacterium transformation (FIG. 60). This crushing technology and roller technology can be applied to wet or dry Cannabis seed, and can be used in conjunction with other treatments to compromise seed coat (ex. seed placed in paint shaker with a metal bead or beads).

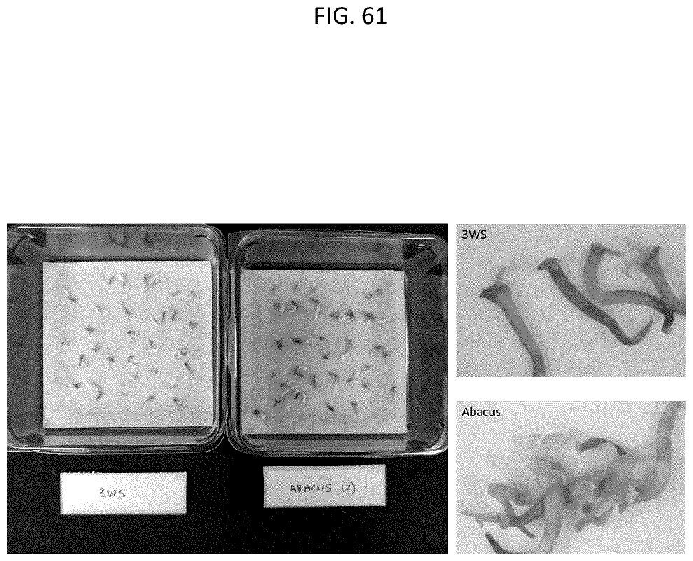

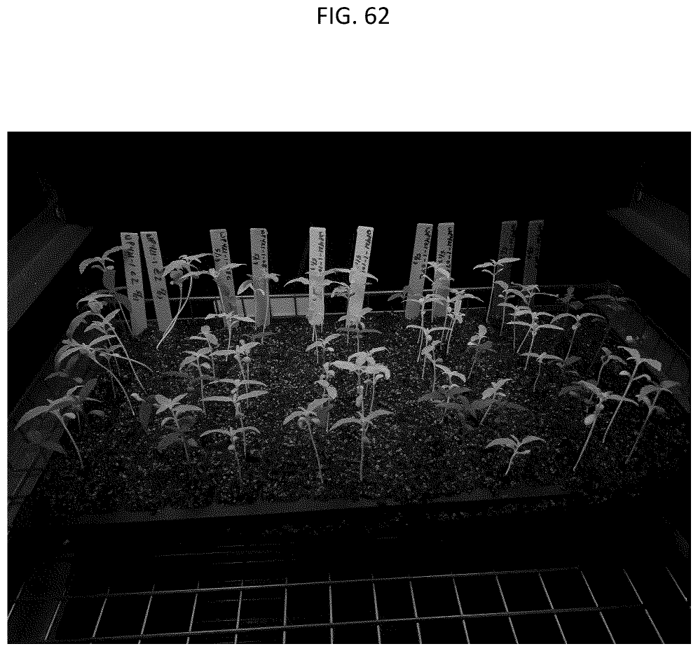

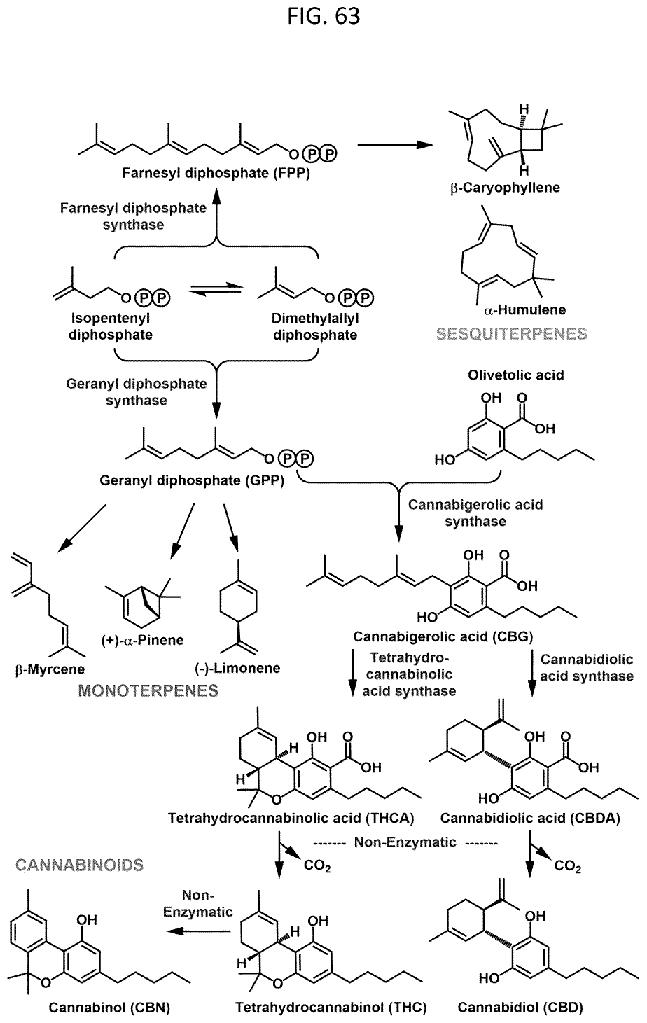

Transient transformation of elite Abacus Cannabis variety using Agrobacterium-mediated transformation of meristem explants: We have also demonstrated transient expression of GUS in meristem explants of the elite Abacus variety using meristem explants inoculated with Ar18r12v/DICOTBINARY-19 (derived from seed imbibed overnight at 23 C) (FIG. 61).

Transmission of transgenes into T2 generation of Cannabis 3WS variety (from initial T0 event WP421-1): The previously described GUS positive, RFP (tdTomato) positive, and aadA positive T1 plant WP421-1@2 and cuttings from this plant (WP421-1-08C; WP421-1-10C; WP421-1-07C; WP421-1-02C) were used to examine transmission of transgenes into the T2 generation. This T1 plant and its cuttings were pollinated with pollen from a separate GUS positive and RFP positive feminized WP421-1 T1 plant. Feminization stresses the female plant out enough that it is making male structures that have female genetic pollen. Seed is feminized from the subsequent crosses (all female). Three main substances can be used: Colloidal Silver Solution (˜120 ppm); Silver Thiosulfate/Silver Nitrate (STS); or GA3. These chemicals reduce ethylene production that ordinarily helps ripening and the production of female flowers. Temperature or other stresses can cause feminization as well. This T1 plant was feminized by the following procedure. 5 days before moving plants to short days (i.e., 12 hour light periods), all branches and leaves of the plants were sprayed to saturation with ether the STS or Colloidal Silver solution. The spraying was repeated every 5 days until major male flower formation has begun. Usually male flowers begin showing at about 16-20 days after initial spraying and spraying finished after 25-30 days. Resulting T2 seeds were harvested and germinated nad imaged for RFP expression. These crosses gave rise to the expected 3:1 segregation ratio (from both parents segregating 1:1), and the 3:1 ratio should include 1 homozygote, 2 hemizygotes and 1 null at T2.

TABLE 20
RFP segregation ratios in T2 Cannabis lines
derived from transformed meristem explants.

| Cannabis line | Ratio |
|---|---|
| WP421-1@2 | 10/15 positive |
| WP421-1-08C | 13/18 positive |
| WP421-1-10C | 16/18 positive |
| WP421-1-07C | 13/18 positive |
| WP421-1-02C | 12/16 positive |
| Total | 64/85 positives |

### Example 9

The reduction or elimination of THC will be accomplished by knocking out the THCA synthase gene using a CRISPR/Cas9 gene editing approach. Cannabis has two genes that contribute to THC production, namely THCA synthase (primary) and CBDA synthase (secondary). It is postulated that in high CBD varieties of Cannabis, the THCA synthase gene(s) are either inactive or highly suppressed. Since CBDA synthase has about an 84% amino acid similarity to THCA synthase, it is also possible that it also plays a role in producing the low amounts of THC seen in these varieties. The proposed CRISPR approach includes a gRNAs designed to target both the THCA synthase as well as the CBDA synthase resulting in plants with single gene knockouts as well as knockouts for both genes. Plants produced from this editing approach would be analyzed with our UPLC to determine the content of 13 different cannabinoids including THCA, CBDA, and CBG. In general, high CBD varieties of Cannabis have a CBDA:THCA ratio of 20:1 to 27:1 and these compounds are tightly related to each other (i.e. as CBDA goes up so does THCA). A potential outcome of this editing approach may also be a plant that is skewed with a higher ratio of CBDA:THCA, again reducing the risk to the grower of cultivating plants producing THC as well as producing more CBDA. Another possible outcome of knocking out both THCA synthase and CBD synthase would be the accumulation of the precursor compound CBG. A plant high in CBG is also a highly valued product due to the fact that the plant would normally make very little CBG and thus is a cannabinoid in higher demand.

A big risk to farmers trying to maximize their CBD amounts in their Cannabis plants, is pollination from male plants in the population or form adjacent fields. Pollination of female plants substantially reduces the amount of CBD made by the plants due to the reduction of trichome producing pistils and energy going toward making seed, which is low in CBD. Huang et al. successfully expressed the Solo Dancers and Barnase genes using a fusion gene, which resulted in fully sterile male/female flowers in Arabidopsis and tobacco without affecting growth or development. Since Cannabis can be easily cloned from a “mother” plant as demonstrated herein, a female sterile plant could be of high value to the grower in eliminating the risk of pollination to the crop.

Increasing trichome numbers: Cannabinoids are mainly made and secreted in the trichomes of the Cannabis plant, and more specifically the trichomes associated with female flowers (pistils) of the plant. Increasing the number of trichomes will increase the total cannabinoid production in the plant. Tian, et al. successfully overexpressed the BraLTP2, a lipid transfer protein from Brassica napus, resulting in a 10-fold increase of trichomes in B. napus. The transgenic lines also produce elevated levels of 43 different secondary metabolites, which could also prove interesting to the amount and type of cannabinoids made in a transgenic Cannabis plant with overexpression of this type of gene. LTPs belong to a large multigene family with many complex physiological functions. We have identified similar genes in the Cannabis genome and propose to overexpress both the B. napus BraLTP2 as well as some homologues from Cannabis. These genes could be driven by either a constitutive or flower/pistil specific promoter to drive these potential outcomes. Transgenic plants would be visually screened for an increase in trichomes as well as tested for cannabinoid content using our UPLC.

High CGBA plants: The CsPT1 ((geranylpyrophosphate: olivetolate geranyltransferase (GOT)) gene is involved in the production of CBGA, the precursor to THCA and CBDA. By overexpressing this gene, it may be possible to increase the amount of CBGA in the plant resulting in the increase of the downstream cannabinoids CBDA ad THCA. This approach could also be used in conjunction with knocking out the THCA synthase gene and ultimately making a very high CBDA plant with little or no THCA.

Herbicide resistant plants: Like many other plants, Cannabis has an EPSP Synthase gene. It is known that changes to 1-3 amino acids in this gene tend to confer glyphosate resistance to the plant. Since weed pressure in Cannabis is high due to the competition of weeds before its canopy is established, this would be a good target. Currently there are no federally registered pesticides for Cannabis. A gene editing is used to make the required amino acid substitutions, and produce a glyphosate resistant Cannabis plant.

### Example 10

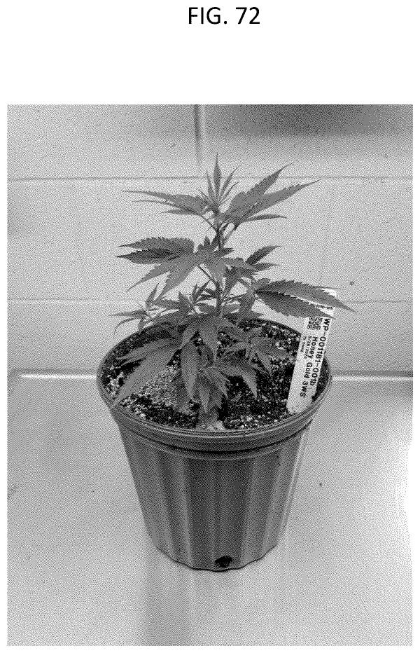

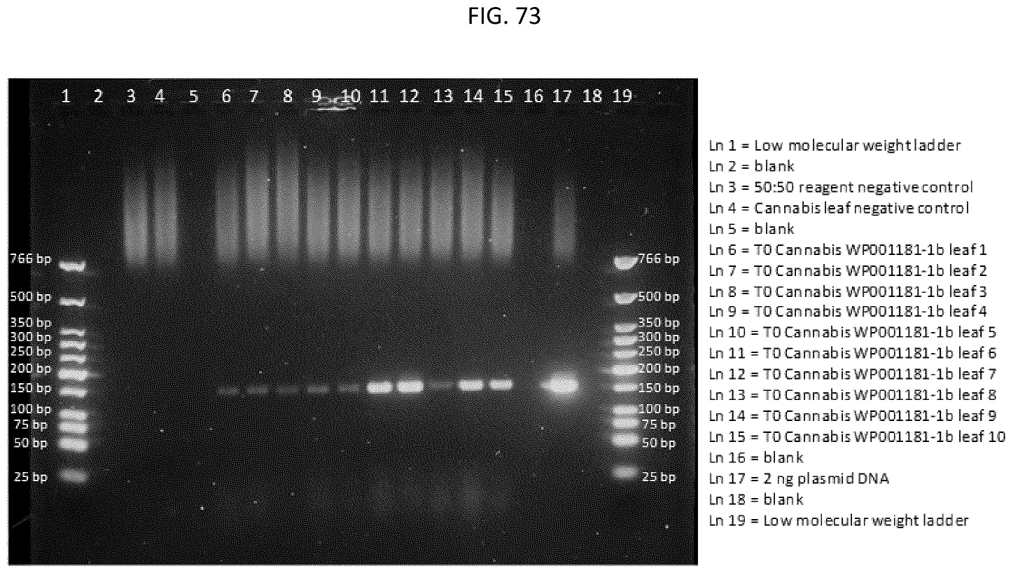

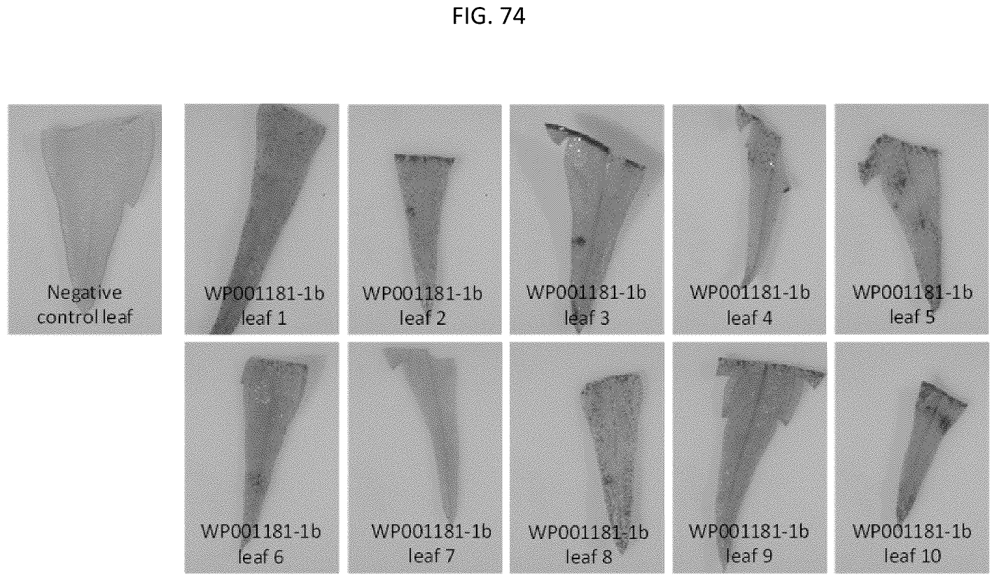

Transgenic T0 Cannabis plant generation using particle-mediated transformation of Cannabis meristem explants—We tested 10 leaves of the twin particle gun event described in Example 8, WP-001181-1b “Hernanda” for aadA1a by PCR and GUS by expression and all 10 leaves were positive. (FIGS. 72-74). As described herein (Example 2), it is also possible to transform plastids and/or proplastids of Cannabis meristem explants using plastid specific promoters driving aadA (and GOI) such as the large subunit promoter of RUBISCO; or a suitable prokaryotic promoter. This would give advantages of plastid transformation (maternal inheritance with null pollen, whole operon engineering, greater protein expression, etc.) in Cannabis and Cannabis breeding.

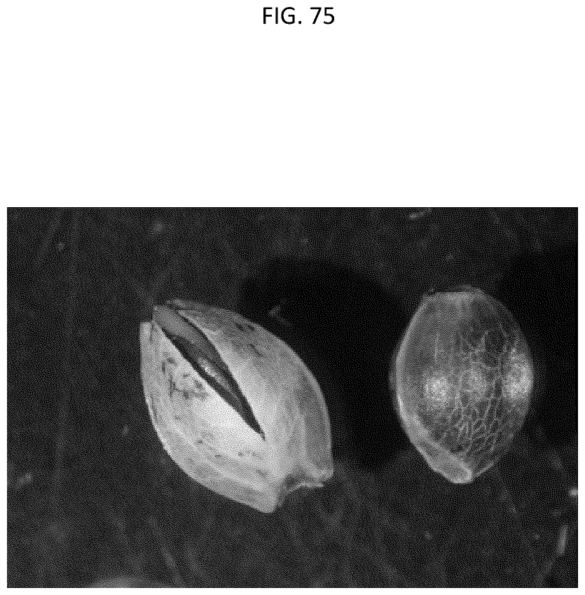

Alternate seed conditioning without imbibition-We were able to get radical emergence from Cannabis seed of the Badger variety using seed sanitization followed by incubation at 37° C. without imbibition. This enables new forms of both hand and machine excision and storage (as seed coat is split but at relatively low moisture compared with imbibed seed). (FIG. 75)

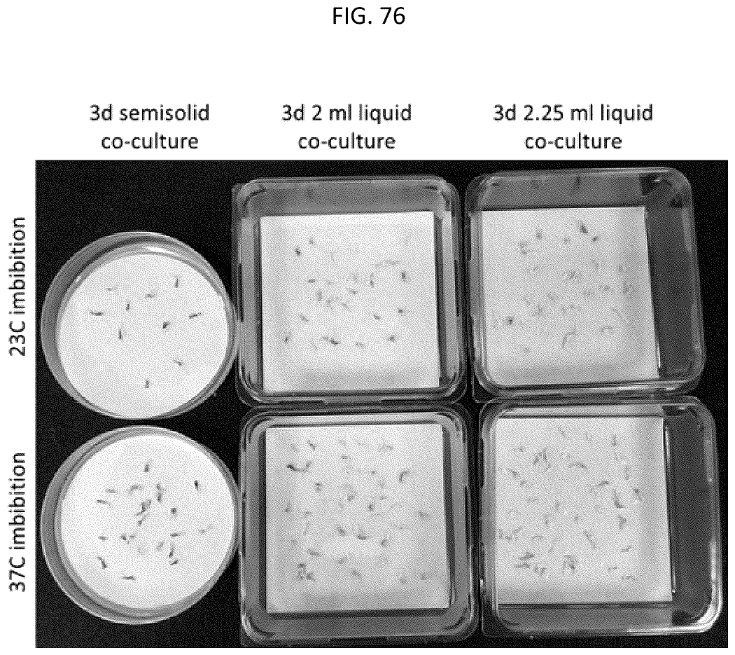

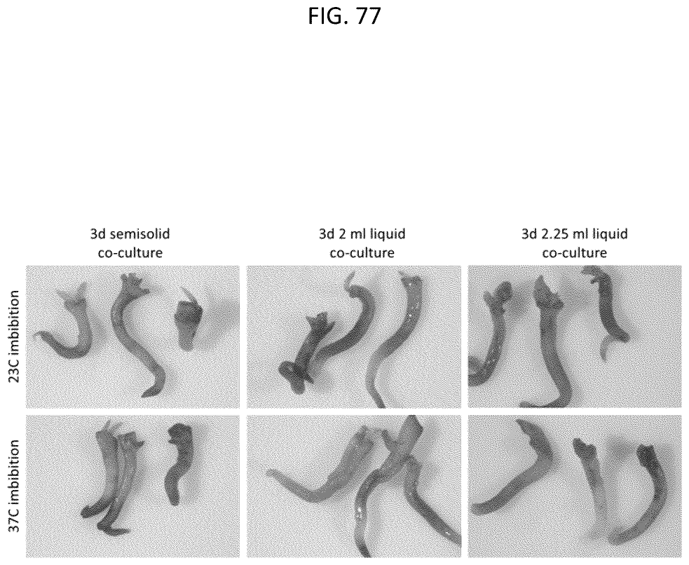

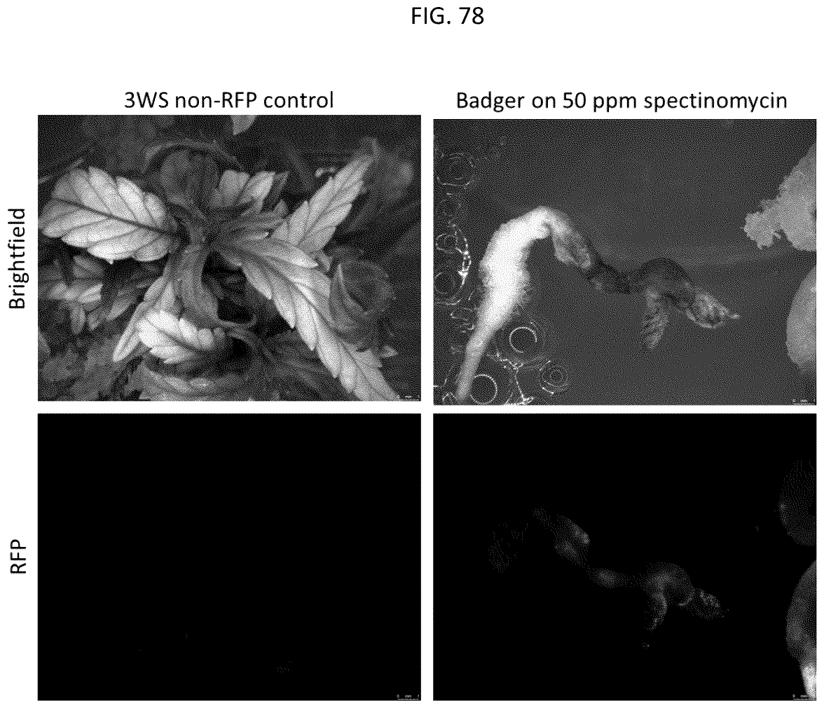

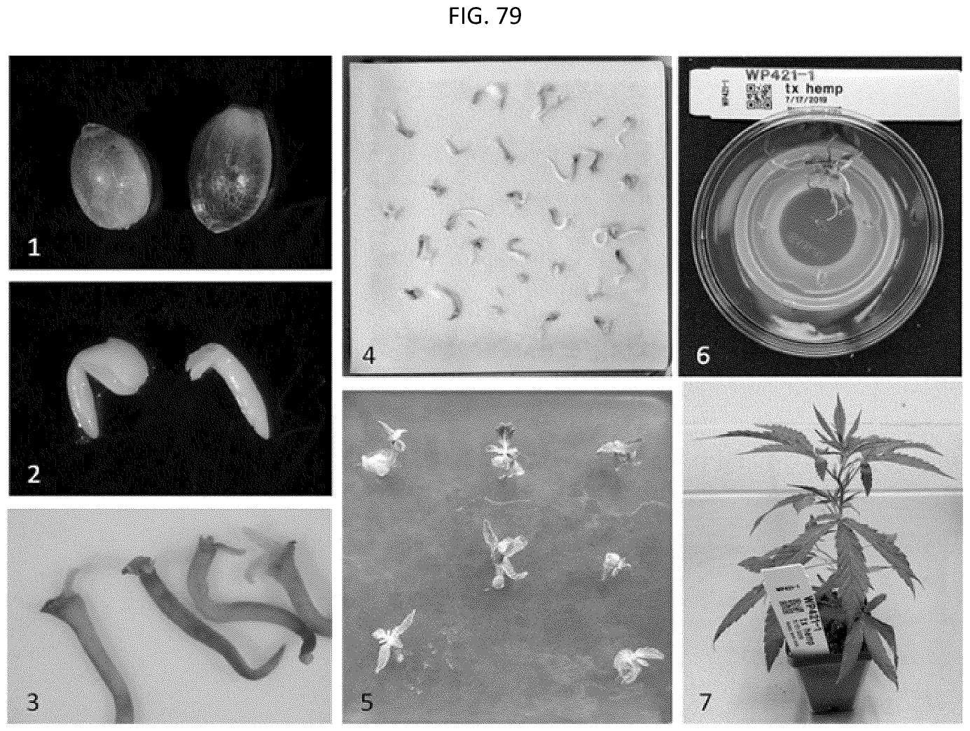

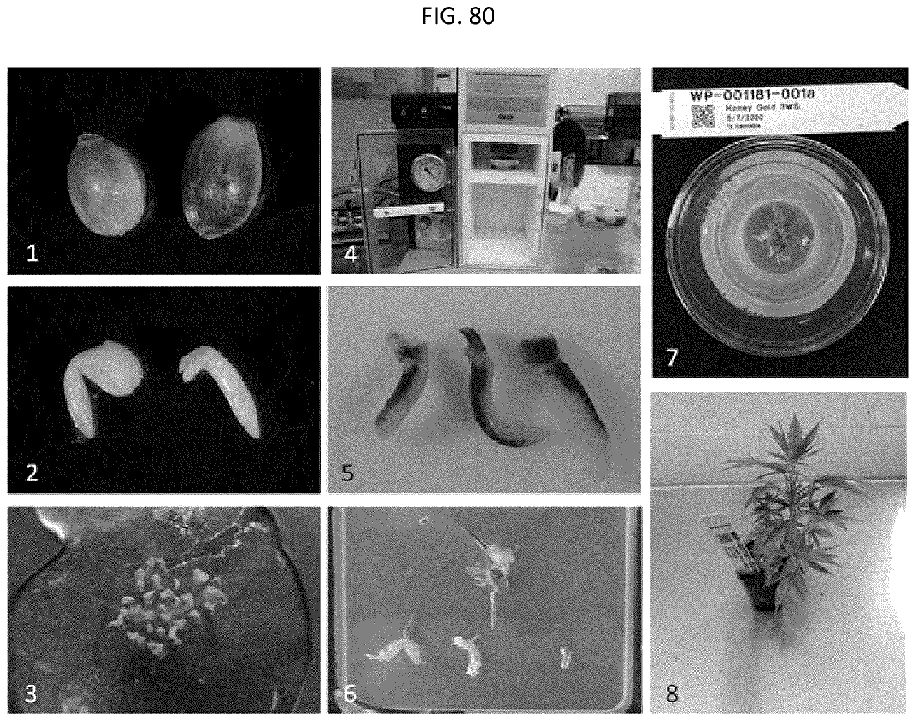

Transient transformation and Stable expression of RFP (tdTomato) in Badger variety using Agrobacterium-mediated transformation of meristem explants-We have also demonstrated transient expression of GUS in meristem explants of the elite Badger variety using meristem explants inoculated with Ar18r12v/DICOTBINARY-19 (derived from seed imbibed overnight at 23° C. or 37° C.) under a variety of co-culture conditions (FIGS. 76-78).

1-34. (canceled)

35. A genetically engineered Cannabis plant comprising a genetic modification in the nucleic acid encoding at least one protein selected from the group consisting of tetrahydrocannabinolic acid synthase (THCA synthase) and cannabidiolic acid synthase (CBDA), wherein the expression or enzyme activity of the protein in the plant is modified relative to a control Cannabis plant with wild-type expression or enzyme activity of at least one of THCA synthase and CBDA synthase.

36. The plant of claim 35, comprising genetic modifications in the nucleic acid encoding the THCA synthase and CBDA synthase, wherein the expression or enzyme activity of THCA synthase and CBDA synthase in the plant are modified relative to a control Cannabis plant with wild-type expression or enzyme activity of THCA synthase and CBDA synthase.

37. The plant of claim 35, further comprising a genetic modification in the nucleic acid encoding at least one additional protein selected from the group consisting of O-methyltransferase (CsOMT21), lipid transfer protein 2 (LTP2), prenyltransferase 3 (CsPT3), the EPSP synthase, and prenyltransferase 1 (CsPT1), wherein the expression or enzyme activity of the additional protein in the plant is modified relative to a control Cannabis plant with wild-type expression or enzyme activity of at least one of CsOMT21, LTP2, CsPT3, EPSP, and CsPT1.

38. The plant of claim 35, wherein the plant was generated from a genetically engineered explant.

39. The plant of claim 38, wherein the explant is selected from the group consisting of a leaf explant, a node explant, an internode explant, a petiole explant, a hypocotyl explant, a bud explant, a meristem explant, and a pollen explant.

40. The plant of claim 38, wherein the explant is genetically engineered using CRISPR/Cas mediated gene editing.

41. The plant of claim 40, wherein one or more guide RNAs target the at least one protein to reduce or eliminate expression or enzyme activity of the at least one protein.

42. The plant of claim 40, wherein one or more guide RNAs target the THCA synthase gene, or a region adjacent there to, and proximal to a PAM site.

43. The plant of claim 40, wherein one or more guide RNAs target the CBDA synthase gene, or a region adjacent there to, and proximal to a PAM site.

44. The plant of claim 40, wherein the guide RNA targeting the THCA synthase gene is selected from the group consisting of SEQ ID NO:7, SEQ ID NO:8, SEQ ID NO:9, SEQ ID NO:10, and combinations thereof; and/or wherein the guide RNA targeting the CBDA synthase gene is selected from the group consisting of SEQ ID NO:18, SEQ ID NO:19, SEQ ID NO:20, SEQ ID NO:21, and combinations thereof.

45. The plant of claim 40, wherein the guide RNA targeting both the THCA synthase gene and the CBDA synthase gene is selected from the group consisting of SEQ ID NO:22, SEQ ID NO:23, SEQ ID NO:24, SEQ ID NO:25, SEQ ID NO:26, SEQ ID NO:27, and combinations thereof.

46. The plant of claim 40, wherein the THCA synthase or the CBDA synthase expression are reduced in the gene edited plant as compared to a control plant.

47. The plant of claim 35, wherein plant comprises a heterologous nucleic acid and the expression or enzyme activity of at least one of THCA synthase and CBDA synthase is modified in the plant relative to a control Cannabis plant with wild-type expression or enzyme activity of at least one of THCA synthase and CBDA synthase.

48. The explant of claim 46, wherein the heterologous nucleic acid encodes the at least one protein and increases the expression or activity of at least one of THCA synthase gene and CBDA synthase gene.

49. The plant of claim 35, wherein the plant has increased cannabigerolic acid (CBGA) as compared to a control Cannabis plant with wild-type expression or enzyme activity of at least one of THCA synthase and CBDA synthase.

50. The plant of claim 35, wherein the wild-type THCA synthase protein comprises SEQ ID NO: 6 or sequences with at least 95% sequence identity to SEQ ID NO: 6.

51. The plant of claim 35, wherein the wild-type CBDA synthase protein is selected from the group consisting of SEQ ID NOs: 12, 14, 16 and sequences with at least 95% sequence identity to SEQ ID NO:12, 14 and 16.

52. The plant of claim 37, wherein the additional protein is selected from the group consisting of SEQ ID NOs: 28, 30, 32, 34, 36, 37 and sequences having at least 95% identity to SEQ ID NO: 28, 30, 32, 34, 36, and 37.
